(19) United States
(12) Patent Application Publication
(10) Pub. No.: US 2021/0222204 A1
(43) Pub. Date: Jul. 22, 2021

(54) CATALYTICALLY INACTIVE TRUNCATED GUIDE RNA COMPOSITIONS AND RELATED METHODS FOR SUPPRESSION OF CRISPR/CAS OFF-TARGET EDITING

(71) Applicant: University of Washington, Seattle, WA (US)

(72) Inventors: John C. Rose, Seattle, WA (US); Dustin James Maly, Seattle, WA (US); Douglas Fowler, Seattle, WA (US); Nicholas Popp, Seattle, WA (US)

(73) Assignee: University of Washington, Seattle, WA (US)

(21) Appl. No.: 17/144,944

(22) Filed: Jan. 8, 2021

Related U.S. Application Data
(60) Provisional application No. 62/959,710, filed on Jan. 10, 2020.

(51) Int. Cl. C12N 15/90; C12N 9/22; C12N 15/11

(52) U.S. Cl. CPC C12N 15/907; C12N 9/22; C12N 2800/80; C12N 2310/20; C12N 15/11

(57) ABSTRACT

The disclosure provides compositions and methods for suppressing off-target editing guide RNA-nuclease complexes. The disclosed strategies incorporate use of catalytically inactive truncated guide RNA/nuclease complexes to shield off-target editing. In some embodiments, the disclosure provides a method of inhibiting off-target cleavage of DNA by a first guide RNA-endonuclease complex by contacting the DNA with a second guide RNA-endonuclease complex that comprises a second guide RNA corresponding to the off-target site but with a recognition sequence of 16 or fewer nucleotides. In another aspect, the disclosure provides a method for preventing cleavage of DNA after editing and subsequent homology-directed repair (HDR) by contacting the repaired DNA with a guide RNA-endonuclease complex that comprises a guide RNA with a guide RNA corresponding to the repaired sequence but with a recognition sequence of 16 or fewer nucleotides. Additional methods, compositions, and kits are also provided.

### CROSS-REFERENCE TO RELATED APPLICATION

This application claims the priority benefit of U.S. Provisional Application No. 62/959,710, filed Jan. 10, 2020, which is incorporated herein by reference for all purposes.

### STATEMENT OF GOVERNMENT LICENSE RIGHTS

This invention was made with Government support under Grant Nos. F30 CA189793 and RO1 GM109110, awarded by the National Institutes of Health and Grant No. 0954242, awarded by the National Science Foundation. The Government has certain rights in the invention.

### STATEMENT REGARDING SEQUENCE LISTING

The sequence listing associated with this application is provided in text format in lieu of a paper copy and is hereby incorporated by reference into the specification. The name of the text file containing the sequence listing is UWOTL173209_Sequence_final_20210108.txt. The text file is 50 KB; was created on Jan. 8, 2021; and is being submitted via EFS-Web with the filing of the specification.

### BACKGROUND

The Cas9 nucleases such as S. pyogenes Cas9 (SpCas9) is targeted to specific sites in the genome by a single guide RNA (sgRNA) containing a 20-nucleotide target recognition sequence. The target site must also contain an NGG protospacer adjacent motif (PAM). This multipartite target recognition system is imperfect, and most sgRNAs direct significant cleavage and subsequent unwanted editing at off-target sites whose sequence is similar to the target site. Numerous approaches to reduce off-target editing have been devised yet are hampered by various limitations. For example, SpCas9 variants with improved specificity have been engineered. While useful, these high-specificity variants often decrease on-target editing, and in most cases, do not eliminate all unwanted editing. All high-specificity Cas9 variants appear to balance on- vs off-target activity via the same mechanism and, as a consequence, often fail to suppress editing at the same obstinate off-target sites.

Accordingly, despite the advances in the art of directed gene editing, a need remains for new methods for off-target suppression, particularly methods that preserve on-target editing, and which can be combined with high-specificity nucleases variants, while requiring minimal expenditure of time, effort, and resources. The present disclosure addresses these and related needs.

### SUMMARY

This summary is provided to introduce a selection of concepts in a simplified form that are further described below in the Detailed Description. This summary is not intended to identify key features of the claimed subject matter, nor is it intended to be used as an aid in determining the scope of the claimed subject matter.

In one aspect, the disclosure provides a method of inhibiting off-target cleavage of a DNA molecule by a first guide RNA-endonuclease complex, wherein the first guide RNA-endonuclease complex comprises a first guide RNA comprising a nucleotide target recognition sequence complementary to a first target sequence. The method comprises contacting the DNA molecule with a second guide RNA-endonuclease complex, wherein second guide RNA-endonuclease complex comprises a second guide RNA comprising a nucleotide target recognition sequence with 16 or fewer nucleotides and is complementary to a second target sequence in the DNA molecule. The second target sequence is different from the first target sequence but the second target sequence is capable of cleavage at a measurable rate by the first guide RNA-endonuclease complex.

In some embodiments, the method further comprises contacting the DNA molecule with the first guide RNA-endonuclease complex. In some embodiments, the second guide RNA-endonuclease complex is contacted to the DNA molecule prior to or simultaneously with the first guide RNA-endonuclease complex. In some embodiments, the first guide RNA-endonuclease complex and the second guide RNA-endonuclease complex are contacted to the DNA molecule at a ratio of about 20:1 to about 1:20. In some embodiments, the second target sequence differs from the first target sequence by 0-10 nucleotide mismatches.

In some embodiments, the first guide RNA-endonuclease complex comprises a first endonuclease and the second guide RNA-endonuclease complex comprises a second endonuclease, wherein the first endonuclease and the second endonuclease are clustered regularly interspersed short palindromic repeats (CRISPR)/CRISPR-associated (Cas) system proteins.

In some embodiments, the first endonuclease and the second endonuclease are independently selected from Cas12a, Cas9, or high-fidelity variants of Cas9 such as eSpCas9, SpCas9-HF1, HypaCas9, as well as xCas9, SpCas9-NG, and the like. In some embodiments, the first endonuclease is the same type of endonuclease as the second endonuclease. In some embodiments, the first endonuclease or the second endonuclease is derived from Streptococcus, e.g., Streptococcus pyogenes, Staphylococcus, e.g., Staphylococcus aureus, Neisseria, e.g., Neisseria meningitidis, Acidaminococcus species, or Lachnospiraceae species.

In some embodiments, contacting the DNA molecule with the second guide RNA-endonuclease complex reduces cleavage of the second target sequence by the first guide RNA-endonuclease complex by at least 10% compared to similar reaction conditions but wherein no second guide RNA-endonuclease complex is present. In some embodiments, the nucleotide target recognition sequence of the second guide RNA-endonuclease complex comprises between 10 and 16 nucleotides inclusive that are complementary to the second target sequence.

In some embodiments, the method is multiplexed with one or more additional guide RNA-endonuclease complexes, wherein each of the one or more additional complexes comprises a different nucleotide target recognition sequence with 16 or fewer nucleotides and is complementary to one or more additional target sites in the DNA molecule or a plurality of DNA molecules in a same reaction environment, wherein the one or more additional target sequences are different from each other and from the first target sequence but the additional target sequences are capable of cleavage at measurable rates by the first guide RNA-endonuclease complex.

In some embodiments, the DNA molecule is in a cell, and wherein contacting the DNA molecule with the second guide RNA-endonuclease complex comprises contacting the cell with one or more exogenous nucleic acid molecules comprising a first sequence encoding the second guide RNA and a second sequence encoding the second endonuclease, wherein upon expression of the first sequence and the second sequence the second guide RNA and the second endonuclease form the second guide RNA-endonuclease complex in the cell. In some embodiments, the DNA molecule is in a cell, and wherein contacting the DNA molecule with the second guide RNA-endonuclease complex comprises contacting the cell with a pre-assembled second guide RNA-endonuclease complex.

In some embodiments, the DNA molecule is in a cell, and wherein contacting the DNA molecule with the first guide RNA-endonuclease complex comprises contacting the cell with one or more exogenous nucleic acid molecules comprising a first sequence encoding the first guide RNA and a second sequence encoding a first endonuclease, wherein upon expression of the first sequence and the second sequence the first guide RNA and the first endonuclease form the first guide RNA-endonuclease complex in the cell. In some embodiments, the DNA molecule is in a cell, and wherein contacting the DNA molecule with the first guide RNA-endonuclease complex comprises contacting the cell with a pre-assembled first guide RNA-endonuclease complex.

In another aspect, the disclosure provides a method of inhibiting cleavage of a DNA molecule at a target site that has been previously modified from containing a first sequence to containing a second sequence by targeted cleavage by a first guide RNA-endonuclease complex and subsequent homology-directed repair (HDR), wherein the first guide RNA-endonuclease complex comprises a first guide RNA comprising a nucleotide target recognition sequence complementary to the first sequence. The method comprises contacting the DNA molecule with a second guide RNA-endonuclease complex, wherein the guide RNA of the second guide RNA-endonuclease complex comprises a second guide RNA comprising a nucleotide target recognition sequence with 16 or fewer nucleotides and is complementary to at least a portion of the second sequence in the DNA molecule, wherein the second sequence is different from the first sequence but the second sequence is capable of cleavage at a measurable rate by the first guide RNA-endonuclease complex.

In some embodiments, the method further comprises inducing targeted cleavage of the DNA molecule containing the first sequence by contacting the DNA molecule with the first guide RNA-endonuclease complex, thereby producing a cleaved DNA molecule. In some embodiments, the method further comprises contacting the cleaved DNA molecule with a repair polynucleotide that is substantially homologous to the target site but comprises the second sequence. In some embodiments, the second sequence differs from the first sequence by 0-10 nucleotide mismatches.

In some embodiments, the first guide RNA-endonuclease complex comprises a first endonuclease and the second guide RNA-endonuclease complex comprises a second endonuclease, wherein the first endonuclease and the second endonuclease are clustered regularly interspersed short palindromic repeats (CRISPR)/CRISPR-associated (Cas) system proteins. In some embodiments, the first endonuclease and second endonuclease are independently selected from Cas12a, Cas9, or high-fidelity variants of Cas9 such as eSpCas9, SpCas9-HF1, HypaCas9, as well as xCas9, SpCas9-NG, and the like. In some embodiments, the first endonuclease is the same type of endonuclease as the second endonuclease. In some embodiments, the first endonuclease or the second endonuclease is derived from Streptococcus, e.g., Streptococcus pyogenes, Staphylococcus, e.g., Staphylococcus aureus, Neisseria, e.g., Neisseria meningitidis, Acidaminococcus species, or Lachnospiraceae species.

In some embodiments, contacting the DNA molecule containing the second sequence with the second guide RNA-endonuclease complex reduces cleavage of the second sequence by the first guide RNA-endonuclease complex by at least 10% compared to similar reaction conditions but wherein no second guide RNA-endonuclease complex is present. In some embodiments, the nucleotide target recognition sequence of the second guide RNA-endonuclease complex comprises between 10 and 16 nucleotides inclusive that are complementary to the second sequence.

In some embodiments, the DNA molecule is in a cell, and wherein contacting the DNA molecule with the second guide RNA-endonuclease complex comprises contacting the cell with one or more exogenous DNA molecules comprising a first sequence encoding the second guide RNA and a second sequence encoding the second endonuclease, wherein upon expression of the first sequence and the second sequence the second guide RNA and the second endonuclease form the second guide RNA-endonuclease complex in the cell. In some embodiments, the DNA molecule is in a cell, and wherein contacting the DNA molecule with the second guide RNA-endonuclease complex comprises contacting the cell with a pre-assembled second guide RNA-endonuclease complex.

In some embodiments, the DNA molecule is in a cell, and wherein contacting the DNA molecule with the first guide RNA-endonuclease complex comprises contacting the cell with one or more exogenous DNA molecules comprising a first sequence encoding the first guide RNA and a second sequence encoding a first endonuclease, wherein upon expression of the first sequence and the second sequence the first guide RNA and the first endonuclease form the first guide RNA-endonuclease complex in the cell. In some embodiments, the DNA molecule is in a cell, and wherein contacting the DNA molecule with the first guide RNA-endonuclease complex comprises contacting the cell with a pre-assembled first guide RNA-endonuclease complex.

In another aspect, the disclosure provides a composition comprising a first guide RNA-endonuclease complex and a second guide RNA-endonuclease complex. The guide RNA of the first guide RNA-endonuclease complex comprises a nucleotide target recognition sequence complementary to a first target sequence in a DNA molecule. The guide RNA of the second guide RNA-endonuclease complex comprises a nucleotide target recognition sequence with 16 or fewer nucleotides and is complementary to a second target site in the DNA molecule or a distinct DNA molecule. The second target sequence is different from the first target sequence but the second target sequence is capable of cleavage at a measurable rate by the first guide RNA-endonuclease complex.

In another aspect, the disclosure provides a plasmid comprising one or more nucleic acid domains encoding a first guide RNA, a second guide RNA, and an endonuclease, each operatively linked to a promoter sequence. The first guide RNA comprises a nucleotide target recognition sequence complementary to a first target sequence and the second guide RNA comprises a nucleotide target recognition sequence with 16 or fewer nucleotides and is complementary to a second target site. The second target sequence is different from the first target sequence but the second target sequence is capable of cleavage at a measurable rate by a complex of the first guide RNA and the endonuclease.

In another aspect, the disclosure provides a kit comprising: a first guide RNA-endonuclease complex and a second guide RNA-endonuclease complex, wherein the first guide RNA-endonuclease complex comprises a first guide RNA comprising a nucleotide target recognition sequence complementary to a first target sequence in a DNA molecule, wherein the second guide RNA-endonuclease complex comprises a second guide RNA comprising a nucleotide target recognition sequence with 16 or fewer nucleotides and is complementary to a second target site in the DNA molecule, and wherein the second target sequence is different from the first target sequence but the second target sequence is capable of cleavage at a measurable rate by the first guide RNA-endonuclease complex. The kit can comprise written indicia for inhibiting off-target cleavage of a DNA molecule by the first guide RNA-endonuclease complex and/or for implementing HDR without inclusion additional mutations to block recutting by the first guide RNA-endonuclease.

In another aspect, the disclosure provides a kit comprising one of the following:

the plasmid of described herein;

a first vector comprising nucleic acid domains encoding a first guide RNA and an endonuclease each operatively linked to a promoter sequence, and a second vector comprising nucleic acid domains encoding a second guide RNA and an endonuclease each operatively linked to a promoter sequence, wherein the first guide RNA comprises a nucleotide target recognition sequence complementary to a first target sequence and the second guide RNA comprises a nucleotide target recognition sequence with 16 or fewer nucleotides and is complementary to a second target site, and wherein the second target sequence is different from the first target sequence but the second target sequence is capable of cleavage at a measurable rate by a complex of the first guide RNA and the endonuclease; and a first vector comprising a nucleic acid domain encoding a first guide RNA operatively linked to a promoter sequence, a second vector comprising a nucleic acid domain encoding a second guide RNA operatively linked to a promoter sequence, and a third vector comprising a nucleic acid domain encoding endonuclease operatively linked to a promoter, wherein the first guide RNA comprises a nucleotide target recognition sequence complementary to a first target sequence and the second guide RNA comprises a nucleotide target recognition sequence with 16 or fewer nucleotides and is complementary to a second target site, and wherein the second target sequence is different from the first target sequence but the second target sequence is capable of cleavage at a measurable rate by a complex of the first guide RNA and the endonuclease.

The kit can further comprise written indicia for reducing or preventing off-target cleavage of a DNA molecule by the first guide RNA-endonuclease complex and/or for implementing HDR without inclusion additional mutations to block recutting by the first guide RNA-endonuclease.

### DESCRIPTION OF THE DRAWINGS

The foregoing aspects and many of the attendant advantages of this invention will become more readily appreciated as the same become better understood by reference to the following detailed description, when taken in conjunction with the accompanying drawings, wherein:

FIGS. 1A-1E: dRNA Mediated Off-Target Suppression (dOTS) effectively reduces off-target editing. (1A) Schematic representation of dOTS. A dRNA with perfect complementarity for an off-target site directs Cas9 binding but not cleavage, protecting the site. (1B) Indel frequencies and specificity ratios (on-target/off-target indel frequency ratios) at the FANCF sgRNA2 on-target site and OT1 24 hours after transfection of HEK-293T cells with Cas9, sgRNA, and FANCF sgRNA2 OT1 dRNA1 or a non-targeting control dRNA (dNT) that does not target genomic DNA. For conditions without dRNA, an equivalent amount of pMAX-GFP was substituted. Means of n=3 biological replicates depicted by solid lines. (1C) Normalized specificity ratios, computed as the specificity ratio of the best dRNA condition (TABLE 1) divided by the specificity ratio of the sgRNA only condition for 19 guide/off-target pairs tested in HEK-293T cells. Points depict the mean of n=3 biological replicates, error bars show the standard error of the mean. OT=off-target. (1D) Indel frequencies and specificity ratios at the FANCF sgRNA2 on-target site and OT1 24 hours after transfection in U2OS cells and (1E) Elf1 embryonic stem cells. Control samples to the right of the x-axis break were performed separately. iCas9 denotes stable integration of Cas9 under the control of a doxycycline-inducible promoter. Means of n=3 cell culture replicates depicted by solid lines.

FIGS. 2A and 2B: dRNAs affect off-target, but not on-target, editing kinetics and can be titrated to improve specificity. (2A) Editing of FANCF sgRNA2 on-target and OT1 sites using chemically inducible Cas9 (ciCas9) from 0 to 16 hours after activation with A115. Non-targeting dRNA is a 14-base control dRNA targeting a non-endogenous site. NT=non-transfected control. Points depict the mean of n=3 biological replicates. Error bars show the standard error of the mean. (2B) Indel frequencies and specificity ratios at VEGFA sgRNA3 on-target and OT2 sites in cells transfected with plasmids encoding Cas9 and varying ratios of VEGFA sgRNA3 and dRNA2. dRNA untreated cells were transfected with Cas9 and a 1:1 VEGFA sgRNA3:GFP plasmid ratio. Error bars depict s.e.m. (n=3 cell culture replicates). OT=off-target.

FIGS. 3A and 3B: dRNAs can be combined with other approaches for improving Cas9 specificity. Indel frequencies and specificity ratios 24 hours after transfection with (3A) plasmids encoding WT Cas9, a dRNA targeting VEGFA sgRNA3 OT2 (dRNA2) and a truncated guide VEGFA tru-sgRNA3 or (3B) High-specificity variants of Cas9 and a dRNA targeting FANCF sgRNA2 OT1 (dRNA1). WT=wildtype Cas9, E=eSpCas9, HF1=SpCas9-HF1, Hypa=HypaCas9. Means of n=3 cell culture replicates depicted by solid lines. OT=off-target.

FIGS. 4A and 4B: dRNAs can be multiplexed to suppress several off-targets simultaneously. Indel frequencies and specificity ratios 24 hours after transfection of plasmids encoding either (4A) wild type (WT) or (4B) eSpCas9 (E), VEGFA sgRNA2, and dRNAs targeting one of three VEGFA sgRNA2 off-targets (OT1 dRNA1, OT2 dRNA8, OT17 dRNA8). Means of n=3 cell culture replicates depicted by solid lines. OT=off-target.

FIGS. 5A-5C: dRNA On-target Recutting Suppression (dReCS) facilitates scarless HDR. (5A) Schematic depicting dReCS and alignment of BFP, GFP, sgRNA, and dRNA sequences. dRNA exhibiting perfect complementarity for the repaired site directs Cas9 binding but not cleavage, protecting the repaired site. Single base change to generate GFP from BFP is displayed in green with affected codon indicated by grey box. PAM sequences are underlined. Black arrow indicates best dRNA, as determined by maximal improvement in HDR yield. (5A) Indels and homology-directed repair (HDR) as assessed by flow cytometry, where indels lead to a loss of BFP signal, and HDR leads to a loss of BFP and gain of GFP signal. (5C) HDR as a percentage of total Cas9 edits observed. Means of n=3 cell culture replicates depicted by solid lines. dRNA=BFP sgRNA1 dRNA3 (see FIGS. 10A-10C, Supplementary Data Set).

FIGS. 6A-6C: FANCF dRNA1 does not promote Cas9-mediated editing. (6A) Sequence alignment of FANCF sgRNA2, its on-target site, the most prominent off-target, off-target site 1 (OT1), and multiple dRNAs complementary to OT1. Black arrows indicate best dRNA, as determined by maximal off-target editing suppression with minimal on-target editing suppression. (6B) Indel frequencies and specificity ratios (on-target/off-target indel frequency ratios) at the FANCF sgRNA2 on-target site and OT1 24 hours after transfection with Cas9, sgRNA, and various dRNAs. For conditions without dRNA, an equivalent amount of pMAX-GFP was substituted. (6C) Indel frequencies at the FANCF sgRNA2 on-target and OT1 sites 24 hours after transfection with Cas9 and dRNA1 but no sgRNA. The predicted cut sites of dRNA1 are the same as FANCF sgRNA2. Indel frequencies for untransfected cells are shown as a control. Numbers denote dRNA identity, see Supplementary Data Set 1. Solid lines denote the mean of n=3 biological replicates.

FIGS. 7A-7C: dRNA-mediated off-target editing suppression is durable. On-target and off-target indel frequencies and specificity ratios 72 hours after transfection with Cas9, sgRNA, and off-target specific dRNAs in HEK293T cells (7A) CCR5-R30 OT (CCR2). (7B) FANCF sgRNA2 OT1. (7C) HBB-R03 OT (HBD). Indel frequencies for untransfected cells are shown as a control. Numbers denote dRNA identity, see Supplementary Data Set. Solid lines denote the mean of n=3 biological replicates, except CCR5-R30 and VEGFA sgRNA3 without RNA where n=2.

FIG. 8: dOTS can suppress refractory off-target editing of high-specificity Cas9 variants. On-target and off-target indel frequencies and specificity ratios 24 hours after transfection of plasmids encoding VEGFA sgRNA3, dRNA and either wildtype Cas9 (WT), eSpCas9 (E), or SpCas9-HF1 (HF1). Indel frequencies for untransfected cells are shown as a control. Numbers denote dRNA identity, see Supplementary Data Set. Solid lines denote the mean of n=3 biological replicates. OT=off-target.

FIGS. 9A and 9B: multiple dRNAs can be combined to reduce unwanted editing at multiple refractory off-target sites of high-specificity Cas9 variants. Target and off-target indel frequencies and specificity ratios 24 hours after transfection with plasmids encoding VEGFA sgRNA2, a combination of three dRNAs and either (9A) SpCas9-HF1 (HF1) or (9B) HypaCas9 (Hypa). Despite being reported previously, indels were not observed at OT2, so specificity ratios were not plotted. Indel frequencies for untransfected cells are shown as a control. Solid lines denote the mean of n=3 biological replicates. OT=off-target.

FIGS. 10A-10C: screening guides, donors, and dRNAs for scarless HDR in a fluorescent reporter system. (10A) Screening of three dRNAs for indels or HDR events, percent HDR of total Cas9 editing observed, and fold change in HDR observed. (10B) Screening of various ratios of dRNA3 to sgRNA for NHEJ or HDR events, percent HDR of total Cas9 editing observed, and fold change in HDR observed. (10C) Comparison of symmetric donor and asymmetric donor for NHEJ or HDR events, percent HDR of total Cas9 editing observed, and fold change in HDR observed. HDR donors do not contain blocking mutations. Indel frequencies for untransfected cells are shown as a control. Numbers denote sgRNA and dRNA identities, see Supplementary Data Set. Solid lines denote the mean of n=3 biological replicates.

### DETAILED DESCRIPTION

CRISPR/Cas9 nucleases are powerful genome engineering tools, but unwanted cleavage at off-target and previously edited sites remains a major concern. Numerous strategies to reduce unwanted cleavage have been devised, but all are imperfect. The present disclosure describes the development of an orthogonal and general approach for suppressing off-targets that can be readily combined with existing methods, including high-specificity variants. As described in more detail below, off-target sites can be shielded from the active Cas9•single guide RNA (sgRNA) complex through the co-administration of “dead-RNAs” (dRNAs), which are truncated guide RNAs that direct Cas9 binding but not cleavage. It is demonstrated herein that dRNAs can effectively suppress a wide-range of off-targets with minimal optimization while preserving on-target editing, and they can be multiplexed to suppress several off-targets simultaneously. The disclosed dRNAs can be combined with high-specificity Cas9 variants, which often do not eliminate all unwanted editing. Moreover, the disclosed dRNAs can prevent cleavage of homology-directed repair (HDR)-corrected sites, facilitating “scarless” editing by eliminating the need for blocking mutations. The disclosed dRNAs thus facilitate more precise genome editing by establishing a flexible approach for suppressing unwanted editing of both off-target sequences and HDR-corrected sites.

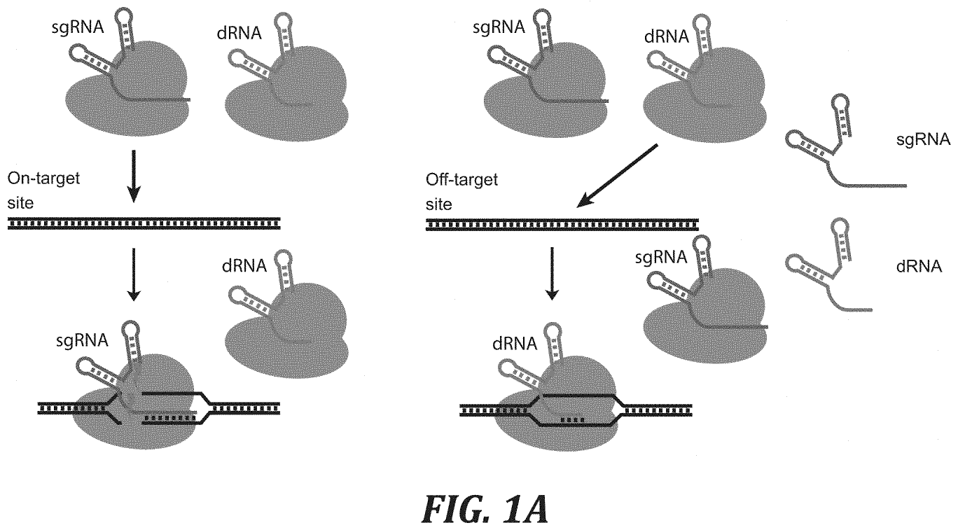

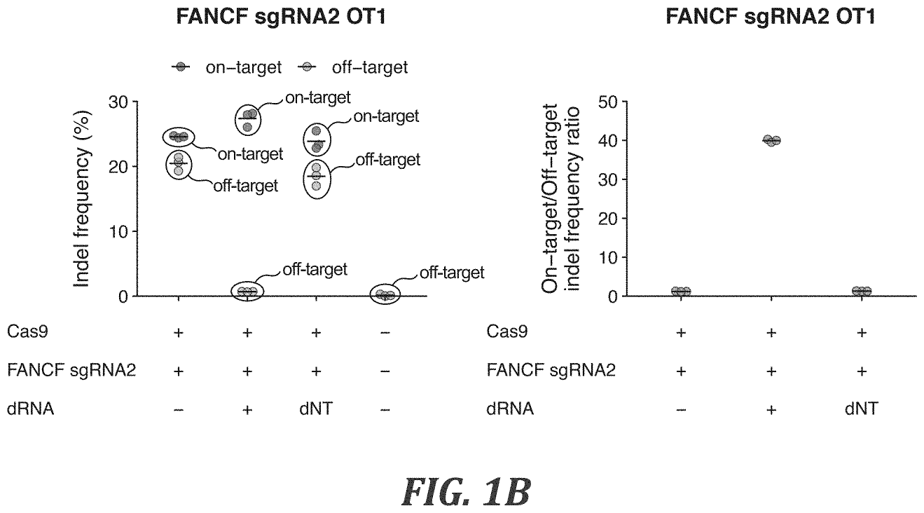

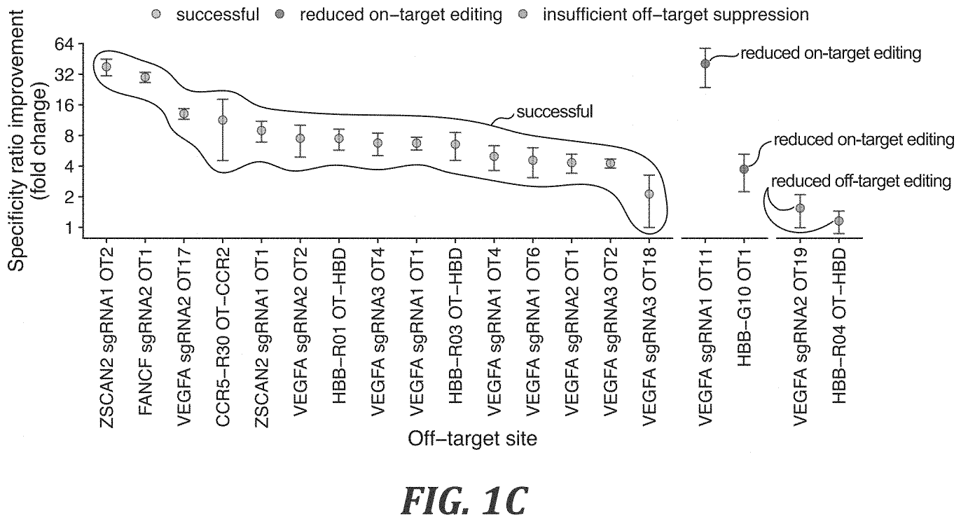

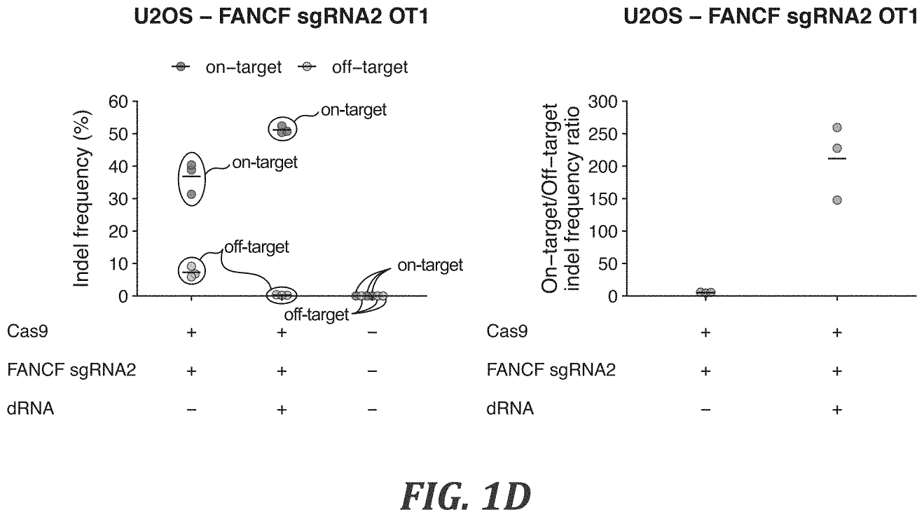

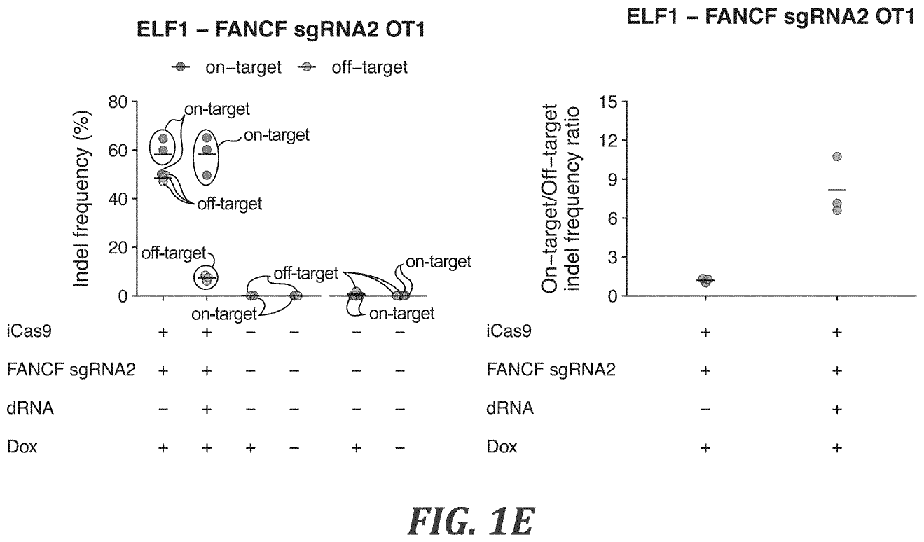

More specifically, the disclosed off-target suppression approach is based on the observation that sgRNAs with target recognition sequences 16 or fewer bases in length direct Cas9 binding to DNA target sites but do not promote cleavage. As described in more detail below, Cas9 bound to dRNAs with perfect complementarity to off-target sites can dramatically improve editing specificity by shielding these sites from the active Cas9•sgRNA complex (see FIG. 1A). To highlight the generality and ease of implementation of the disclosed method, which is also referred to as “dRNA Off-Target Suppression” (dOTS), editing was effectively suppressed at 15 off-target sites, yielding up to a ˜40-fold increase in specificity, with minimal dRNA optimization. Furthermore, it was demonstrated that dOTS can be multiplexed to suppress several off-targets simultaneously and can be combined with other approaches for improving specificity. Also described in more detail below is a method referred to as “dRNA ReCutting Suppression” (dReCS), wherein dRNAs prevent recutting of homology-directed repair (HDR)-corrected sites. The disclosed dReCS approach eliminates the need for blocking mutations and, thus, facilitates “scarless” editing. Thus, the disclosure provides more precise genome editing by establishing a novel and flexible approach for suppressing unwanted editing of both off-target and HDR-corrected sites.

In accordance with the foregoing, in one aspect the present disclosure provides a method of inhibiting off-target cleavage of a DNA molecule by a first guide RNA-endonuclease complex, wherein the first guide RNA-endonuclease complex comprises a first guide RNA comprising a nucleotide target recognition sequence complementary to a first target DNA sequence. The method comprises contacting the DNA molecule with a second guide RNA-endonuclease complex, wherein second guide RNA-endonuclease complex comprises a second guide RNA comprising a nucleotide target recognition sequence with 16 or fewer nucleotides and is complementary to a second target sequence in the DNA molecule. The second target sequence can be an off-target sequence at a different locus from the first target sequence. Such embodiments of the method can be referred to as “dRNA Off-Target Suppression” (dOTS). Alternatively, the second target sequence can be a different sequence that is obtained after editing of the first target sequence, i.e., at the same locus. Such embodiments of the method can be referred to as “dRNA ReCutting Suppression” (dReCS), which are discussed in more detail in another aspect below. The second target sequence is different from the first target sequence, but the second target sequence is capable of cleavage at a measurable rate by the first guide RNA-endonuclease complex, which is an activity referred to as off-target editing or off-target cutting.

As used herein, the term “inhibiting off-target cleavage” refers to the effect of reducing, limiting, slowing, or even preventing cleavage of a DNA molecule by a guide RNA-endonuclease complex at a sequence that has a slight sequence variation from the primary sequence targeted by the guide RNA-endonuclease complex. Typically, the primary target sequence is recognized by virtue of hybridization by a complementary sequence (i.e., the target recognition sequence) of the guide RNA. However, the target recognition sequence can also periodically hybridize to other sequences (e.g., at off-target sites) that have a slight sequence variation from the primary target site, leading to unintended modification on the DNA molecule. This phenomenon is inhibited or reduced by aspects of the present disclosure, leading to more accurate intended modifications. In some embodiments, contacting the DNA molecule with the second guide RNA-endonuclease complex, as described herein, reduces cleavage of the second target sequence by the first guide RNA-endonuclease complex by a measurable amount, for example at least about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, and about 100% compared to similar reaction conditions but wherein no second guide RNA-endonuclease complex is present. In some embodiments, contacting the DNA molecule with the second guide RNA-endonuclease complex prevents measurable cleavage of the second target sequence by the first guide RNA-endonuclease complex.

As indicated above, the target recognition sequence of the second guide RNA is configured to hybridize to a second target sequence (e.g., an “off-target” sequence) that is susceptible to potential cleavage by the first guide RNA-endonuclease complex. The second target sequence can be on the same DNA molecule as the first target DNA sequence. Alternatively, the second target sequence is on a different DNA molecule as the first target DNA sequence, but the two DNA molecules can co-exist in the same reaction environment (in vitro or in a cell). Typically, the second target sequence has some degree of sequence variation compared to the first target sequence. In some embodiments, the first and second target sequences are at distinct loci on the DNA molecule (or on different DNA molecules that exist in the same environment, such as a cell). In some embodiments, the first and second target sequences are at the same locus but represent the sequence before and after a genetic modification. In some embodiments, the first target sequence overlaps with, but is not completely encompassed by, the second target sequence. In some embodiments, the second target sequence differs from the first target sequence by 0-10 nucleotide mismatches. The indication of zero nucleotide mismatches refers to instances of incomplete overlap or presence of indels, as indicated above. However, in the region of overlap, there may be no mismatches at all. In other embodiments, in the aligned sequences there are mismatches, e.g. 1, 2, 3, 4, 5, 6, 7, 8, 9, and 10 mismatches. There have been reports of off-target editing at secondary sites with up to, e.g., 6, 7, and 9 mismatches.

In addition to the target recognition sequences, the guide RNAs referred to herein (e.g., first, second, and/or additional guide RNAs in multi-plexed reactions) can contain additional components that do not hybridize to a target DNA sequence through Watson and Crick base pairing. Instead, such other domain(s) can include a tracrRNA domain that interacts with a nuclease. Typically, tracrRNA domains interacting with endonucleases, e.g., Cas, have a stem-loop structure that facilitates complexing with the endonuclease. The tracrRNA and/or other scaffold domains are typically towards the 3′ end of the guide RNA molecule with the target recognition sequence being at the 5′ end of the guide RNA molecule.

In some embodiments, the endonuclease of the first guide RNA-endonuclease complex and/or the second guide RNA-endonuclease complex is a clustered regularly interspersed short palindromic repeats (CRISPR)/CRISPR-associated (Cas) system protein. Exemplary, non-limiting Cas endonucleases include Cas12a and Cas9. The present disclosure also encompasses variants or derivatives of Cas12a and Cas9. For example, in some embodiments the endonuclease is a high-fidelity variant of Cas9, such as eSpCas9, SpCas9-HF1, HypaCas9, as well as xCas9, SpCas9-NG, and the like. The endonucleases of the first guide RNA-endonuclease complex and the second guide RNA-endonuclease complex can be identical, variants of each other, or different specific endonucleases, such as selected from the non-limiting examples described above. The endonuclease of the first guide RNA-endonuclease complex and/or the second guide RNA-endonuclease complex can be derived from any source. Illustrative, non-limiting sources of appropriate RNA-guided endonucleases include Streptococcus, e.g., Streptococcus pyogenes, Staphylococcus, e.g., Staphylococcus aureus, Neisseria, e.g., Neisseria meningitidis, Acidaminococcus species, or Lachnospiraceae species.

The nucleotide target recognition sequence of the second guide RNA-endonuclease complex is sufficiently long to specifically hybridize to the second target sequence on the DNA molecule, but the length of the domain that hybridizes to the target DNA molecule does not exceed 16 nucleotides. By incorporating such a short domain for hybridization to the target sequence, the endonuclease complex with the guide RNA will not catalyze cleavage of the DNA molecule at the second target sequence. Accordingly, the second guide RNA is also referred to herein as a “dead” guide RNA, dead-RNA, or dRNA. In some embodiments, the nucleotide target recognition sequence of the second guide RNA-endonuclease complex comprises between 10 and 16 nucleotides, e.g., 10, 11, 12, 13, 14, 15, or 16 nucleotides, that are complementary to the second target sequence. In some embodiments, this shortened nucleotide target recognition sequence is completely complementary to the second target sequence on the target DNA (e.g., chromosomal DNA).

As indicated above, the first target sequence and the second target sequence can reside on the same DNA molecule or on different DNA molecules that are in the same reaction environment. For ease of explanation, potential steps of the method will be discussed in terms of “contacting the DNA molecule” that contains the second target sequence. However, it will be appreciated that this includes contacting a reaction environment that contains multiple DNA molecules where the first target sequence and the second target sequence reside on different DNA molecules, in addition to embodiments where the first target sequence and the second target sequence reside on the same DNA molecule. In some embodiments, the method further comprises contacting the DNA molecule, with or without other DNA molecules in the same reaction environment, with the first guide RNA-endonuclease complex. In some embodiments, the second guide RNA-endonuclease complex can be contacted to the DNA molecule prior to or simultaneously with the first guide RNA-endonuclease complex.

Multiple copies of first guide RNA-endonuclease complex and the second guide RNA-endonuclease complex can be contacted to the DNA molecule (or multiple copies of the DNA molecule) in various proportions. For example, the first guide RNA-endonuclease complex and the second guide RNA-endonuclease complex can be contacted to the DNA molecule at a ratio of about 20:1 to about 1:20, or any sub-range therein, such as about 15:1 to about 1:15, about 10:1 to about 1:10, about 5:1 to about 1:5, and about 2:1 to about 1:2. Exemplary ratios of the first guide RNA-endonuclease complex and the second guide RNA-endonuclease complex include 19:1, 18:1, 17:1, 16:1, 15:1, 14:1, 13:1, 12:1, 11:1, 10:1, 9:1, 8:1, 7:1, 6:1, 5:1, 4:1, 3:1, 2:1, 1:1, 1:2, 1:3, 1:4, 1:5, 1:6, 1:7, 1:8, 1:9, 1:10, 1:11, 1:12, 1:13, 1:14, 1:15, 1:16, 1:17, 1:18, and 1:19. The proportions can be adjusted according to persons of ordinary skill in the art to skew the performance of the reaction toward prioritizing the inhibition off-target editing by the first guide RNA-endonuclease complex or toward prioritizing modification of the first target sequence by the first guide RNA-endonuclease complex. A reaction that prioritizes the inhibition of off-target editing by the first guide RNA-endonuclease complex may have a relatively higher proportion of second guide RNA-endonuclease complex to the first guide RNA-endonuclease complex. Conversely, a reaction that prioritizes modification of the first target sequence by the first guide RNA-endonuclease complex may have a relatively higher proportion of first guide RNA-endonuclease complex to the second guide RNA-endonuclease complex. A person of ordinary skill in the art can readily determine the optimized ratio of the first guide RNA-endonuclease complex and the second guide RNA-endonuclease complex to achieve the preferred performance, taking into consideration the reaction conditions, sequences of the first and second target sequences, and performance of the RNA-endonuclease complexes, and the like.

The DNA molecule can be in a reaction environment within a cell (in vivo or in culture) or in an in vitro acellular reaction.

In some embodiments, the DNA molecule is in a cell and the step of contacting the DNA molecule with the second guide RNA-endonuclease complex comprises contacting the cell with one or more exogenous nucleic acid molecules comprising a first sequence encoding the second guide RNA and a second sequence encoding a second endonuclease. Upon expression of the first sequence and the second sequence the expressed second guide RNA and the expressed second endonuclease form the second guide RNA-endonuclease complex in the cell. As indicated above, in some embodiments, the method further comprises contacting the DNA molecule (and/or a reaction environment such as a cell containing the DNA molecule among others) with the first guide RNA-endonuclease complex. In this context, the one or more exogenous nucleic acid molecules further comprise a sequence encoding the first guide RNA. In some embodiments, only one sequence-type (referred to above as the “second sequence”) is provided that encodes an endonuclease (referred to above as the “second endonuclease”) and the multiple endonucleases expressed from the sequence independently associate with the first guide RNA and second guide RNA to form both the first RNA-endonuclease complex and the second guide RNA-endonuclease complex in the cell. Stated otherwise, the endonuclease(s) of the first RNA-endonuclease complex and the nuclease(s) second guide RNA-endonuclease complex are identical molecules encoded by the same sequence. In other embodiments, the one or more exogenous nucleic acid molecules comprise an additional sequence encoding a first endonuclease, as distinct from the encoded second endonuclease, such that the expressed first endonuclease associates with the expressed first guide RNA and the expressed second endonuclease associates with the expressed second guide RNA.

The exogenous nucleic acid molecules can be transferred into the cells by various methods including via vector, e.g., viral vectors such as retroviral or lentiviral vectors, plasmid vectors, transduction, transposons, chemical/lipid-mediated transfection, and electroporation. In some embodiments, the exogenous nucleic acid molecules are comprised in one or more vectors, e.g., viral expression vector, which facilitates expression of the heterologous nucleic acid in the nucleus of the cell. In some embodiments, the vector promotes integration of the heterologous nucleic acid in the genome of the cell.

A “vector” is a nucleic acid molecule that is capable of transporting another nucleic acid molecule. Vectors may be, for example, plasmids, cosmids, viruses, an RNA vector or a linear or circular DNA or RNA molecule that may include chromosomal, non-chromosomal, semi-synthetic or synthetic nucleic acid molecules. Exemplary vectors are those capable of autonomous replication (episomal vector) or expression of nucleic acid molecules to which they are linked (expression vectors). The vector may be a plasmid, a phage particle, a virus, or simply a potential genomic insert. Once transformed into a suitable host cell, the vector may replicate and function independently of the host genome, or may, in some instances, integrate into the genome itself. In the present specification, “plasmid,” “expression plasmid,” “virus” and “vector” are often used interchangeably. The vectors can be configured to promote integration of the encoding sequences into one or more DNA molecules in the reaction environment (e.g., into one or more chromosomes of the cells), or to promote expression of the encoding sequences directly from the vectors.

The encoding sequences can be integrated into expression cassettes that also include control sequences operatively linked to the encoding sequence that promote transcription. The term “operably linked” refers to the association of two or more nucleic acid sequences or domains on a single nucleic acid fragment so that the function of one is affected by the other. For example, a promoter is operably linked with a coding sequence when it is capable of affecting the expression of that coding sequence (i.e., the coding sequence is under the transcriptional control of the promoter). The control sequences include a promoter to effect transcription, an optional operator sequence to control such transcription, a sequence encoding suitable mRNA ribosome binding sites, and sequences which control termination of transcription. Useful promoter sequences that, e.g., facilitate assembly and activation of the requisite gene expression factors, are known. Promoters can facilitate transient or constitutive transcription of the encoding sequences. The one or more exogenous DNA molecules can be integrated into the same vector, different vectors of the same type, or different vectors of different types.

Viral vectors encompassed by the disclosure include retrovirus, adenovirus, parvovirus (e.g., adeno-associated viruses), coronavirus, negative strand RNA viruses such as ortho-myxovirus (e.g., influenza virus), rhabdovirus (e.g., rabies and vesicular stomatitis virus), paramyxovirus (e.g., measles and Sendai), positive strand RNA viruses such as picornavirus and alphavirus, and double-stranded DNA viruses including adenovirus, herpesvirus (e.g., Herpes Simplex virus types 1 and 2, Epstein-Barr virus, cytomegalovirus), and poxvirus (e.g., vaccinia, fowlpox and canarypox). Other viruses include Norwalk virus, togavirus, flavivirus, reoviruses, papovavirus, hepadnavirus, and hepatitis virus, for example. Examples of retroviruses include avian leukosis-sarcoma, mammalian C-type, B-type viruses, D type viruses, HTLV-BLV group, lentivirus, spumavirus (Coffin, J. M., Retroviridae: The viruses and their replication, In Fundamental Virology, Third Edition, B. N. Fields et al., Eds., Lippincott-Raven Publishers, Philadelphia, 1996). “Lentiviral vector,” as used herein, means HIV-based lentiviral vectors for gene delivery, which can be integrative or non-integrative, have relatively large packaging capacity, and can transduce a range of different cell types. Lentiviral vectors are usually generated following transient transfection of three (packaging, envelope and transfer) or more plasmids into producer cells. Like HIV, lentiviral vectors enter the target cell through the interaction of viral surface glycoproteins with receptors on the cell surface. On entry, the viral RNA undergoes reverse transcription, which is mediated by the viral reverse transcriptase complex. The product of reverse transcription is a double-stranded linear viral DNA, which is the substrate for viral integration into the DNA of infected cells.

In other embodiments, the step of contacting the DNA molecule with the second guide RNA-endonuclease complex, whether in a cell or in an acellular reaction environment, comprises contacting the cell or reaction environment with a pre-assembled second guide RNA-endonuclease complex. The pre-assembled second guide RNA-endonuclease complex can be generated in a cell culture line. Exemplary methods for generating the guide RNA-endonuclease complex that are applicable to this embodiment are described in more detail in the Example section below.

In a further embodiment, the method can further comprise contacting the DNA molecule, or a reaction environment (e.g., cell or in vitro reaction mix) with a pre-assembled first guide RNA-endonuclease complex.

In additional embodiments, especially wherein the DNA molecule is in a cell, one of the first guide RNA-endonuclease complex and second guide RNA-endonuclease complex is contacted to the DNA molecule by contacting the cell with the one or more exogenous nucleic acid molecules that encode the guide RNA and respective endonuclease. The nucleic acid molecules are allowed be transcribed in the cell resulting in the formation of the guide RNA and the endonuclease, which form the complex, as described in more detail above. The other of the first guide RNA-endonuclease complex and second guide RNA-endonuclease complex can be contacted to the DNA molecule in a pre-assembled form, as described above.

Delivery of the pre-assembled first guide RNA-endonuclease complex and/or second guide RNA-endonuclease complex can be facilitated by techniques or delivery vehicles that promote intra-cellular delivery of macromolecules. For example, the delivery technique can include electroporation, or chemical/lipid-mediated transfection, accordingly routine skill and knowledge of practitioners in the art.

The discussion has heretofore been in the context of inhibiting off-target editing by a first guide RNA-endonuclease complex incorporating use of a second guide RNA-endonuclease complex to prevent editing at the second target second on the DNA molecule. However, as described below, it was established that use of dead-RNA as guide RNA molecules to inhibit off-target editing (i.e., by the first guide RNA-endonuclease complex) can be multiplexed to inhibit off-target editing by the first guide RNA-endonuclease complex at multiple, distinct off-target sites on one or more DNA molecules in the same reaction environment. Accordingly, this disclosure encompasses multiplexing with two or more additional guide RNA-endonuclease complexes (i.e., multiple “second guide RNA-endonuclease complexes” with distinct dead guide RNA components specific for different off-target sequences). In such multiplexed embodiments, each of the two or more additional complexes comprises a different nucleotide target recognition sequence with 16 or fewer nucleotides and corresponds to (e.g., is complementary to) a distinct target site in the target DNA molecule or another DNA molecule in the same reaction environment. The two or more additional target sequences are different from each other and from the first target sequence but are each capable of cleavage at measurable rates by the first guide RNA-endonuclease complex. The multiplex reaction can comprise the second guide RNA-endonuclease, as described above, in addition to 1, 2, 3, 4, 5, 6, 7, 8, 9, 10, or more additional dead guide RNA-endonuclease complexes, as generally described above.

Additionally, as described below, it was established that multiple distinct target sites (i.e., multiple distinct “first target sequences”) can be cleaved in a single multiplexed reaction that includes multiple guide RNA-endonuclease complexes with distinct but functional guide RNAs (i.e., multiple “first guide RNA-endonuclease complexes” with distinct guide RNA components) specific for different target sequences. In such a multiplexed reaction each distinct “first guide RNA-endonuclease complex” is accompanied by at least one corresponding “second guide RNA-endonuclease complex” with a dead guide RNA component that targets an off-target (or “second”) target sequence associated with the first guide RNA-endonuclease complex. Accordingly, the disclosure also encompasses multiplexed reactions where multiple and distinct sequences are targeted for modification in the same reaction that also contains multiple dead “second” guide RNA-endonuclease complexes blocking off-target cleavage by the different “first” guide RNA-endonuclease sequences.

As indicated above, the dead guide RNAs were integrated into a strategy, referred to as “dRNA ReCutting Suppression” (dReCS), to prevent recutting of an edited site. In the context of the discussion above, the initial pre-edited sequence of a locus can be considered the first target sequence. After cutting and editing by homology-directed repair (HDR), the locus sequence becomes the second target sequence that may be susceptible to “off-target” cutting by the initial guide RNA-endonuclease complex. Inclusion of a second dead-RNA-endonuclease complex can then shield the new sequence from recutting by the initial guide RNA-endonuclease that remains in the environment without requiring further addition of blocking mutations. This is essentially a “scarless” editing technique that allows for more efficient genetic editing.

Accordingly, in another aspect the disclosure provides a method of inhibiting cleavage of a DNA molecule at a target site that has been previously modified from containing a first sequence to containing a second sequence. For example, this modification can be implemented by targeted cleavage by a first guide RNA-endonuclease complex and subsequent homology-directed repair (HDR), wherein the first guide RNA-endonuclease complex comprises a nucleotide target recognition sequence complementary to the first sequence. In this scenario, the method can be referred to as “scarless HDR.” However, this aspect also encompasses other strategies and mechanisms for implementing the initial modification from the first sequence to the second sequence.

As will be appreciated by persons of ordinary skill in the art, the requirement to permit scarless editing according to this aspect is that the second guide RNA-endonuclease complex anneal to the edited locus (i.e., at the second sequence) and provide steric hindrance to inhibit or reduce the likelihood of re-cutting at the site. In this context, the term “inhibiting” refers to the reduction of cleavage at the target site that has been edited from a first sequence to a second sequence. In some embodiments, contacting the DNA molecule with the second guide RNA-endonuclease complex, as described herein, reduces cleavage of the second target sequence by the first guide RNA-endonuclease complex, or otherwise by another homology repair mechanism, by a measurable amount, such as by at least about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, and about 100% compared to similar reaction conditions but wherein no second guide RNA-endonuclease complex is present. In some embodiments, contacting the DNA molecule with the second guide RNA-endonuclease complex prevents measurable cleavage of the second target sequence by the first guide RNA-endonuclease complex. In some embodiments, prevention of measurable cleavage of the second target sequence by the first guide RNA-endonuclease complex is determinable by characterizing the sequence of the modified DNA molecule to confirm the intended second sequence rather than an aberrant, unintended sequence resulting from further off-target cleavage of the second sequence.

In some embodiments, the second sequence differs from the first sequence by 0-10 nucleotide mismatches. The indication of zero nucleotide mismatches refers to instances of incomplete overlap or presence of indels, as indicated above. However, in the region of overlap, there may be no mismatches at all. In other embodiments, in the aligned sequences there are mismatches, e.g. 1, 2, 3, 4, 5, 6, 7, 8, 9, and 10 mismatches. There have been reports of off-target editing at secondary sites with up to, e.g., 6, 7, and 9 mismatches.

The method comprises contacting the DNA molecule with a second guide RNA-endonuclease complex, wherein the guide RNA of the second guide RNA-endonuclease complex comprises a second guide RNA comprising a nucleotide target recognition sequence with 16 or fewer nucleotides and is complementary to at least a portion of the second sequence in the DNA molecule. The second sequence is different from the first sequence but the second sequence is capable of cleavage at a measurable rate by the first guide RNA-endonuclease complex. In the context of the above description of the prior aspect, the second sequence is equivalent to an off-target sequence relative to the first sequence, although they both are at the same locus post and pre-editing, respectively.

In some embodiments, the method further comprises inducing targeted cleavage of the DNA molecule containing the first sequence by contacting the DNA molecule with the first guide RNA-endonuclease complex, thereby producing a cleaved DNA molecule. The method can even further comprise contacting the cleaved DNA molecule with a repair polynucleotide that is substantially homologous to the target site but comprises the second sequence. Hence, the cleaved DNA molecule will be repaired and altered by homology-directed repair (HDR).

Multiple copies of first guide RNA-endonuclease complex and the second guide RNA-endonuclease complex can be contacted to the DNA molecule (or multiple copies of the DNA molecule) in various proportions. For example, the first guide RNA-endonuclease complex and the second guide RNA-endonuclease complex can be contacted to the DNA molecule at a ratio of about 20:1 to about 1:20, or any sub-range therein, as described in more detail above.

Structural elements of the first guide RNA-endonuclease and the second guide RNA-endonuclease complexes include the elements discussed in more detail above with respect to the first aspect of the disclosure.

In some embodiments, the endonuclease of the first guide RNA-endonuclease complex and/or the second guide RNA-endonuclease complex is a clustered regularly interspersed short palindromic repeats (CRISPR)/CRISPR-associated (Cas) system protein. Exemplary, non-limiting Cas endonucleases include Cas12a and Cas9. The present disclosure also encompasses variants or derivatives of Cas12a and Cas9. For example, in some embodiments the endonuclease is a high-fidelity variant of Cas9, such as eSpCas9, SpCas9-HF1, HypaCas9, as well as xCas9, SpCas9-NG, and the like. The endonucleases of the first guide RNA-endonuclease complex and the second guide RNA-endonuclease complex can be identical, variants of each other, or different specific endonucleases, such as selected from the non-limiting examples described above. The endonuclease of the first guide RNA-endonuclease complex and/or the second guide RNA-endonuclease complex can be derived from any source. Illustrative, non-limiting sources of appropriate RNA-guided endonucleases include Streptococcus, e.g., Streptococcus pyogenes, Staphylococcus, e.g., Staphylococcus aureus, Neisseria, e.g., Neisseria meningitidis, Acidaminococcus species, or Lachnospiraceae species.

The nucleotide target recognition sequence of the second guide RNA-endonuclease complex is sufficiently long to specifically hybridize to the second sequence on the DNA molecule, but the length of the domain that hybridizes to the target DNA molecule does not exceed 16 nucleotides. By incorporating such a short domain for hybridization to the target sequence, this second endonuclease complex with the guide RNA will not catalyze cleavage of the target DNA molecule. In some embodiments, the nucleotide target recognition sequence of the second guide RNA-endonuclease complex comprises between, e.g., 10, 11, 12, 13, 14, 15, or 16 nucleotides that are complementary to the second target sequence. In some embodiments, this shortened nucleotide recognition sequence is completely complementary to the second sequence on the target DNA (e.g., chromosomal DNA).

The DNA molecule can be in a cell (e.g., in vivo or an ex vivo culture) or in an acellular reaction. As described in more detail above, the step of contacting the DNA molecule with the second guide RNA-endonuclease complex, and optional also with the guide RNA-endonuclease complex, can comprise contacting the cell containing the DNA molecule with one or more exogenous nucleic acid molecules comprising a sequence(s) encoding the guide RNA(s) (e.g., one or both of the first and second guide RNAs) and a second sequence encoding an endonuclease, and optionally a third sequence encoding another, distinct endonuclease.

The one or exogenous nucleic acids can be integrated into a vector, as described in more detail above.

Alternatively, the step of contacting the DNA molecule with the second guide RNA-endonuclease complex, and optionally with the guide RNA-endonuclease complex, can comprise contacting the cell or other environment containing the DNA molecule with a pre-assembled second guide RNA-endonuclease complex, and optionally a pre-assembled first guide RNA-endonuclease complex.

In additional embodiments, especially wherein the DNA molecule is in a cell, one of the first guide RNA-endonuclease complex and second guide RNA-endonuclease complex is contacted to the DNA molecule by contacting the cell with the one or more exogenous nucleic acid molecules that encode the guide RNA and respective endonuclease. The nucleic acid molecules are allowed be transcribed in the cell resulting in the formation of the guide RNA and the endonuclease, which form the complex, as described in more detail above. The other of the first guide RNA-endonuclease complex and second guide RNA-endonuclease complex can be contacted to the DNA molecule in a pre-assembled form, as described above.

In other aspects, the disclosure provides compositions comprising or providing endonucleases complexed with truncated, or “dead-RNAs” (dRNAs), that render the endonuclease unable to cleave target DNA but still allow hybridization of the complex to the target DNA.

In one aspect, the disclosure provides a composition comprising a first guide RNA-endonuclease complex and a second guide RNA-endonuclease complex. The guide RNA of the first guide RNA-endonuclease complex comprises a nucleotide target recognition sequence complementary to a first target sequence in a DNA molecule, and the guide RNA of the second guide RNA-endonuclease complex comprises a nucleotide target recognition sequence with 16 or fewer nucleotides and is complementary to a second target site in the DNA molecule or in a distinct DNA molecule. The second target sequence is different from the first target sequence but the second target sequence is capable of cleavage at a measurable rate by the first guide RNA-endonuclease complex. Other elements of the first and second guide RNA-endonuclease complexes are described in more detail above and are encompassed by this aspect.

The composition can be formulated for administration to a subject or to cell cultures as appropriate according to techniques known in the art.

In another aspect, the disclosure provides a plasmid or vector that encodes the elements of the first and second guide RNA-endonuclease complexes that are described in more detail above. For example, the plasmid or vector comprises nucleic acid domains encoding a first guide RNA, a second guide RNA, and an endonuclease each operatively linked to a promoter sequence. The first guide RNA comprises a nucleotide target recognition sequence complementary to a first target sequence and the second guide RNA comprises a nucleotide target recognition sequence with 16 or fewer nucleotides and is complementary to a second target site. The second target sequence is different from the first target sequence but the second target sequence is capable of cleavage at a measurable rate by a complex of the first guide RNA and the endonuclease. The encoded first and second guide RNAs can contain additional components that do not hybridize to a target DNA sequence through Watson and Crick base pairing. Instead, such other domain(s) can include a tracrRNA domain that interacts with a nuclease, as described above. Other, non-coding elements typical of plasmids and vectors are also described in more detail above and are encompassed in this aspect.

In other aspects, the disclosure provides a kit incorporating various elements of the compositions described above.

In another aspect of the disclosure provides a kit. The kit comprises a first guide RNA-endonuclease complex and a second guide RNA-endonuclease complex. The guide RNA of the first guide RNA-endonuclease complex comprises a nucleotide target recognition sequence complementary to a first target sequence in a DNA molecule, and the guide RNA of the second guide RNA-endonuclease complex comprises a nucleotide target recognition sequence with 16 or fewer nucleotides and is complementary to a second target site in the DNA molecule. The second target sequence is different from the first target sequence but the second target sequence is capable of cleavage at a measurable rate by the first guide RNA-endonuclease complex. Structural elements of the first guide RNA-endonuclease complex and a second guide RNA-endonuclease complex are described in more detail above and are encompassed by embodiments of this aspect. The kit can further comprise written indicia for inhibiting off-target cleavage of a DNA molecule by the first guide RNA-endonuclease complex and/or for implementing HDR without inclusion additional mutations to block recutting by the first guide RNA-endonuclease.

In another aspect, the disclosure provides a kit comprising one of the following:

the plasmid or vector as described above; or

a plurality of vectors that, in aggregate, comprise nucleic acid domains encoding a first guide RNA, a second guide RNA, and at least one endonuclease that can form complexes with the encoded guide RNAs. Each nucleic acid is operatively linked to a promoter sequence within their respective vectors. In some embodiments, the nucleic acid domains encoding first and second guide RNAs are in the same vector. In some embodiments, one of the nucleic acid domains encoding the first and second guide RNAs is in the same vector with the nucleic acid encoding the endonuclease. In some embodiments, the nucleic acid domain encoding the first guide RNA and the nucleic acid domain encoding a first endonuclease are both incorporated into a first vector, and the nucleic acid domain encoding the second guide RNA and a nucleic acid domain encoding a second endonuclease are incorporated into a second vector. In yet a further embodiment, the nucleic acids encoding the first guide RNA, the second guide RNA, and the endonuclease(s) are each incorporated into separate vectors.

Regardless of configuration, the first guide RNA comprises a nucleotide target recognition sequence complementary to a first target sequence and the second guide RNA comprises a nucleotide target recognition sequence with 16 or fewer nucleotides and is complementary to a second target site. The second target sequence is different from the first target sequence but the second target sequence is capable of cleavage at a measurable rate by a complex of the first guide RNA and the endonuclease.

The kit can also comprise written indicia for reducing or preventing off-target cleavage of a DNA molecule by the first guide RNA-endonuclease complex and/or for implementing HDR without inclusion additional mutations to block recutting by the first guide RNA-endonuclease.

Unless specifically defined herein, all terms used herein have the same meaning as they would to one skilled in the art of the present disclosure. Practitioners are particularly directed to Ausubel, F. M., et al. (eds.), Current Protocols in Molecular Biology, John Wiley & Sons, New York (2010); Coligan, J. E., et al. (eds.), Modern Proteomics—Sample Preparation, Analysis and Practical Applications in Advances in Experimental Medicine and Biology, Springer International Publishing, 2016; Comai, L, et al., (eds.), Proteomic: Methods and Protocols in Methods in Molecular Biology, Springer International Publishing, 2017; and Komor, A. C., et al., CRISPR-based technologies for the manipulation of eukaryotic genomes. Cell, 168(1-2), 20-36 (2017), for definitions and terms of art. These references are specifically incorporated herein by reference.

For convenience, certain terms employed in the specification, examples, and appended claims are provided here. The definitions are provided to aid in describing particular embodiments and are not intended to limit the claimed invention, as the scope of the invention is limited only by the claims.

The use of the term “or” in the claims and specification is used to mean “and/or” unless explicitly indicated to refer to alternatives only or the alternatives are mutually exclusive, although the disclosure supports a definition that refers to only alternatives and “and/or.”

The words “a” and “an,” when used in conjunction with the word “comprising” in the claims or specification, denotes one or more, unless specifically noted.

Unless the context clearly requires otherwise, throughout the description and the claims, the words “comprise,” “comprising,” and the like, are to be construed in an open and inclusive sense as opposed to a closed, exclusive or exhaustive sense. For example, the term “comprising” can be read to indicate “including, but not limited to.” The term “consists essentially of” or grammatical variants thereof indicate that the recited subject matter can include additional elements not recited in the claim, but which do not materially affect the basic and novel characteristics of the claimed subject matter.

Words using the singular or plural number also include the plural and singular number, respectively. The word “about” indicates a number within range of minor variation above or below the stated reference number. For example, “about” can refer to a number within a range of 10%, 9%, 8%, 7%, 6%, 5%, 4%, 3%, 2%, or 1% above or below the indicated reference number.

The term “nucleic acid” refers to a deoxyribonucleotide or ribonucleotide polymer in either single- or double-stranded form, and unless otherwise limited, encompasses known analogs of natural nucleotides that hybridize to nucleic acids in manner similar to naturally occurring nucleotides. Unless otherwise indicated, a particular nucleic acid sequence includes the complementary sequence thereof.

Reference to sequence identity addresses the degree of similarity of two polymeric sequences, such as protein sequences or nucleic acid sequences. Determination of sequence identity can be readily accomplished by persons of ordinary skill in the art using accepted algorithms and/or techniques. Sequence identity is typically determined by comparing two optimally aligned sequences over a comparison window, where the portion of the peptide or polynucleotide sequence in the comparison window may comprise additions or deletions (i.e., gaps) as compared to the reference sequence (which does not comprise additions or deletions) for optimal alignment of the two sequences. The percentage is calculated by determining the number of positions at which the identical amino-acid residue or nucleic acid base occurs in both sequences to yield the number of matched positions, dividing the number of matched positions by the total number of positions in the window of comparison and multiplying the result by 100 to yield the percentage of sequence identity. Various software driven algorithms are readily available, such as BLAST N or BLAST P to perform such comparisons.

Disclosed are materials, compositions, and components that can be used for, can be used in conjunction with, can be used in preparation for, or are products of the disclosed methods and compositions. It is understood that, when combinations, subsets, interactions, groups, etc., of these materials are disclosed, each of various individual and collective combinations is specifically contemplated, even though specific reference to each and every single combination and permutation of these compounds may not be explicitly disclosed. This concept applies to all aspects of this disclosure including, but not limited to, steps in the described methods. Thus, specific elements of any foregoing embodiments can be combined or substituted for elements in other embodiments. For example, if there are a variety of additional steps that can be performed, it is understood that each of these additional steps can be performed with any specific method steps or combination of method steps of the disclosed methods, and that each such combination or subset of combinations is specifically contemplated and should be considered disclosed. Additionally, it is understood that the embodiments described herein can be implemented using any suitable material such as those described elsewhere herein or as known in the art.

Publications cited herein and the subject matter for which they are cited are hereby specifically incorporated by reference in their entireties.

### EXAMPLES

The following examples are set forth so as to provide those of ordinary skill in the art with a complete disclosure and description of how to make and use the present invention, and are not intended to limit the scope of what the inventors regard as their invention nor are they intended to represent that the experiments below are all or the only experiments performed.

### Example 1

The Example provides a description of the design and implementation of “dRNA Off-Target Suppression” (dOTS) and “dRNA ReCutting Suppression” (dReCS) techniques that leverage co-administration of catalytically inactive nuclease/guide RNA complexes that incorporate truncated guide RNAs directed to off target sites.

### Results

Dead-RNA Off-Target Suppression Increases On-Target Specificity

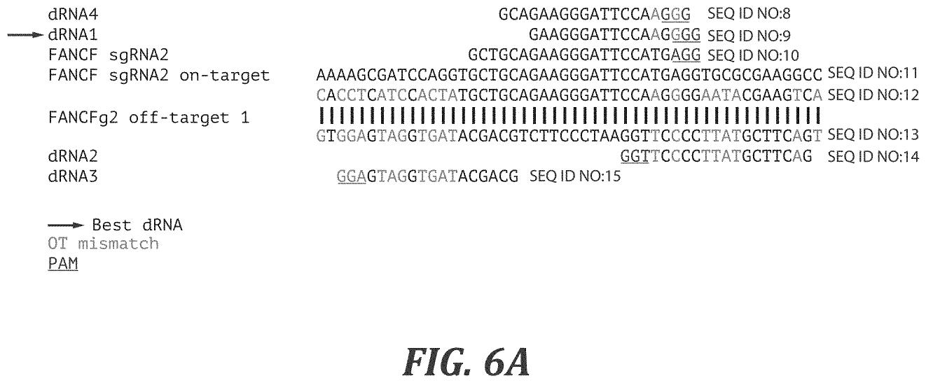

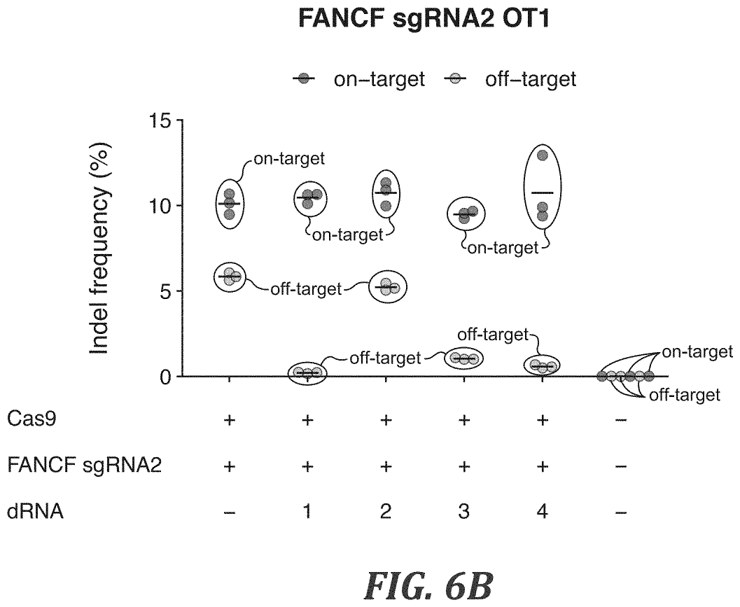

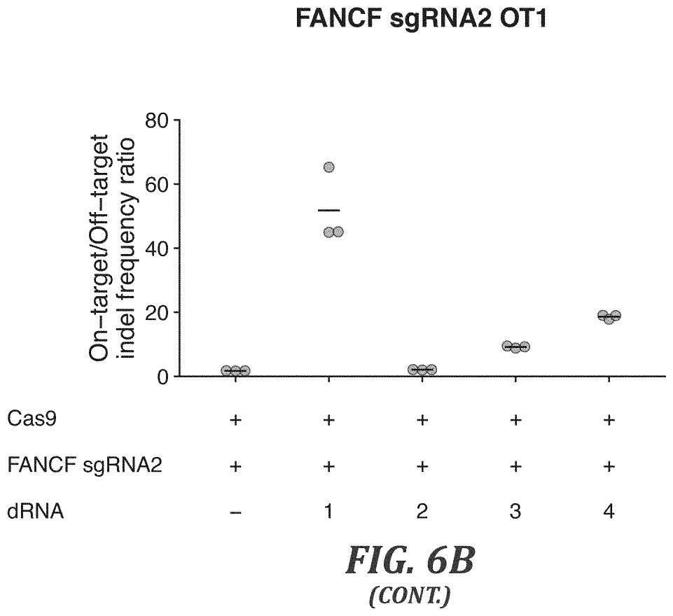

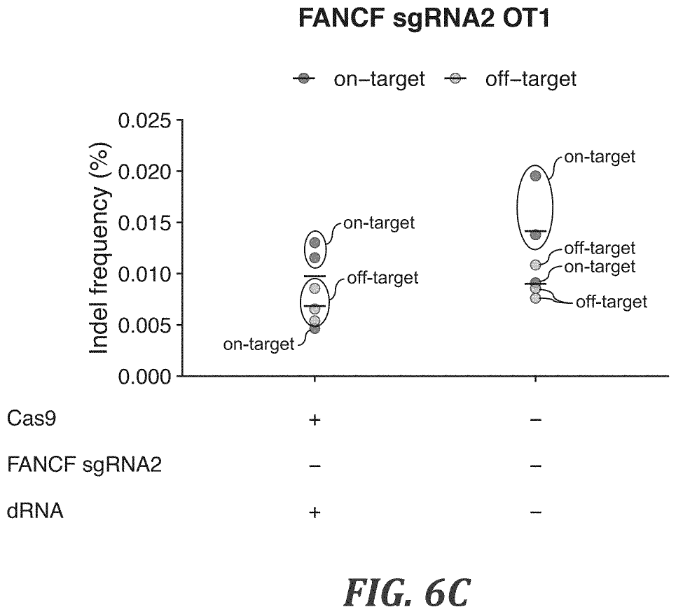

First, the feasibility of using dRNAs to suppress unwanted editing at off-target site 1 (OT1) of an sgRNA (sgRNA2) targeting the FANCF locus (Slaymaker, I. M., et al., Rationally engineered Cas9 nucleases with improved specificity. Science 351, 84-88 (2016), incorporated herein by reference in its entirety) was assessed. HEK-293T cells were co-transfected with a plasmid encoding SpCas9, along with equal amounts of plasmids encoding FANCF sgRNA2 and a GFP control, or FANCF sgRNA2 and one of four dRNAs with perfect complementarity to OT1 (FIG. 6A). Three of the four dRNAs significantly decreased off-target editing without appreciably impacting on-target editing, while co-transfection of a non-targeting control dRNA did not impact on- or off-target editing (FIG. 6B). In particular, dRNA1 decreased off-target editing from 20.44% (s.e.m.=0.61%, n=3) to 0.69% (s.e.m=0.02%, n=3), leading to a 30-fold increase in the on-target specificity ratio (FIG. 1B). Cas9•dRNA complexes are thought to lack cleavage activity, but only a relatively small number of dRNAs have been evaluated so far. Thus, it was verified that dRNA1 did not direct any detectable Cas9 editing activity at either the on- or off-target sites (FIG. 6C). Briefly, indel frequencies at the FANCF sgRNA2 on-target and OT1 sites were assessed at 24 hours after transfection with Cas9 and dRNA1 but without sgRNA, with the predicted cut sites of dRNA1 are the same as FANCF sgRNA2. It was further confirmed that dRNA1 showed no cleavage genome-wide using GUIDE-seq (Tsai, S. Q., et al., GUIDE-seq enables genome-wide profiling of off-target cleavage by CRISPR-Cas nucleases. Nature Biotechnology 33, 187-197 (2015), incorporated herein by reference in its entirety), and that it directed selective reduction of only OT1 (not shown). Briefly, indel frequency was assessed at FANCF sgRNA2 on-target site and OT1 96 hours after electroporation with plasmids encoding Cas9, sgRNA and dRNA1 in U2OS cells. Integration of an end-protected double-stranded oligonucleotide (dsODN) was assessed and expressed as integration efficiency an ratio (integration:indel) 96 hours after electroporation. Indel frequencies and dsODN tag integration for untransfected cells are shown as a control. GUIDE-seq genome-wide specificity profiles were generated for Cas9 paired with FANCF sgRNA2, FANCF dRNA1 or both allowing for up to 8 mismatches from on-target sequence and GUIDE-seq counts and frequencies were generated (not shown). This experiment is the first known demonstration that a dRNA leads to no detectable cleavage activity anywhere in the genome.

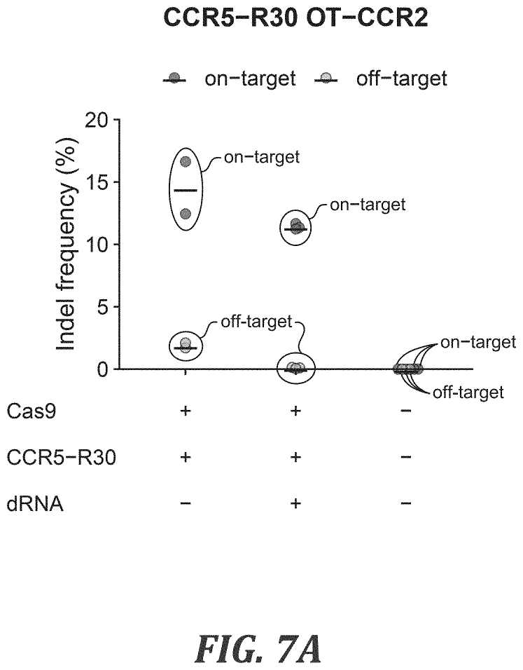

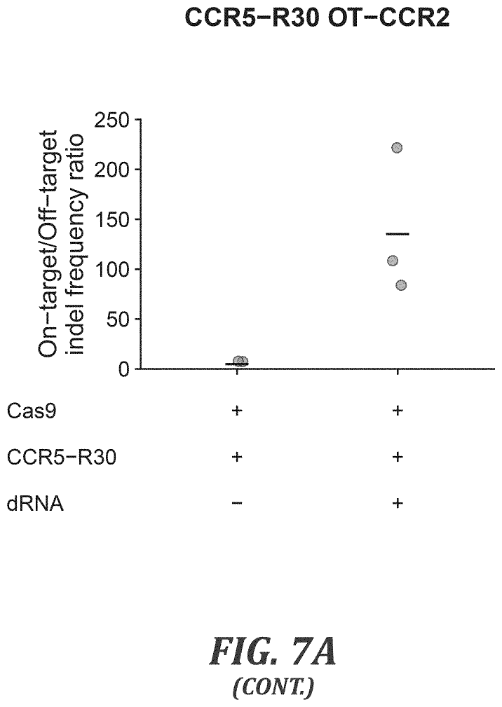

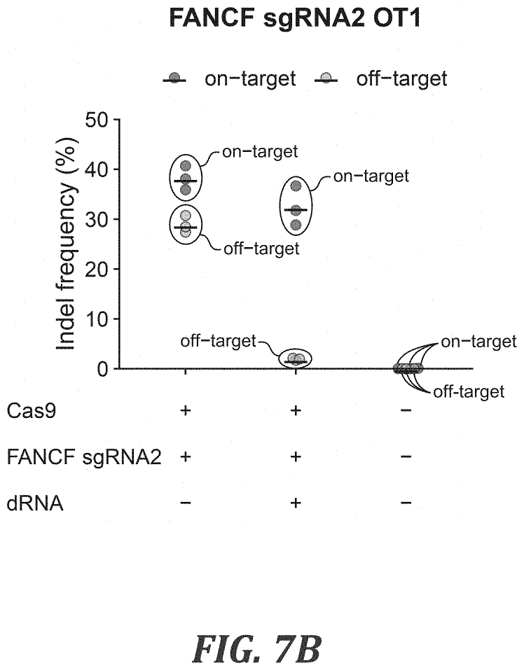

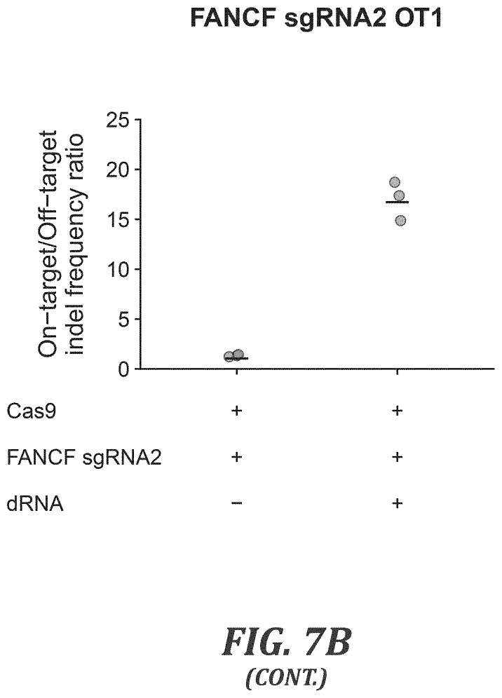

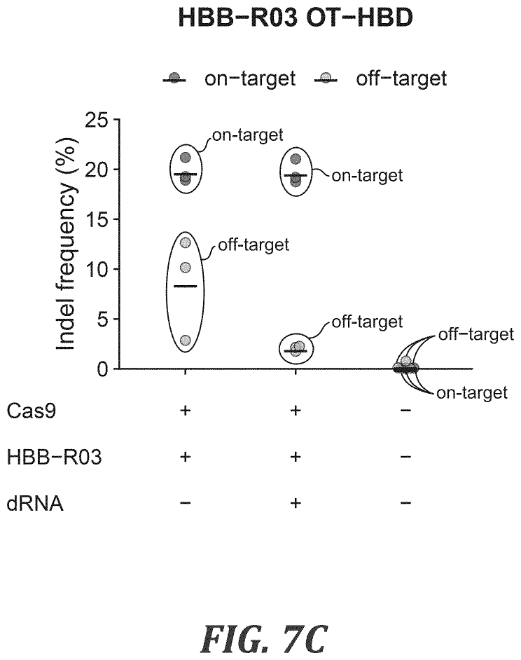

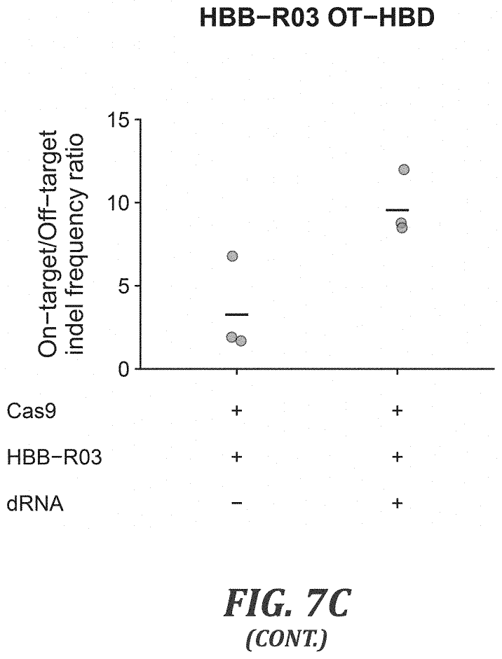

To demonstrate the generality of dOTS, 19 on-target/off-target pairs were evaluated in HEK-293T cells. Briefly, on-target/off-target indel frequencies were assessed 24 hours after transfection with Cas9, sgRNA, and off-target specific dRNAs in HEK293T cells. The assessed parings were: FANCF OT1, CCR5-R30 OT (CCR2), HBB-G10 OT1, HBB-R01 OT (HBD), HBB-R03 OT (HBD), HBB-R04 OT (HBD), VEGFA sgRNA1 OT1, VEGFA sgRNA1 OT4, VEGFA sgRNA1 OT6, VEGFA sgRNA1 OT11, VEGFA sgRNA2 OT1, VEGFA sgRNA2 OT2. (1) VEGFA sgRNA2 OT17, VEGFA sgRNA2 OT19, VEGFA sgRNA2 OT19, VEGFA sgRNA3 OT2, VEGFA sgRNA3 OT4, VEGFA sgRNA3 OT18, ZSCAN2 sgRNA1 OT1, and ZSCAN2 sgRNA1 OT2. Indel frequencies for untransfected cells were used as a control. At least one dRNA was found for 15 of the 19 pairs that were tested that increased the specificity ratio by at least two-fold (mean fold-change=10.44) while decreasing on-target editing by no more than two-fold (mean fold-change=0.93; FIG. 1C). Across all on-target/off-target pairs, a median of six candidate dRNAs were screened, highlighting the ease of identifying effective dRNAs (TABLE 1). In most cases, non-targeting dRNAs had little to no impact on editing. This was determined by comparing the most effective dRNA for 12 different off-target loci with a nontargeting dRNA (dNT). Indel frequency of on-target and off-target loci was observed at 24 hours after transfection with Cas9, sgRNA, ±dRNA or nontargeting dRNA in HEK293T cells. The target loci pairs were: HBB R03 OT-HBD, VEGFA sgRNA1 OT1, VEGFA sgRNA1 OT6, VEGFA sgRNA2 OT1, VEGFA sgRNA2 OT2, VEGFA sgRNA2 OT17, VEGFA sgRNA3 OT2, VEGFA sgRNA3 OT4, VEGFA sgRNA3OT18, ZSCAN2 sgRNA1 OT1, and ZSCAN2 sgRNA1 OT2 (not shown). Moreover, effective dRNAs did not induce indels at either on- or off-target sites, suggesting that few, if any, Cas9•dRNA complexes are active (TABLES 2 and 3). dOTS was also as effective in U2OS cells and the Elf1 naïve embryonic stem cell line as in HEK-293T cells (FIGS. 1D and 1E). Similar results were also observed using alternative pairings of VEGFA sgRNA3 OT2 in U2OS cells and HBB-R03 OT-HBD in ELF1 cells (not shown). Finally, it was confirmed that dRNA-mediated suppression of off-target editing was durable, with dRNAs effectively decreasing off-target editing for at least 72 hours post-transfection (FIGS. 7A-7C).

TABLE 1
dRNAs designed for a variety of sites increase specificity ratio with minimal effects
on on-target editing. Normalized specificity ratios, computed as the specificity ratio
in the presence of the best dRNA at a site divided by the specificity ratio in the
absence of the dRNA, and on-target ratios, computed as the ratio of on-target editing
in the presence of the best dRNA at a site divided by the on-target editing in the
absence of the dRNA, for the best dRNA for 19 sgRNA/off-target pairs. n = 3 biological
replicates, error measured as the standard error of the mean (s.e.m.).

| | | | Normalized | | Normalized | |
|---|---|---|---|---|---|---|
| | | | specificity | On-target | specificity | On-target |
| | | | ratio | ratio | ratio | ratio |
| Site | Best dRNA | n | (mean) | (mean) | (s.e.m.) | (s.e.m.) |
| ZSCAN2 | 1x | 3 | 37.93 | 0.73 | 7.07 | 0.04 |
| sgRNA1 | | | | | | |
| OT2 | | | | | | |
| FANCF | 1 | 3 | 29.96 | 1.04 | 3.42 | 0.04 |
| sgRNA2 | | | | | | |
| OT1 | | | | | | |
| VEGFA | 8 | 3 | 13.11 | 1.02 | 1.57 | 0.08 |
| sgRNA2 | | | | | | |
| OT17 | | | | | | |
| CCR5-R30 | 3 | 3 | 11.34 | 0.57 | 6.81 | 0.28 |
| OT-CCR2 | | | | | | |
| ZSCAN2 | 3 | 3 | 8.95 | 0.82 | 2.07 | 0.04 |
| sgRNA1 | | | | | | |
| OT1 | | | | | | |
| VEGFA | 8 | 3 | 7.50 | 1.00 | 2.60 | 0.21 |
| sgRNA2 | | | | | | |
| OT2 | | | | | | |
| HBB-R01 | 2 | 3 | 7.48 | 0.89 | 1.74 | 0.18 |
| OT-HBD | | | | | | |
| VEGFA | 1 | 3 | 6.75 | 1.48 | 1.68 | 0.07 |
| sgRNA3 | | | | | | |
| OT4 | | | | | | |
| VEGFA | 2 | 3 | 6.72 | 0.93 | 0.97 | 0.10 |
| sgRNA1 | | | | | | |
| OT1 | | | | | | |
| HBB-R03 | 4 | 3 | 6.55 | 0.89 | 2.01 | 0.12 |
| OT-HBD | | | | | | |
| VEGFA | 8 | 3 | 4.99 | 0.51 | 1.37 | 0.14 |
| sgRNA1 | | | | | | |
| OT4 | | | | | | |
| VEGFA | 8 | 3 | 4.57 | 0.66 | 1.49 | 0.19 |
| sgRNA1 | | | | | | |
| OT6 | | | | | | |
| VEGFA | 1 | 3 | 4.32 | 1.05 | 0.92 | 0.10 |
| sgRNA2 | | | | | | |
| OT1 | | | | | | |
| VEGFA | 2 | 3 | 4.26 | 1.04 | 0.43 | 0.04 |
| sgRNA3 | | | | | | |
| OT2 | | | | | | |
| VEGFA | 5 | 3 | 2.13 | 1.33 | 1.13 | 0.50 |
| sgRNA3 | | | | | | |
| OT18 | | | | | | |
| VEGFA | 7 | 3 | 40.60 | 0.31 | 16.94 | 0.08 |
| sgRNA1 | | | | | | |
| OT11 | | | | | | |
| HBB-G10 | 7 | 3 | 3.74 | 0.47 | 1.49 | 0.08 |
| OT1 | | | | | | |
| VEGFA | 5 | 3 | 1.55 | 0.72 | 0.55 | 0.13 |
| sgRNA2 | | | | | | |
| OT19 | | | | | | |
| HBB-R04 | 4 | 3 | 1.16 | 0.77 | 0.29 | 0.09 |
| OT-HBD | | | | | | |

TABLE 2
dRNAs alone do not promote editing at sgRNA target sites. Difference
between indel frequencies at on- and off-target (OT) sites for
the best dRNA compared to a negative control at 12 different on/off-
target pairs (Δ). p: p-value, based on two-sided Student's t-test.
p_{adj}: Bonferroni-adjusted p-value. n = 3 biological replicates
at on- and off-target sites, except for VEGFA sgRNA3 OT2 (n =
9) and VEGFA sgRNA3 OT18 (n = 3 at on-target, n = 2
at off-target due to failed sequencing reactions).

| | Δ | p | p_{adj} | Δ | p | p_{adj} |
|---|---|---|---|---|---|---|
| Site | (On) | (On) | (On) | (OT) | (OT) | (OT) |
| FANCF | −0.004 | 0.835 | 1 | −0.002 | 0.910 | 1 |
| sgRNA2 | | | | | | |
| OT1 | | | | | | |
| HBB R03 | −0.014 | 0.845 | 1 | −0.009 | 0.761 | 1 |
| OT-HBD | | | | | | |
| VEGFA | 6.07E−04 | 0.403 | 1 | 6.61E−04 | 0.094 | 1 |
| sgRNA1 | | | | | | |
| OT1 | | | | | | |
| VEGFA | −1.95E−04 | 0.524 | 1 | 0.025 | 0.209 | 1 |
| sgRNA1 | | | | | | |
| OT6 | | | | | | |
| VEGFA | −0.045 | 0.912 | 1 | 0.083 | 0.124 | 1 |
| sgRNA2 | | | | | | |
| OT1 | | | | | | |
| VEGFA | −0.018 | 0.750 | 1 | −9.37E−04 | 0.683 | 1 |
| sgRNA2 | | | | | | |
| OT2 | | | | | | |
| VEGFA | 0.015 | 0.306 | 1 | 0.006 | 0.218 | 1 |
| sgRNA2 | | | | | | |
| OT17 | | | | | | |
| VEGFA | −0.007 | 0.907 | 1 | 0 | 1 | 1 |
| sgRNA3 | | | | | | |
| OT4 | | | | | | |
| VEGFA | −3.27E−04 | 0.513 | 1 | 0.002 | 0.319 | 1 |
| sgRNA3 | | | | | | |
| OT18 | | | | | | |
| ZSCAN2 | −3.87E−04 | 0.789 | 1 | 0 | 1 | 1 |
| sgRNA1 | | | | | | |
| OT1 | | | | | | |
| ZSCAN2 | 3.15E−05 | 0.479 | 1 | 0.001 | 0.092 | 1 |
| sgRNA1 | | | | | | |
| OT2 | | | | | | |
| VEGFA | 0.040 | 0.406 | 1 | 0.080 | 0.050 | 1 |
| sgRNA3 | | | | | | |
| OT2 | | | | | | |

TABLE 3
dRNAs alone do not promote editing at predicted dRNA target sites. Difference
between indel frequencies at on- and off-target (OT) sites for the best dRNA
compared to a negative control at 12 different on/off-target pairs (Δ).
Predicted indel locations (pred) are the location of expected indels if the dRNA
were a full length sgRNA. p: p-value, based on two-sided Student's t-test.
p_{adj}: Bonferroni-adjusted p-value. n = 3 biological replicates at on-
and off-target sites, except for VEGFA sgRNA3 OT2 (n = 9) VEGFA sgRNA2
OT17 (n = 3 at on-target, n = 2 at off-target due to failed sequencing
reactions), and VEGFA sgRNA3 OT18 (n = 3 at on-target, n = 2 at
off-target due to failed sequencing reactions).

| | Δ | p | p_{adj} | Δ | p | p_{adj} |
|---|---|---|---|---|---|---|
| Site | (On_{pred}) | (On_{pred}) | (On_{pred}) | (OT_{pred}) | (OT_{pred}) | (OT_{pred}) |
| FANCF | −0.004 | 0.835 | 1 | −0.002 | 0.910 | 1 |
| sgRNA2 | | | | | | |
| OT1 | | | | | | |
| HBB R03 | −0.056 | 0.699 | 1 | −0.006 | 0.828 | 1 |
| OT-HBD | | | | | | |
| VEGFA | −0.004 | 0.730 | 1 | 0.001 | 0.312 | 1 |
| sgRNA1 | | | | | | |
| OT1 | | | | | | |
| VEGFA | 7.78E−04 | 0.278 | 1 | 0.027 | 0.204 | 1 |
| sgRNA1 | | | | | | |
| OT6 | | | | | | |
| VEGFA | 0.316 | 0.220 | 1 | 0.083 | 0.124 | 1 |
| sgRNA2 | | | | | | |
| OT1 | | | | | | |
| VEGFA | 0.028 | 0.253 | 1 | 0.001 | 0.302 | 1 |
| sgRNA2 | | | | | | |
| OT2 | | | | | | |
| VEGFA | 0.092 | 0.115 | 1 | 0.074 | 0.260 | 1 |
| sgRNA2 | | | | | | |
| OT17 | | | | | | |
| VEGFA | −0.015 | 0.908 | 1 | 0 | 1 | 1 |
| sgRNA3 | | | | | | |
| OT4 | | | | | | |
| VEGFA | 6.55E−05 | 0.471 | 1 | −0.013 | 0.622 | 1 |
| sgRNA3 | | | | | | |
| OT18 | | | | | | |
| ZSCAN2 | 0.002 | 0.089 | 1 | −7.37E−04 | 0.539 | 1 |
| sgRNA1 | | | | | | |
| OT1 | | | | | | |
| ZSCAN2 | 3.15E−05 | 0.479 | 1 | 0.001 | 0.092 | 1 |
| sgRNA1 | | | | | | |
| OT2 | | | | | | |
| VEGFA | 0.061 | 0.094 | 1 | 0.073 | 0.071 | 1 |
| sgRNA3 | | | | | | |
| OT2 | | | | | | |

An important application of Cas9 is editing genes containing pathogenic mutations. For example, Cas9 has been used to target the β-globin locus (HBB), with the goal of curing Sickle Cell Disease. However, the δ-globin locus (HBD) is a common off-target for sgRNAs targeting HBB, and cleavage of both on- and off-target sites can result in large chromosomal deletions at the globin locus (see Cradick, T. J., et al., CRISPR/Cas9 systems targeting β-globin and CCR5 genes have substantial off-target activity. Nucleic Acids Res 41, 9584-9592 (2013)). In HEK-293T cells, dOTS decreased off-target editing at HBD from 1.08% (s.e.m.=0.22%, n=3) to 0.15% (s.e.m.=0.03, n=3). In Elf1 cells, dOTS decreased off-target editing at HBD from 20.72% (s.e.m.=2.75, n=3) to 1.20% (s.e.m.=0.18, n=3), increasing the specificity ratio from 1.33 to 13.72. Thus, dOTS can control unwanted editing at clinically relevant loci.

Initial attempts did not find effective dRNAs for four off-target sites. In two cases, dRNAs strongly reduced off-target editing but also decreased on-target editing by greater than two-fold (FIG. 1C). In two other cases, the tested dRNAs were not effective in decreasing off-target editing (FIG. 1C). Without being bound to any particular theory, it is postulated that these ineffective dRNAs are either unstable, form unfavorable secondary structures, or have insufficient affinity for the off-target site relative to their cognate sgRNAs. However, at the majority of off-targets one or more effective dRNAs were identified that enhanced specificity without sacrificing on-target editing, making dOTS an effective approach for off-target suppression. Only in a small minority of cases, additional optimization may be needed to ensure reduction in off-target editing while minimizing on-target interference.

Mechanism of Off-Target Suppression by dRNAs

dOTS is based on the theory that Cas9•dRNA complexes with perfect complementarity to an off-target site can directly outcompete active, imperfectly complementary Cas9•sgRNA complexes for binding. To test this Cas9 self-competition mechanism, in vitro cleavage assays were performed with linear DNA substrates and purified Cas9 ribonucleoprotein complexes (RNPs) containing either FANCF sgRNA2 or dRNA1. Incubation of a substrate containing the FANCF OT1 site with a mixture of the Cas9•dRNA1 and Cas9•sgRNA2 complexes led to a robust reduction in cleavage compared to administration of the Cas9•sgRNA2 complex alone (not shown). Consistent with the proposed self-competition mechanism, preincubation of the substrate with the Cas9•sgRNA2 complex for 10 minutes followed by addition of the Cas9•dRNA1 complex eliminated the reduction in cleavage (not shown). Thus, Cas9•dRNA complexes can directly shield off-target loci from Cas9•sgRNA cleavage.

At low concentrations of Cas9•sgRNA2, Cas9•dRNA1 modestly reduced cleavage of the on-target FANCF substrate site in vitro (not shown), despite this dRNA not affecting on-target editing efficiency in cells (FIGS. 1B, 1D, and 1E). One possible explanation for this disparity is that, in cells, Cas9•dRNA1-mediated protection of the on-target locus decreases the rate of indel formation but editing reaches the same maximum as in cells without dRNA1 by the time of measurement. Another explanation is that cellular factors prevent Cas9•dRNA1, which should have modest affinity for the on-target site, from providing appreciable protection from cleavage by Cas9•sgRNA2. Thus, rates of indel formation were measured at FANCF sgRNA2 OT1 and the on-target site in cells using a chemically-inducible Cas9 (ciCas9) variant. The activity of ciCas9 is repressed by an intramolecular autoinhibitory switch. Addition of a small molecule, A-1155463 (A115), disrupts autoinhibition and rapidly activates ciCas9, enabling precise studies of editing kinetics.

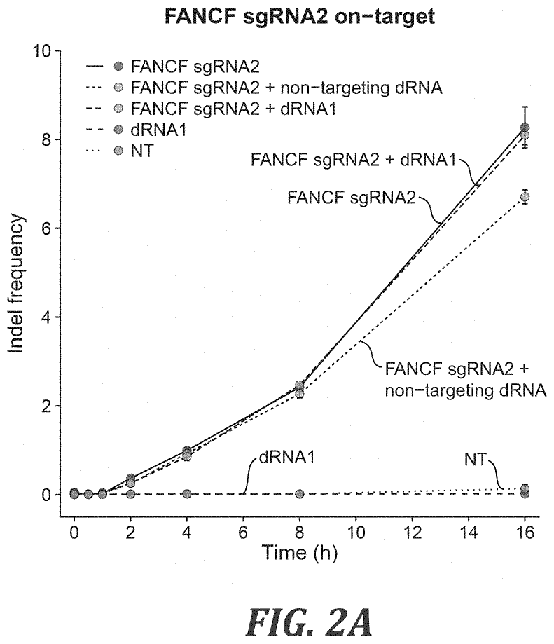

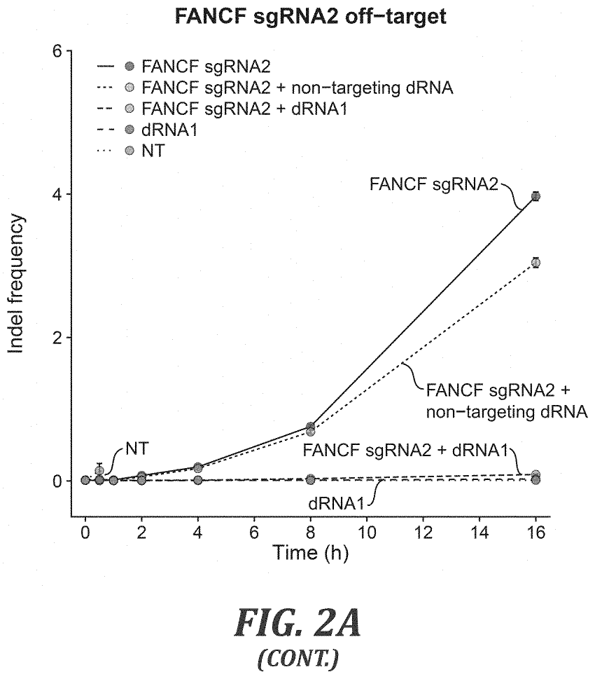

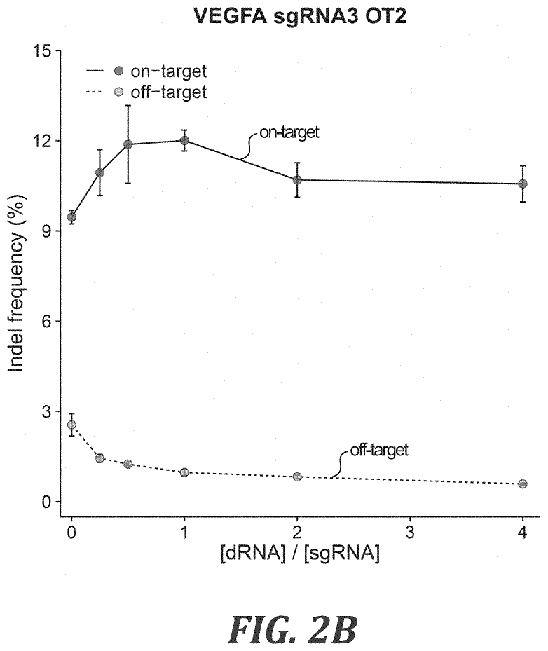

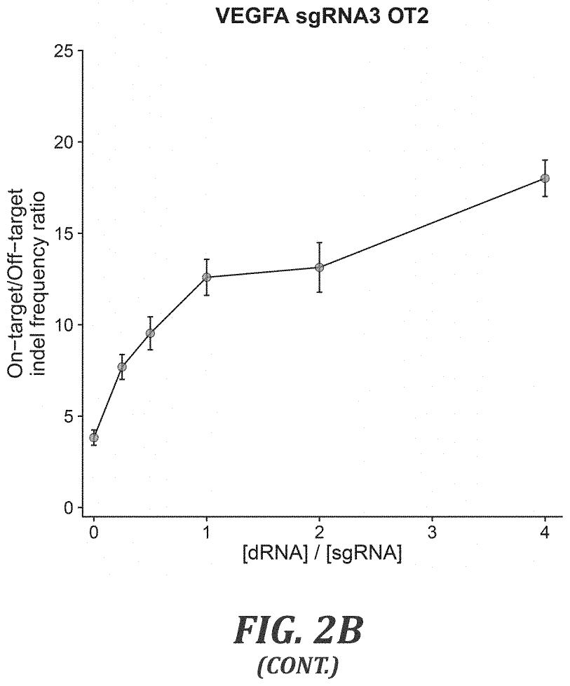

As expected, activation of ciCas9 with A115 led to the rapid appearance of indels at the FANCF sgRNA2 on- and off-target sites in the absence of dRNA1. Inclusion of a plasmid encoding dRNA1 effectively eliminated ciCas9-mediated editing at the off-target site but had no measurable impact on the kinetics of on-target editing (FIG. 2A). These results suggest that dRNAs with imperfect complementarity to an on-target site can bind to and protect that site in cell-free systems, but not in cells. The most likely explanation for this difference is that, in cells, DNA is subject to a variety of active processes that influence Cas9. For example, the degree of complementarity between a guide and its target affects the ability of polymerases to displace dCas9 from DNA, suggesting that polymerases may limit the ability of imperfectly complementary Cas9•dRNA complexes to shield on-target sites.

The proposed Cas9 self-competition mechanism predicts that the level of off-target shielding provided by moderately effective dRNAs can be improved by manipulating the ratio of Cas9•dRNA to Cas9•sgRNA in cells. While an initial 1:1 plasmid ratio was effective for all 15 successful dRNAs, increasing the amount of dRNA relative to sgRNA further decreased off-target editing and improved the specificity ratio at each of the four sgRNA/dRNA pairs that were tested (FIG. 2B). Similar results were observed in additional titration assays with sgRNA/dRNA pairs directed to other sites, including FANCF sgRNA2 and dRNA1, HBB R03 and dRNA4; and ZSCAN2 sgRNA1 and dRNA3 (not shown). For one pair, higher dRNA:sgRNA ratios also decreased on-target editing. Thus, a trade-off between maintaining on-target editing and decreasing off-target editing exists for some sgRNA/dRNA pairs. Here, the dRNA/sgRNA ratio can be tuned based on whether preserving on-target editing or suppression of a particular off-target is desired.

dOTS Improves Other Approaches to Increase Cas9 Specificity

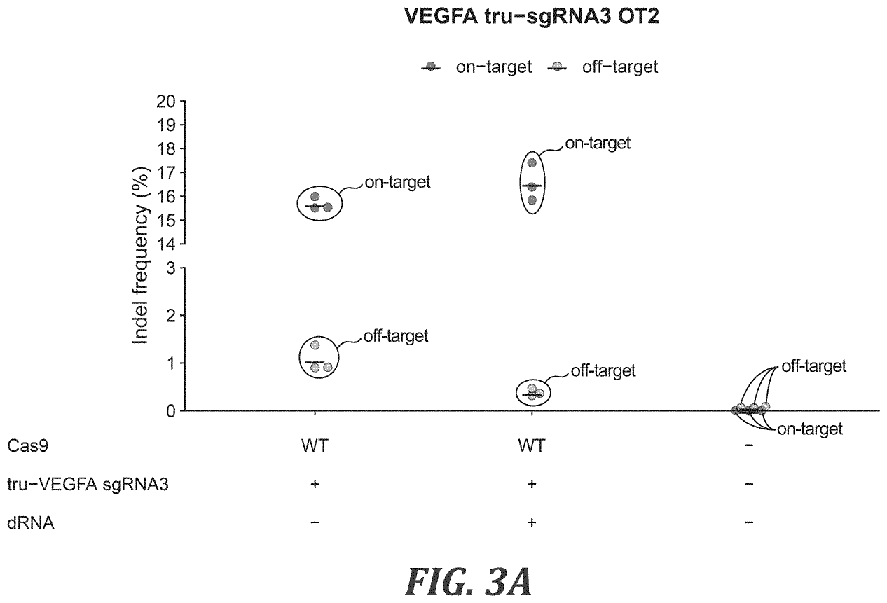

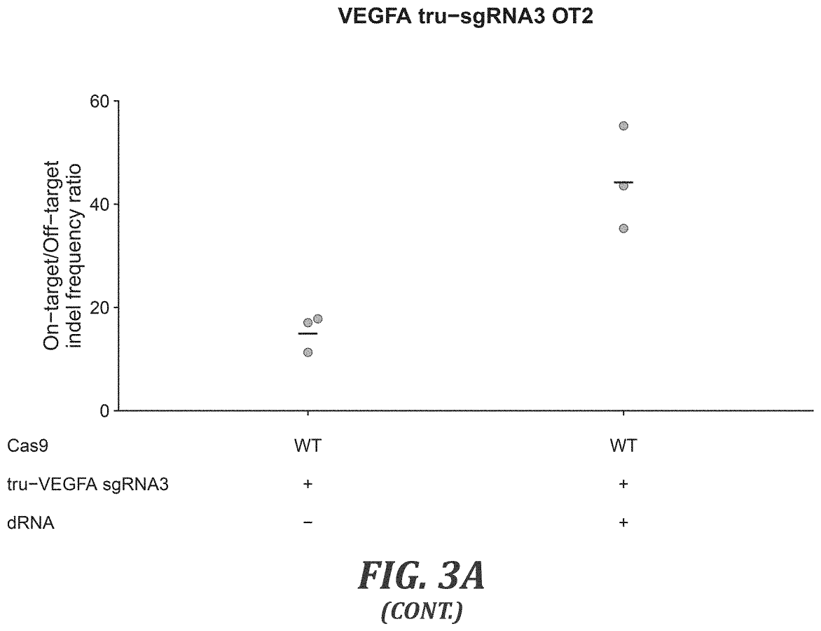

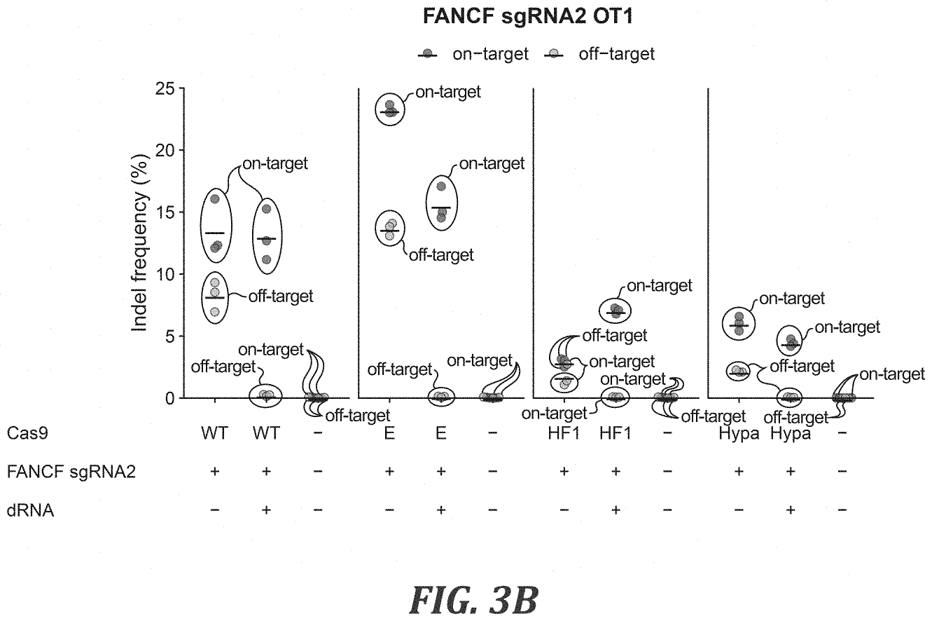

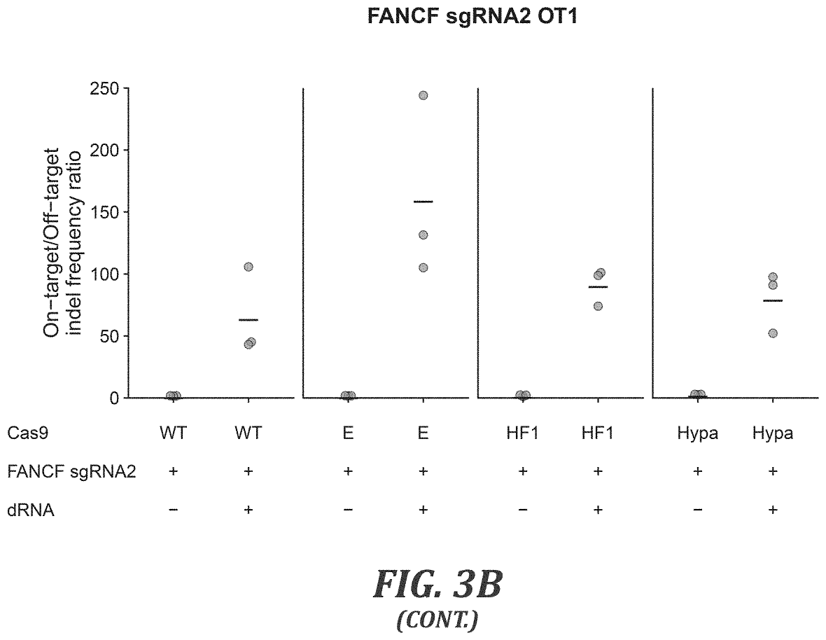

Previously known strategies to improve Cas9 specificity fail to completely suppress off-target editing and often reduce on-target efficacy. Thus, the inventor addressed the question of whether such strategies could be enhanced with dOTS. One prior approach uses truncated sgRNAs (tru-sgRNAs) with 17-19 base target sequences to increase on-target specificity at some loci. For example, truncation of the VEGFA sgRNA3 target sequence (VEGFA tru-sgRNA3) decreases editing at some off-target sites, but editing at OT2 remains. dOTS suppressed editing at this refractory off-target site without affecting on-target editing (FIG. 3A), demonstrating that it is compatible with tru-sgRNAs.

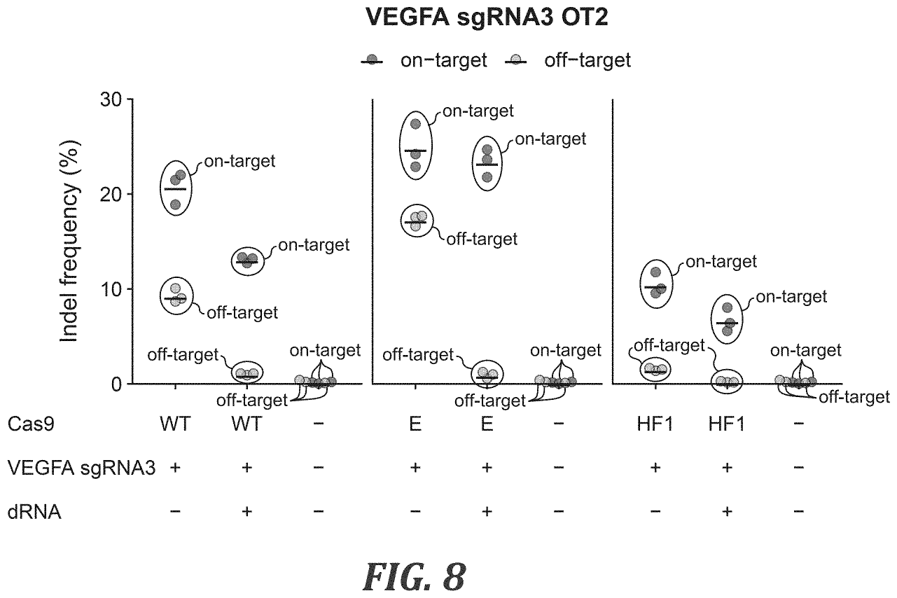

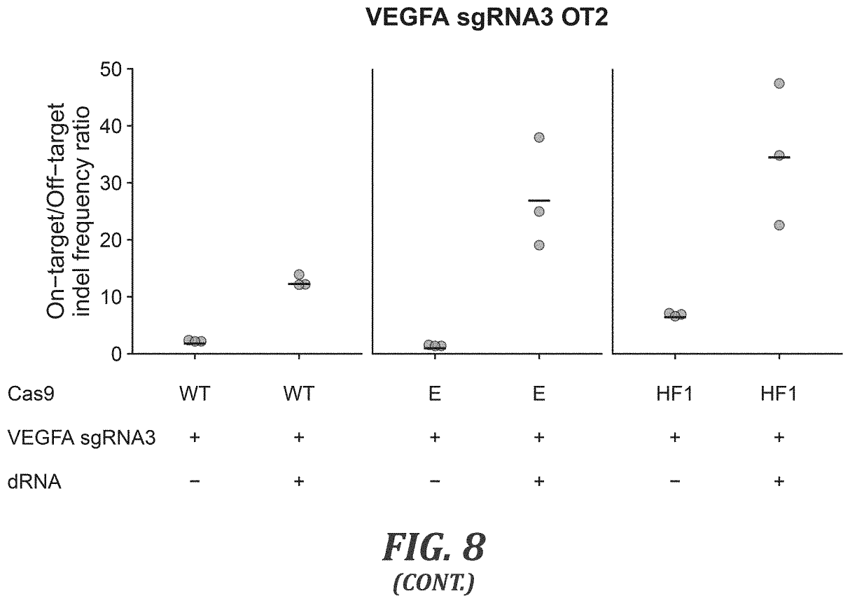

More recently, rational engineering of SpCas9 has produced high-specificity variants like eSpCas9(1.1), SpCas9-HF1, and HypaCas9. While these variants generally improve on-target specificity, they do not suppress unwanted editing at all off-target sites for all sgRNAs. For example, a recent evaluation of these three high-specificity variants revealed off-target editing by all three variants for four of the six sgRNAs tested. In another example, FANCF sgRNA2 OT1 is still edited at high frequencies by all three high-specificity variants (FIG. 3B). Co-transfection of FANCF sgRNA2 with an effective dRNA reduced off-target editing to levels indistinguishable from non-transfected controls for all high-specificity Cas9 variants (P>0.05, one-sided t-test, n=3), dramatically increasing specificity ratios (FIG. 3B). dRNAs also effectively suppressed off-target editing by eSpCas9(1.1) and SpCas9-HF1 at a refractory VEGFA sgRNA3 off-target (FIG. 8). High-specificity Cas9 variants are known to exhibit decreased on-target activity, which is sensitive to delivery method and other factors. Indeed, in some cases, a decrease was observed in on-target editing when high-specificity Cas9 variants and dOTS are combined. However, this reduction in on target editing is generally less pronounced than the efficiency loss observed comparing HypCas9 or SpCas9-HF1 to wild-type in the absence of dOTS. The reduction in on-target editing is also markedly less than the degree of suppression achieved by dOTS at the off-target site. Thus, dOTS can be combined with many other methods for improving Cas9 specificity.

dOTS can be Multiplexed to Suppress Multiple Off-Targets

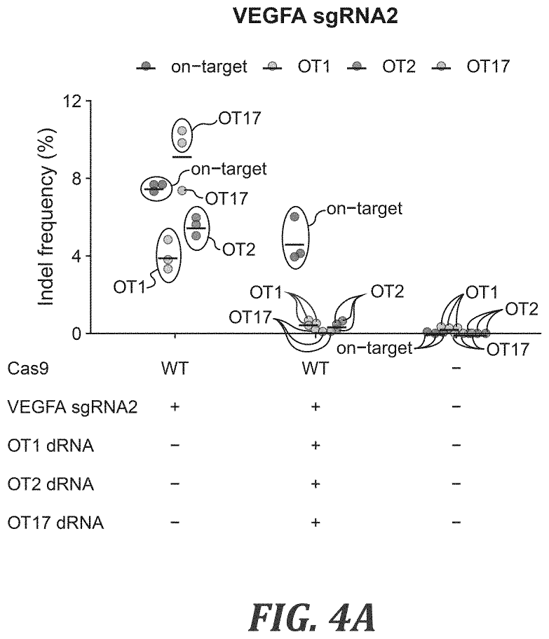

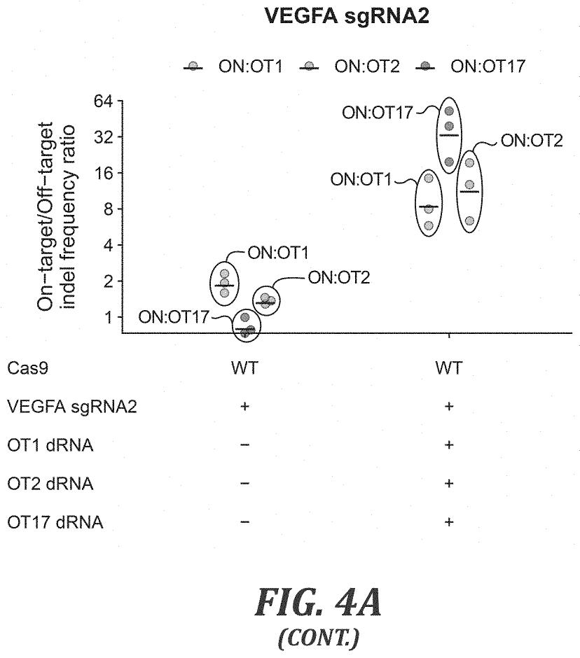

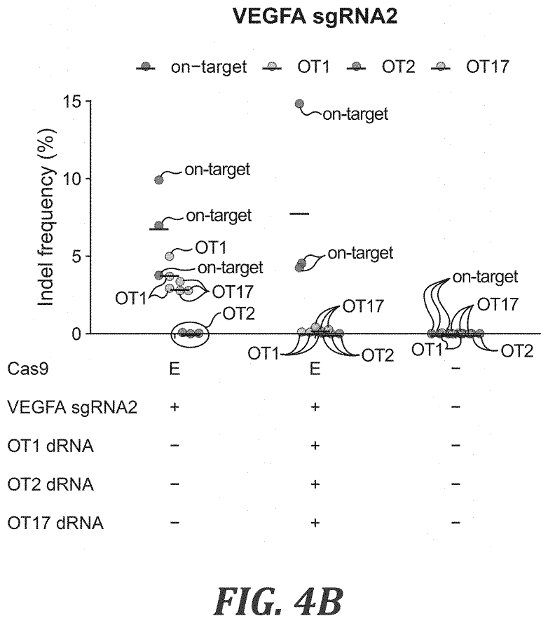

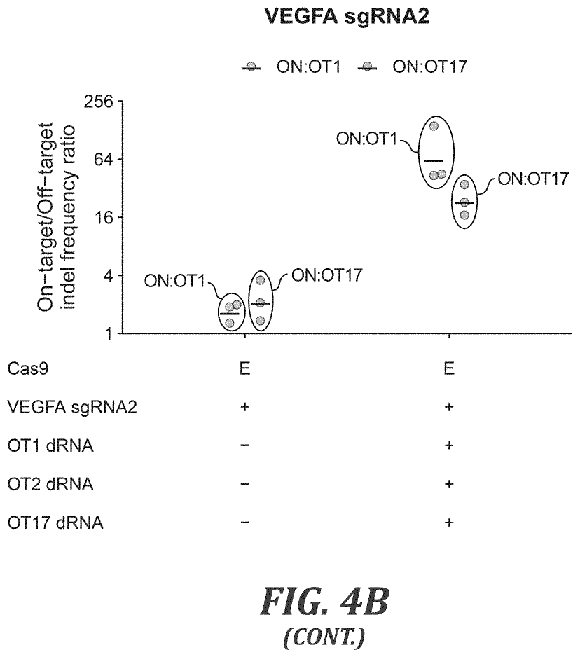

Considering that many sgRNAs induce off-target editing at numerous sites, the question of whether dOTS could be multiplexed was examined. Three off-target sites were selected for VEGFA sgRNA2 with individually effective dRNAs (FIG. 1C). HEK-293T cells were transfected with VEGFA sgRNA2 and the dRNAs individually, in duplex, or in triplex. Even when all three dRNAs were combined, editing at each off-target site was suppressed by its cognate dRNA with only small losses in on-target editing (FIG. 4A) shows representative trends based on assays with VEGFA sgRNA2 and dRNAs targeting one of three VEGFA sgRNA2 off-targets (OT1 dRNA1, OT2 dRNA8, OT17 dRNA8) with both WT Cas9 or eSpCas9. Multiplex dOTS was also effective for two other sgRNAs (i.e., VEGFA sgRNA3 combined with dRNAs targeting OT2, OT4, and OT18, ZSCAN2 sgRNA1 combined with dRNAs targeting OT1 and OT2) (not shown). An additional assay combining two distinct sgRNAs and corresponding dRNAs, i.e., FANCF sgRNA2 combined with OT1 and ZSCAN2 sgRNA1 combined with OT2 in the same assay) demonstrated that the approach could even suppress the off-targets of the two distinct sgRNAs simultaneously (not shown). Notably, each dRNA only impacted editing at its cognate off-target site, without increasing or decreasing the editing at the other off-target sites of the sgRNA.

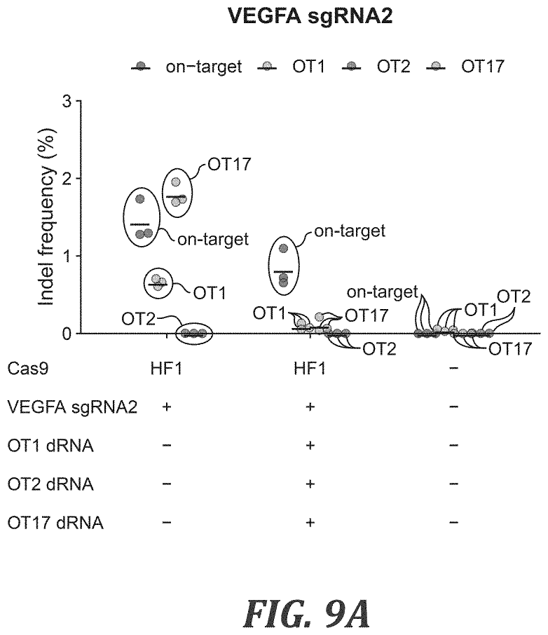

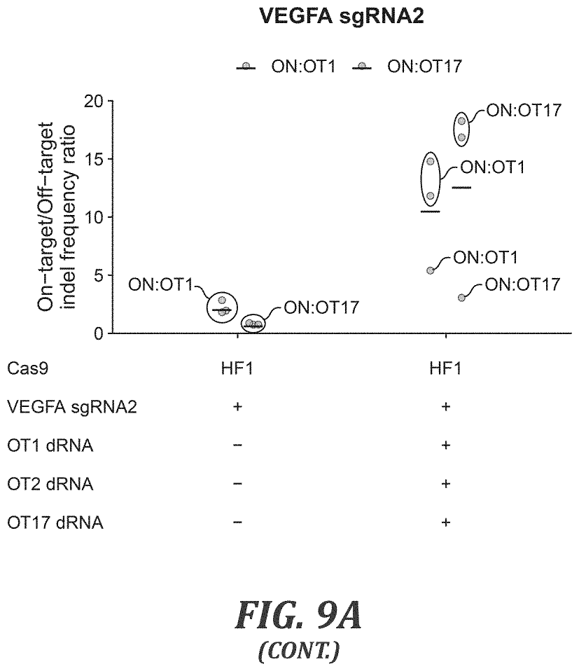

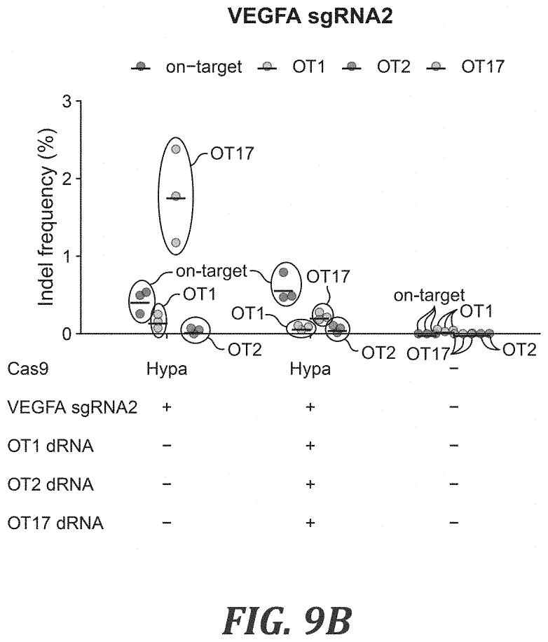

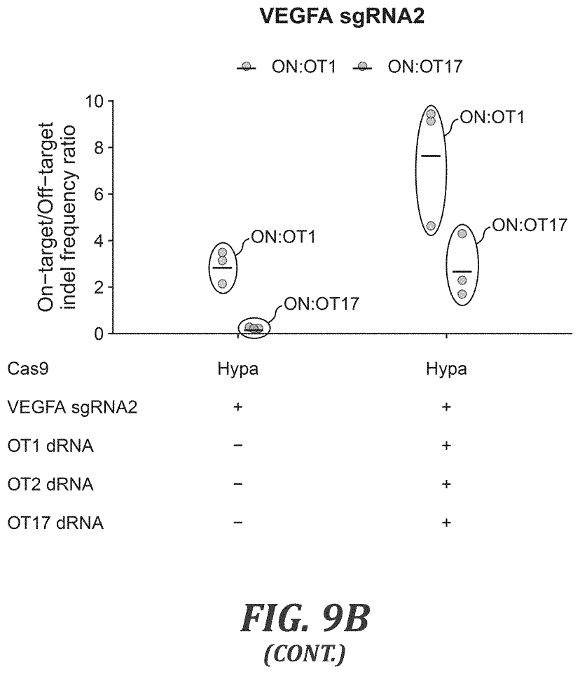

Like wild type Cas9, high-specificity Cas9 variants can cause editing at multiple off-target sites. For example, eSpCas9 reportedly drives appreciable editing with VEGFA sgRNA2 at three different off-target sites. Off-target editing was observed at two of these sites, and it was found that dRNAs could simultaneously decrease off-target editing at both sites without perturbing on-target editing (FIG. 4B). Furthermore, multiplexed dOTS suppressed editing driven by SpCas9-HF1 and HypaCas9 at these off-target sites (FIGS. 9A and 9B). Thus, in the context of both wild type and variant Cas9, dRNAs can be combined to suppress multiple off-targets simultaneously.

dRNAs Enable Scarless HDR-Mediated Genome Editing

When mutations introduced by HDR do not substantially disrupt the target sequence or PAM, as is generally the case for single nucleotide variants, Cas9 can continue to cleave the target site after repair. Continued cleavage introduces indels, substantially decreasing the frequency of loci containing the desired sequence. For example, quantification of editing outcomes at PSEN1 revealed that up to 95% of HDR-corrected templates showed secondary indels due to recutting. If a protein-coding region is being edited, synonymous blocking mutations that disrupt the sgRNA target sequence, PAM, or both are generally included in the repair template. Unfortunately, synonymous blocking mutations may alter protein expression or interfere with mRNA splicing. Furthermore, predicting functionally neutral blocking mutations in non-coding regions is extremely challenging. Base editing can in some cases make single base changes, yet its use is hindered by unwanted bystander editing within the editing window, off-target editing of RNA, and an inability to install transversion mutations or targeted insertions and deletions. Thus, “scarless editing”, the ability to efficiently introduce single nucleotide variants and other small changes into the genome via HDR without blocking mutations or unwanted indels, would be of tremendous utility.

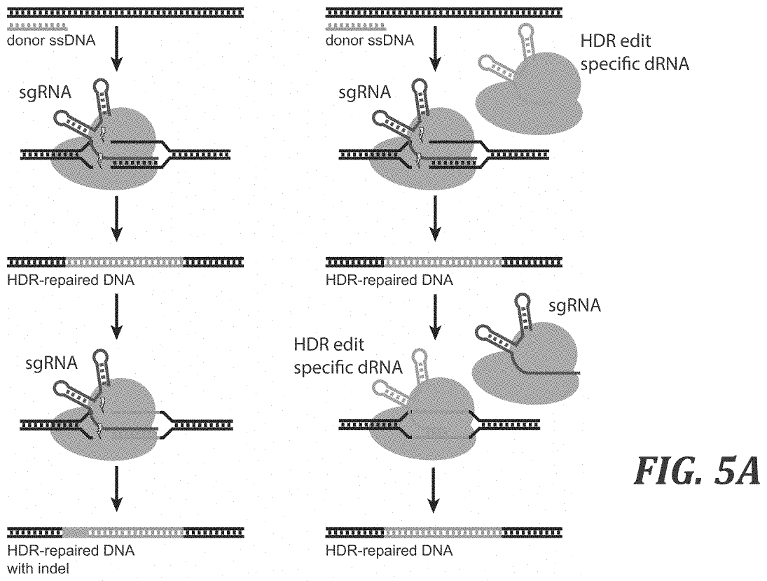

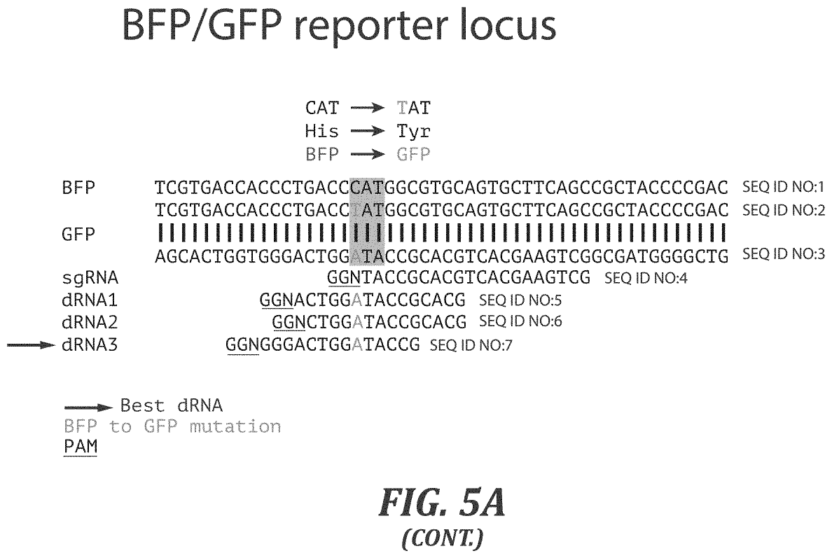

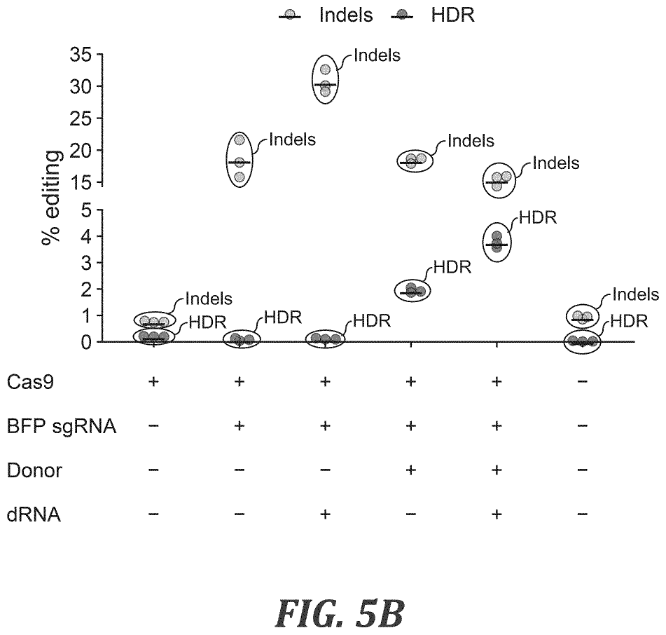

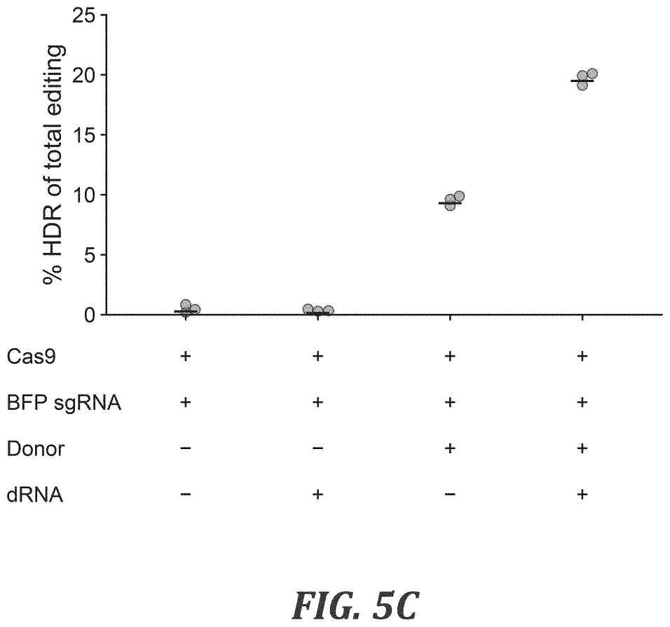

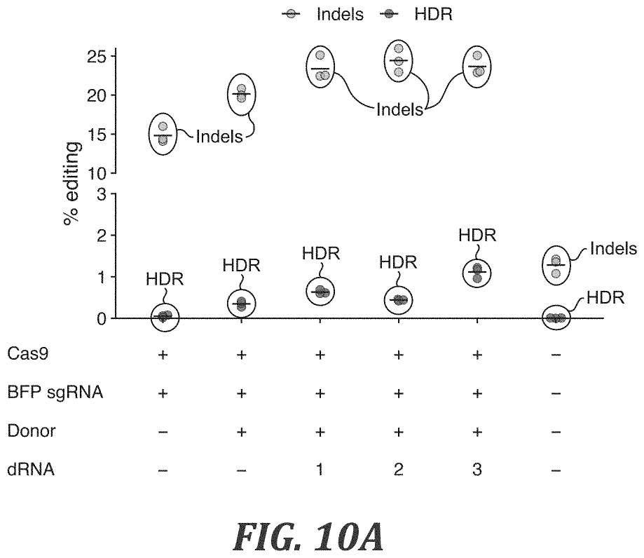

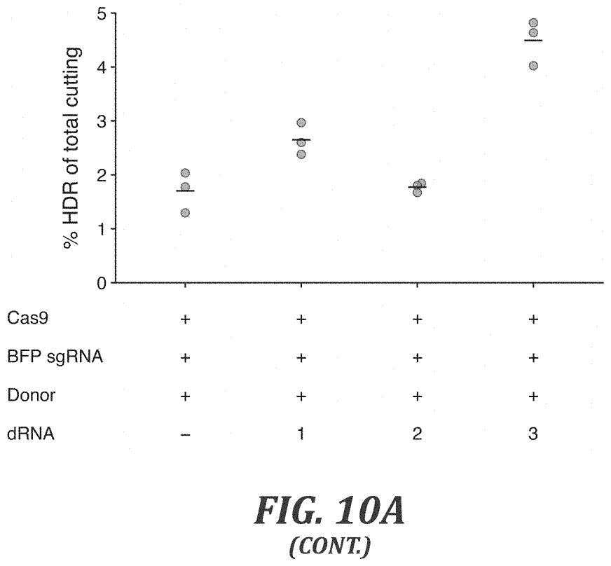

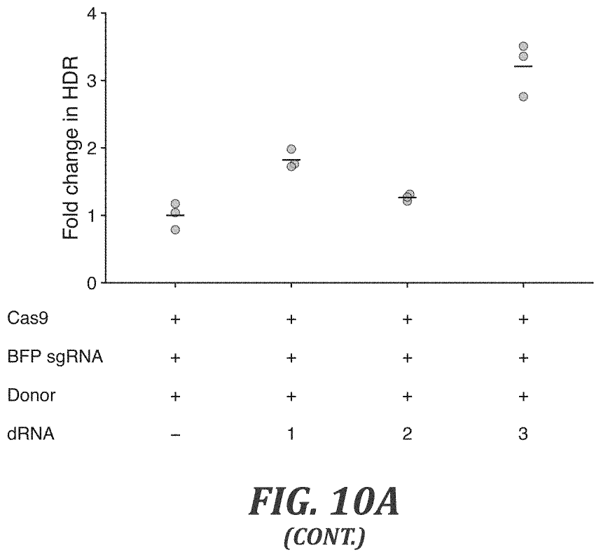

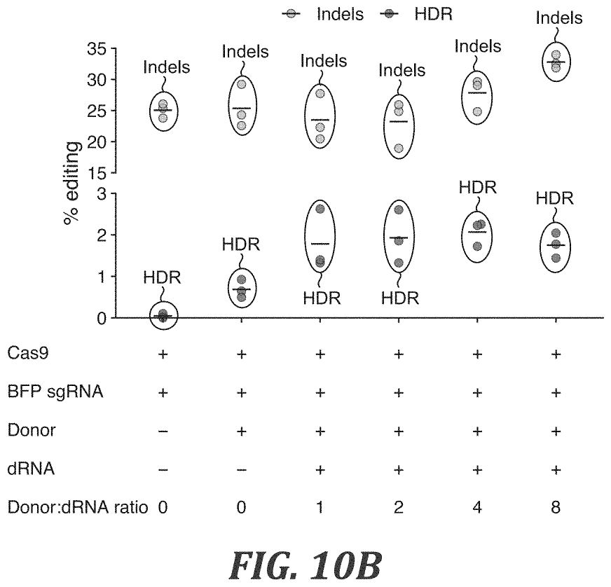

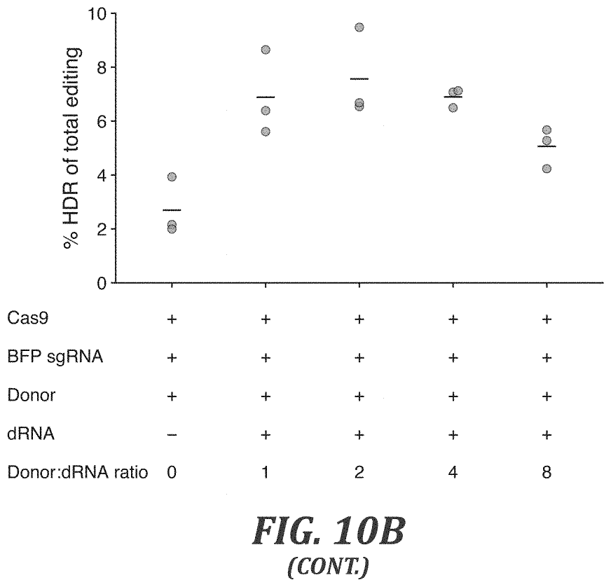

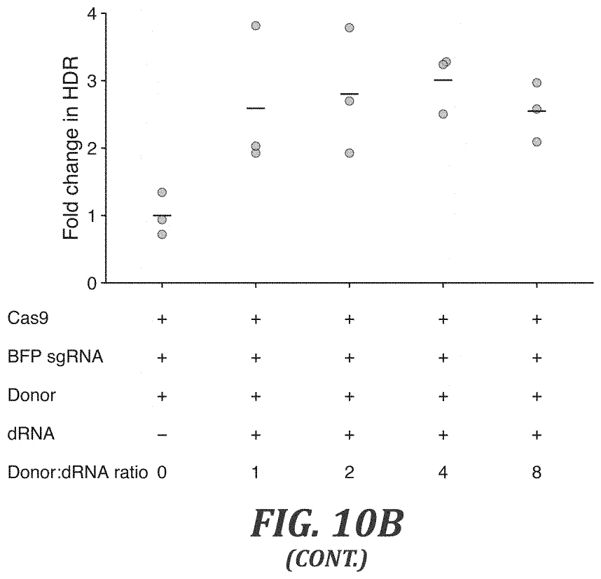

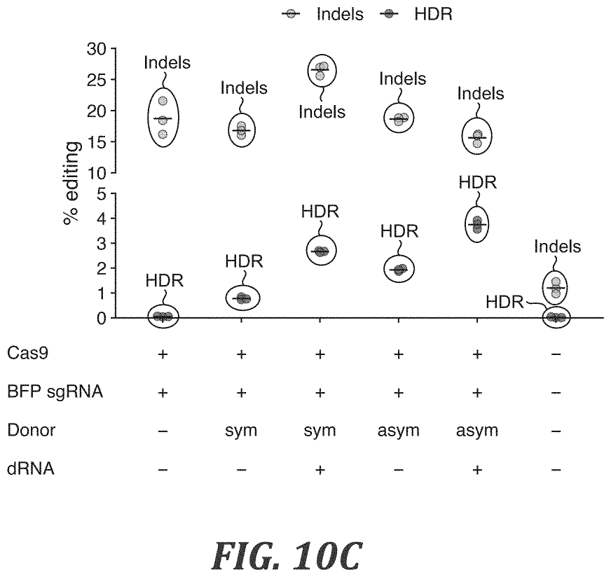

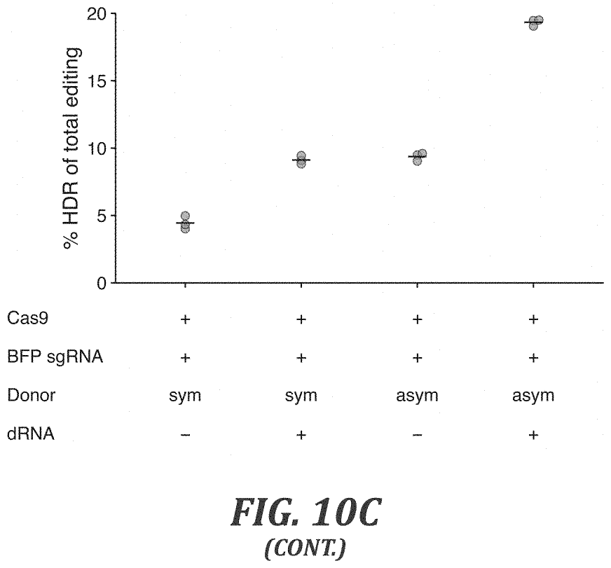

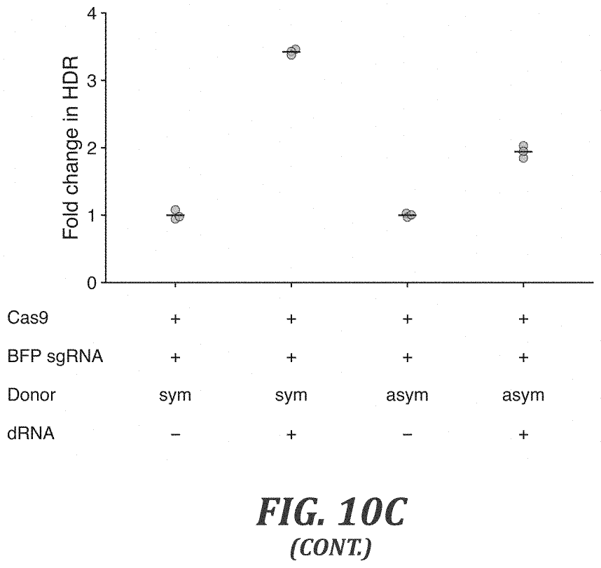

The inventors predicted that dRNAs directed at a desired, HDR-corrected sequence could shield repaired sites from recutting, an approach referred to as dRNA-mediated Re-Cutting Suppression (“dReCS”; FIG. 5A). The ability of dRNAs to improve the HDR-mediated conversion of BFP to GFP through substitution of a single amino acid was evaluated. Previously, several blocking mutations were used to prevent recutting, yet only a single nucleotide change is needed to alter the His in BFP (CAT) to the Tyr in GFP (TAT). A previously used sgRNA in which the permissive site within the PAM (i.e. N in NGG) for the BFP sgRNA corresponds to the mutated nucleotide was selected. Thus, this sgRNA possesses perfect complementarity to both the native and HDR-repaired locus, representing a worst-case scenario in which Cas9•sgRNA is expected to efficiently recut HDR-repaired sites. HEK-293T cells with stably integrated BFP were transfected with a single stranded oligodeoxynucleotide (ssODN) donor template containing the single nucleotide change, the sgRNA targeting BFP, and one of three dRNAs with perfect complementarity to the GFP but not BFP sequence. After four days, in the absence of dRNA, scarless HDR conversion to GFP was inefficient, with 1.94% of cells expressing GFP by flow cytometry. In the presence of the best dRNA, absolute HDR efficiency increased to 3.77% (FIGS. 5B and 10A-10C), corresponding to an increase in the percentage of all edited sites exhibiting scarless HDR from 9.53% (s.e.m.=0.40, n=3) to 19.72% (s.e.m.=0.52, n=3; FIG. 5C). Thus, dReCS can promote scarless HDR even when the sgRNA has perfect complementarity for the HDR corrected sequence.

### DISCUSSION

Here, a general approach is described for the targeted suppression of unwanted Cas9-mediated editing that relies on co-administration of dRNAs with complementarity to the suppressed site. The disclosed approach exploits the previously unappreciated phenomenon referred to herein as Cas9 self-competition: the ability of different Cas9•guide RNA complexes to compete for a limited number of genomic target sites. It is demonstrated here that catalytically inactive Cas9, in this case Cas9 bound to a dRNA, can protect sites from undesired cleavage by active Cas9•sgRNA complexes. One application of this approach, dRNA mediated off-target suppression (dOTS), reduced editing at 15 distinct off-target sites, in some cases below the limit of detection by high-throughput sequencing. Another application, dRNA recutting suppression (dReCS), facilitated the scarless introduction of a single base change that did not impact the PAM or target sequence. dReCS circumvents the need for blocking mutations, making it particularly useful for single nucleotide variants and small indels in non-coding regions of the genome where synonymous blocking mutations are not an option. In both cases, effective dRNAs can generally be rapidly identified with minimal screening. Moreover, dRNAs are effective in a variety of different cell lines and they can be combined to protect multiple off-target sites simultaneously.

dOTS and dReCS offer many advantages. However, in a minority of cases some additional optimization is required. In the initial design for a panel of targets, effective dRNAs for four of the 19 target/off-target pairs did not perform optimally. In some cases, additional dRNAs can be screened or the off-target member sequence can be further modified, but the sequence restrictions imposed by the SpCas9 NGG PAM mean that effective dRNAs may not always exist. One alternative is to improve poorly performing dRNAs by manipulating dRNA/sgRNA ratios. Another is to combine dRNAs with the recently described xCas9 or SpCas9-NG variants, which have a more permissive PAM that increases the number of candidate dRNAs (see, e.g., Hu, J. H., et al., Evolved Cas9 variants with broad PAM compatibility and high DNA specificity. Nature 556, 57-63 (2018), and Nishimasu, H., et al., Engineered CRISPR-Cas9 nuclease with expanded targeting space. Science 361, 1259-1262 (2018), each of which is incorporated by reference in its entirety). Another drawback is that some dRNAs decrease on-target editing, particularly when they are multiplexed to suppress several off-target sites simultaneously. Without being bound to a particular theory, it may be that these losses in on-target editing likely arise due to dilution of the plasmids or competition between sgRNAs and dRNAs to complex with Cas9. The first issue could be addressed by using a multiplex guide expression scheme (see, e.g., Kabadi, A. M., et al., Multiplex CRISPR/Cas9-based genome engineering from a single lentiviral vector. Nucleic Acids Res 42, e147-e147 (2014), and Gu, B. et al., Transcription-coupled changes in nuclear mobility of mammalian cis-regulatory elements. Science 359, 1050-1055 (2018), each of which is incorporated by reference in its entirety), and both could be addressed by delivering preformed ribonucleoprotein (RNP) mixtures (Kim, S., et al., Highly efficient RNA-guided genome editing in human cells via delivery of purified Cas9 ribonucleoproteins. Genome Res. 24, 1012-1019 (2014), incorporated herein by reference in its entirety). Finally, dRNAs could yield unwanted transcriptional off-target effects. However, transcriptional repression by Cas9 in the absence of a repressive domain is modest, and such effects would be transient unless both Cas9 and the dRNA were integrated into the genome.

Other approaches for minimizing off-target editing are also imperfect, as they reduce on-target efficiency, introduce new off-target sites, limit the number of potential target sites, or demand difficult Cas9 engineering. Moreover, many of these approaches are laborious to implement in experimental models where Cas9 or a variant thereof has already been stably integrated into the genome. Finally, these existing methods are generally incompatible with each other, meaning they cannot be used in concert to minimize limitations and improve performance. In contrast, dOTS and dReCS are comparatively easy to use, low-cost, and flexible. For example, dOTS could be used to address refractory off-targets of the popular engineered high-specificity Cas9 variants (see, e.g., Kleinstiver, B. P., et al., High-fidelity CRISPR-Cas9 nucleases with no detectable genome-wide off-target effects. Nature 529, 490-495 (2016); Slaymaker, I. M., et al., Rationally engineered Cas9 nucleases with improved specificity. Science 351, 84-88 (2016); Chen, J. S., et al., Enhanced proofreading governs CRISPR-Cas9 targeting accuracy. Nature 550, 407-410 (2017); Vakulskas, C. A., et al., A high-fidelity Cas9 mutant delivered as a ribonucleoprotein complex enables efficient gene editing in human hematopoietic stem and progenitor cells. Nature Medicine 24, 1216-1224 (2018); Lee, J. K., et al., Directed evolution of CRISPR-Cas9 to increase its specificity. Nature Communications 9, 1-10 (2018); Kulcsár, P. I., et al., Crossing enhanced and high fidelity SpCas9 nucleases to optimize specificity and cleavage. Genome Biology 18, 190 (2017); and Casini, A., et al., A highly specific SpCas9 variant is identified by in vivo screening in yeast. Nature Biotechnology 36, 265-271 (2018), each of which is incorporated herein by reference in its entirety). Here, it is demonstrated that dOTS can effectively suppress editing at four refractory off-target sites with three high-specificity Cas9 variants. Using dOTS to address these refractory off-targets is also far less laborious and time-intensive than further Cas9 engineering, as has been done previously. Additionally, dReCS is simpler and less time-consuming than CORRECT, a previous approach for scarless HDR editing that requires multiple rounds of HDR to introduce and subsequently remove blocking mutations. Because of their flexibility and technical simplicity, dOTS and dReCS can be readily integrated with existing protocols and experimental systems, enabling refinement of genome editing with minimal effort.

The flexibility of dOTS and dReCS means that they have applications beyond those demonstrated herein for proof of concept. For instance, dOTS can facilitate allele-specific editing, even when the two alleles cannot be distinguished by a Cas9•sgRNA complex alone. Based on the principle of Cas9 self-competition, electroporation of Cas9•dRNA RNPs to quench editing by the active Cas9•sgRNA RNP should allow fine tuning of editing efficiencies. Similarly, dOTS can be employed to modulate the editing rates in CRISPR lineage tracing. Finally, dOTS and dReCS are likely to be effective with other CRISPR enzymes, such as SaCas9 or Cpf1. Thus, dOTS and dReCS are easy-to-implement, effective and complementary methods for refining genome editing in both research and clinical applications.

### Methods

Expression Plasmids

All sgRNA and dRNA target sequences, except for VEGFA sgRNAs, were cloned into the gRNA_Cloning Vector according to the hCRISPR gRNA synthesis protocol published by Addgene.org (online at addgene.org/static/data/93/40/adf4a4fe-5e77-11e2-9c30-003048dd6500.pdf). gRNA_Cloning Vector (Addgene plasmid 41824), VEGFA site #1 (‘VEGFA sgRNA1’) (Addgene plasmid 47505), VEGFA site #2 (‘VEGFA sgRNA2’) (Addgene plasmid 47506) and VEGFA Site #3 (‘VEGFA sgRNA3’) (Addgene plasmid 47507) were gifts.

An N-terminal FLAG tag sequence was appended via Gibson Assembly Cloning (New England Biosciences) to a human codon optimized Cas9 (subcloned from hCas9; Addgene plasmid 41815) with a single C-terminal NLS expressed from a pcDNA3.3-TOPO vector. This was subsequently cloned into the pcDNA5/FRT/TO backbone (ThermoFisher). High-specificity variants of Cas9-eSpCas9(1.1) (Addgene plasmid 71814) and VP12 (′SpCas9-HF1′; Addgene plasmid 72247) were subcloned into pcDNA5/FRT/TO backbone (ThermoFisher). HypaCas9 (‘BPK4410’) (Addgene plasmid 101178).

The sequences of all plasmids, primers and other DNA constructs used in this work can be found in Supplementary Data Set.

Cell Culture

HEK-293T cells (293T/17, ATCC) were maintained in high-glucose DMEM supplemented with 10% fetal bovine serum (FBS, Life Technologies). U2OS cells (ATCC) were maintained in McCoy's 5A (modified) medium supplemented with 10% FBS (Life Technologies). hESC Elf1 iCas9 (Ferreccio, A., et al., Inducible CRISPR genome editing platform in naive human embryonic stem cells reveals JARID2 function in self-renewal. Cell Cycle 17, 535-549 (2018), incorporated herein by reference in its entirety) were plated into matrigel-coated 24-well plates and cultured in MEF-conditioned media supplemented with 2iL-I-F (GSK3i, MEKi, LIF, IGF, bFGF). All cell lines were regularly tested and confirmed free from mycoplasma contamination.

Genome Editing by Cas9

Unless otherwise specified, HEK-293T cells were plated in 24-well plates at 1.5×10⁵ cells/well. The day after plating, cells were transfected with Turbofectin 8.0 (Origene). For all dOTS experiments, 1.5 μL of Turbofectin 8.0 and 500 ng of plasmid DNA were transfected. For dRNA screening experiments, the plasmid DNA mixture contained 250 ng Cas9 (eSpCas9, Cas9-HF1, or HypaCas9), 125 ng sgRNA, and 125 ng dRNA. For wells without dRNA, the 125 ng of pMAX-GFP was substituted for the dRNA plasmid as a transfection control. For multiplex dOTS experiments, the plasmid DNA mixture contained 250 ng Cas9, 125 ng sgRNA, and 125 ng each of 1-3 dRNAs. A pMAX-GFP plasmid was used to increase total DNA transfected per well to 750 ng. U2OS cells were plated in 12-well plates at 7.5×10⁴ cells/well. The next day they were transfected with 3 μL of Turbofectin 8.0 and a total of 1 μg plasmid DNA (500 ng Cas9, 250 ng sgRNA, and 250 ng dRNA or pMAX-GFP plasmid). For titration experiments with all sgRNAs except VEGFA sgRNA3, HEK-293T cells were transfected with 1.5 μL of Turbofectin 8.0 and 500 ng of plasmid DNA. This DNA mixture contained 250 ng Cas9. The remaining 250 ng of DNA was divided between sgRNA and dRNA at varying ratios such that the total DNA was kept constant across experiments (1:1, 125 ng each sgRNA and dRNA; 1:2, 83.3 ng sgRNA and 166.7 ng dRNA; 1:4, 50 ng sgRNA and 200 ng dRNA; 2:1, 166.7 ng sgRNA and 83.3 ng dRNA; and 4:1, 200 ng sgRNA and 50 ng dRNA). For wells without dRNA, 125 ng of pMAX-GFP plasmid was substituted for the dRNA plasmid as a transfection control. For titration experiments with VEGFA sgRNA3, HEK-293T cells were transfected as above, but the DNA mixture contained 166.5 ng Cas9, and the various sgRNA:dRNA ratios were as follows (1:1, 166.5 ng each sgRNA and dRNA; 1:2, 111 ng sgRNA and 222 dRNA; 1:4, 66.6 ng sgRNA and 266.4 ng dRNA; 2:1, 222 ng sgRNA and 111 ng dRNA; 4:1, 266.4 ng sgRNA and 66.4 ng dRNA). For wells without dRNA, 166.5 ng of pMAX-GFP plasmid was substituted for the dRNA plasmid as a transfection control.

To harvest HEK-293T and U2OS cells for dOTS experiments, 24 hours after transfection each well of a 24-well plate was resuspended by thorough pipetting with 400 μL ice-cold DPBS. Resuspended cells were then spun at 1,500×g for 10 min at 4° C. DPBS was then aspirated and cell pellets were stored at −80° C. until genomic DNA isolation. For extended timepoint experiments, the same protocol was followed, except cells were passaged into a new 24 well plate after 24 hours after transfection and then subsequently harvested 48 hours after passaging.

Two days prior to plating, hESC Elf1 iCas9 cells were treated with 2 μg/ml doxycycline to induce Cas9 expression. At day 0, 2.5×10⁴ cells were plated into each well of a 24-well plate with addition of fresh doxycycline (2 μg/ml) and 10 μM Rock inhibitor to promote cell survival. After 24 hours, cells were transfected with 3 μL of Genejuice (EMD Millipore) and 1 μg plasmid DNA. This plasmid DNA mixture contained 500 ng sgRNA and 500 ng dRNA. For wells without dRNA, 500 ng of pMAX-GFP was substituted as a transfection control.

For Elf1 cells, 48 hours after transfection, each well of a 24-well plate was rinsed once with 0.5 mL DPBS and incubated for 5 min with trypsin to detach cells. 5 mL hESC media was added and the cells were spun down at 290×g for 3 min. The pellet was then washed with 1 mL DPBS, spun again at 290×g for 3 min then flash frozen in liquid nitrogen and stored at −80° C. until genomic DNA isolation.

For GUIDE-seq experiments, U2OS cells were electroporated following previously established protocols (Tsai, S. Q., et al., GUIDE-seq enables genome-wide profiling of off-target cleavage by CRISPR-Cas nucleases. Nature Biotechnology 33, 187-197 (2015); and Chen, J. S., et al., Enhanced proofreading governs CRISPR-Cas9 targeting accuracy. Nature 550, 407-410 (2017), each of which is incorporated herein by reference in its entirety). Briefly, 2×10⁵ cells per condition were transfected with 500 ng Cas9 plasmid, 250 ng sgRNA plasmid, 250 ng dRNA plasmid, and 100 pmol of an end-protected double-stranded oligonucleotide (dsODN) GUIDE-seq tag. For wells without dRNA or sgRNA, pMAX-GFP plasmid was substituted as a transfection control. 20 μl transfections were performed using a Lonza 4D nucleofector X unit and SE kit using the DN-100 program. Cells were replated in 96 well plates after transfection and harvested for genomic DNA 96 hours later.

dRNA Recutting Suppression (dReCS)

For dReCS experiments, a HEK-293T cell line with a genomically encoded BFP/GFP reporter was used (see Richardson, et al., Enhancing homology-directed genome editing by catalytically active and inactive CRISPR-Cas9 using asymmetric donor DNA. Nature Biotechnology 34, 339-344 (2016), incorporated herein by reference in its entirety). The BFP/GFP reporter HEK-293T cell line contains a BFP that is converted to GFP via HDR-mediated substitution of a single amino acid (His in BFP (CAT) to Tyr in GFP (TAT)). BFP/GFP reporter cells were plated at 3.0×10⁵ cells/well in 12-well plates. 18 hours after plating, cells were transfected with 3 μL of Turbofectin 8.0 (Origene) and 1,000 ng of total DNA. The total DNA mixture contained 272.7 ng of plasmid encoding Cas9, 54.5 ng sgRNA plasmid, 218 ng dRNA plasmid, and 454.5 ng symmetric or asymmetric single stranded donor DNA (Supplementary Data Set) (Richardson, C. D., et al., Enhancing homology-directed genome editing by catalytically active and inactive CRISPR-Cas9 using asymmetric donor DNA. Nature Biotechnology 34, 339-344 (2016), incorporated herein by reference in its entirety). For controls missing one or more of these DNA elements, the appropriate amount of DNA was replaced with a pKan-mCherry plasmid. Cells were maintained with standard passaging procedures for 4 days post-transfection until analysis by flow cytometry.

After 4 days, cells were washed with 2 mL DPBS, trypsinized with 0.5 mL 0.25% trypsin-EDTA (Life Technologies) for 2-4 minutes, and quenched with DMEM supplemented with 10% FBS. Cells were then spun down at 290×g for 4 min, aspirated, and resuspended in DPBS supplemented with 1% FBS. Cells were run through a 35 μm filter and analyzed by flow cytometry on an LSR-II flow cytometer. After gating for live cells (FSC-A vs SSC-A) and single cells (FSC-A×SSC-W), cells were analyzed for their BFP and GFP fluorescence. Gates for BFP and GFP positivity were determined by comparison to an untransfected BFP cell line. BFP+ GFP− cells were considered wildtype (WT). BFP− GFP− cells were considered to have undergone NHEJ but not HDR, as indels in this region of BFP lead to loss of fluorescence. Any cell that was GFP+ (regardless of residual BFP fluorescence) was considered to have undergone successful HDR. Percentages for each result (WT, HDR, NHEJ) were calculated as a fraction of the total cells that passed singlet gating. Percent HDR of total editing was determined as the fraction of cells with successful HDR divided by the total number of cells that underwent either HDR or NHEJ.

In Vitro Cas9 RNP Nuclease Assays

Cas9-2NLS in a pMJ915 vector (Addgene plasmid 69090) was expressed in E. coli and purified by a combination of affinity, ion exchange, and size exclusion chromatography as previously described (Anders, C. & Jinek, M. In vitro enzymology of Cas9. in Methods in Enzymology (eds. Doudna, J. A. & Sontheimer, E. J.) vol. 546 1-20 (Academic Press, 2014), incorporated herein by reference in its entirety), except the final purified protein was eluted into a buffer containing 20 mM HEPES KOH pH 7.5, 5% glycerol, 150 mM KCl, 1 mM DTT at a final concentration of 40 μM of Cas9-2NLS. FANCF sgRNA2 and FANCF dRNA1 were generated by HiScribe (NEB E2050S) T7 in vitro transcription using PCR-generated DNA as a template (Anders, C. & Jinek, M. In vitro enzymology of Cas9, in Methods in Enzymology (eds. Doudna, J. A. & Sontheimer, E. J.) vol. 546 1-20 (Academic Press, 2014), incorporated herein by reference in its entirety), (dx.doi.org/10.17504/protocols.io.dm749m). Complete sequences for all sgRNA templates can be found in Supplementary Data Set.

A 463 basepair fragment containing the on-target cut site of FANCF sgRNA2 (FANCF target site) was PCR amplified from a custom FANCF sgRNA2 target site substrate gBlock (IDT) using primers oCR1711 and oCR1712. A 329 basepair fragment containing the cut site for off-target 1 of FANCF sgRNA2 (FANCF off-target) was PCR amplified from a custom FANCF sgRNA2 off-target substrate gBlock (IDT) using oCR1713 and oCR1714 (Supplementary Data Set). Prior to nuclease experiments, sgRNA and dRNA RNP complexes were generated by incubating purified Cas9-2NLS and FANCF sgRNA2 or dRNA1 in equimolar amounts for 10 minutes. For dRNA-RNP titration experiments, 150 or 450 fmoles of FANCF-sgRNA2-RNP complex and 0, 50, 150, or 450 fmoles of dRNA-RNP Cas9-sgRNA complex were co-added to 150 fmoles of FANCF target site or FANCF off-target substrate DNA. Reaction mixtures were incubated at 37° C. for 20 minutes in 20 mM Tris, 100 mM KCl, 5 mM MgCl₂, 1 mM DTT, 0.01% Tween, 50 μg/mL Heparin. Reactions were stopped by the addition of 1:4 volume of STOP solution (8 mM Tris, 0.025% BPB, 0.025% XC, 50% Glycerol, 110 mM EDTA, 1% SDS, 3 mg/mL Proteinase K), followed by incubation at 55° C. for five minutes to liberate cut DNA fragments. Each digestion reaction was run on a 2% TAE agarose gel, post-stained with Ethidium Bromide, and resolved on a Gel-Doc (BioRad).

For pre-incubation experiments, FANCF sgRNA2 or dRNA1 RNP complexes were generated as described above. 450 fmoles of a single RNP complex was added to 150 fmoles of FANCF target site or FANCF off-target substrate DNA and incubated at 37° C. for 10 minutes. After 10 minutes, 450 fmoles of the other Cas9-RNP complex was added and allowed to incubate at 37° C. for an additional 10 minutes. Reactions were quenched, incubated, and run on a gel in an identical manner to the above experiments.

Gel densitometry analysis was performed in ImageJ. For each lane, background density was subtracted from the quantification of each band. The density of the uncut band was then divided by the total intensity of all bands in the lane to determine the uncut DNA fraction.

Genomic Editing by ciCas9

HEK-293T cells were treated according to previous methods (Rose, J. C., et al., Rapidly inducible Cas9 and DSB-ddPCR to probe editing kinetics. Nature Methods 14, 891-896 (2017), incorporated herein by reference in its entirety). Briefly, HEK-293T cells were plated in 12 well plates at 3.0×10⁵ cells/well. The day after plating, cells were transfected with 1.5 μL Turbofectin 8.0 and 500 ng of plasmid DNA. The plasmid DNA mixture contained 250 ng Cas9, 125 ng FANCF sgRNA2 sgRNA, and 125 ng dRNA. For wells without dRNA, the 125 ng of dRNA plasmid were replaced by pMAX-GFP as a transfection control.

24 hours after transfection, cells were treated with 10 μM A115 dissolved in DMSO to induce ciCas9 activity. 24 hours after treatment with A115, cells were harvested after washing with 600 μL DPBS to remove excess A115 and then resuspending cells in 600 μL ice-cold DPBS. Resuspended cells were then spun at 1,500×g for 10 min at 4° C. DPBS was aspirated and the cell pellets were stored at −80° C. until genomic DNA isolation.

Insertion and Deletion Detection by High Throughput Sequencing

Genomic DNA isolation, sequencing, and analysis were performed as previously described (Rose, J. C., et al., Rapidly inducible Cas9 and DSB-ddPCR to probe editing kinetics. Nature Methods 14, 891-896 (2017), incorporated herein by reference in its entirety). Briefly, genomic DNA was isolated using the DNEasy Blood and Tissue Kit (Qiagen) according to the manufacturer's instructions except that the proteinase K digestion was conducted for 1 hr at 56° C. 15 cycles of primary PCR to amplify the region of interest was performed using 2 μL of DNEasy eluate (˜100-300 ng template) in a 5 μL Kapa HiFi HotStart polymerase reaction (Kapa Biosystems; for primers see Supplementary Data Set). The PCR reaction was diluted with 35 μL DNAse-free water (Ambion). Illumina adapters and indexing sequences were added via 20 cycles of secondary PCR with 3 μL of diluted primary PCR product in a 10 μL Kapa Robust HotStart polymerase reaction (New England Biosciences; for primers see Supplementary Data Set). The final amplicons were run on a TBE-agarose gel (1.5%); and the product band was excised and extracted using the Freeze and Squeeze Kit according to the manufacturer's instructions (Bio-Rad). Gel-purified amplicons were quantified using Qbit dsDNA HS Assay kit (Invitrogen). Then, up to 1200 indexed amplicons were pooled, quantified by Kapa Library Quantification (Kapa Biosystems) and sequenced on a NextSeq (NextSeq 150/300 Mid V2 kit, Illumina, for primers see Supplementary Data Set).

Indels were quantified as previously described (Rose, J. C., et al., Rapidly inducible Cas9 and DSB-ddPCR to probe editing kinetics. Nature Methods 14, 891-896 (2017), incorporated herein by reference in its entirety). Briefly, after demultiplexing of reads (bcl2fastq/2.18, Illumina), indels were quantified with a custom Python script that is freely available upon request. 8-mer sequences were identified in the reference sequence located 20 bp upstream and downstream of the target sequence. Sequence distal to these 8-mers was trimmed. Reads lacking these 8-mers were discarded. For the VEGFA sgRNA3 OT2 locus, the process was the same, except 20-mer sequences located 10 bp upstream and downstream of the target sequence were used. For the VEGFA sgRNA3 OT4 locus, 8-mer sequences located 10 bp upstream and downstream of the target sequence were used. The trimmed reads were then evaluated for indels using the Python difflib package. Indels were defined as trimmed reads which differed in length from the trimmed reference and for which an insertion or deletion operation spanning or within 1 bp of the predicted Cas9 cleavage site was present. For dRNA only experiments, indels were quantified using both the sgRNA and dRNA predicted cut sites. Specificity ratios were calculated by dividing the indel percentage at the on-target locus by the indel percentage at the off-target locus for each sgRNA. For quantification of off-target editing for one of the VEGFA tru-sgRNA3 plus dRNA replicates (FIG. 2A), reads were acquired from multiple sequencing runs.

GUIDE-Seq

Calculation of indels was performed at the FANCF sgRNA2 ON and OT1 loci as described above. To determine the percentage of reads containing a dsODN tag, the same Python script as above was used and modified to count integration of the full length dsODN within 1 bp of the predicted Cas9 cleavage site. A ratio of dsODN-containing reads to indel-containing reads was calculated. To perform GUIDE-seq analysis, sample libraries were prepared as described previously (Tsai, S. Q., et al., GUIDE-seq enables genome-wide profiling of off-target cleavage by CRISPR-Cas nucleases. Nature Biotechnology 33, 187-197 (2015), incorporated herein by reference in its entirety) and sequenced on an Illumina MiSeq. Data were analyzed with the GUIDE-seq software (Tsai, S. Q., et al., Open-source guideseq software for analysis of GUIDE-seq data. Nature Biotechnology 34, 483-483 (2016), incorporated herein by reference in its entirety) allowing for up to 8 mismatches with a modification of a 35 bp window for detected off-target alignments to reference sequence. Frequency of dsODN-containing reads genome-wide were calculated per sample.

Statistical Analysis

Statistical analysis of indel frequency and specificity ratios were performed using a one-sided two sample Student's t-test.

Supplementary Data Set

TABLE 4
Primer sequences and in vitro sgRNA template sequences

| | | | SEQ |
|---|---|---|---|
| | | | ID |
| | Name | Sequence | NO: |
| 1. Site | | | |
| Specific | | | |
| Primers | Primer | Primer Sequence | |
| | BFP_ON_F | 5′-TCGTCGGCAGCGTCAGATGTGTATAAGAGACAGTGACCCTGAAGTTCATCTGC-3′ | 16 |
| | BFP_ON_R | 5′-GTCTCGTGGGCTCGGAGATGTGTATAAGAGACAGGGTCTTGTAGTTGCCGTCGT-3′ | 17 |
| | CCR5_ON_F | 5′-TCGTCGGCAGCGTCAGATGTGTATAAGAGACAGGCTCTTCAGCCTTTTGCAGT-3′ | 18 |
| | CCR5_ON_R | 5′-GTCTCGTGGGCTCGGAGATGTGTATAAGAGACAGCAGGGTGGAACAAGATGGAT-3′ | 19 |
| | CCR2_OT_F | 5′-TCGTCGGCAGCGTCAGATGTGTATAAGAGACAGAAGCACTTCAGCTTTTTGCAG-3′ | 20 |
| | CCR2_OT_R | 5′-GTCTCGTGGGCTCGGAGATGTGTATAAGAGACAGATGATTACGGTGCTCCCTGT-3′ | 21 |
| | FANCFg2_ON_F | 5′-TCGTCGGCAGCGTCAGATGTGTATAAGAGACAGCCCAGGTGCTGACGTAGGTA-3′ | 22 |
| | FANCFg2_ON_R | 5′-GTCTCGTGGGCTCGGAGATGTGTATAAGAGACAGAGCATTGCAGAGAGGCGTAT-3′ | 23 |
| | FANCFg2_OT1_F | 5′-TCGTCGGCAGCGTCAGATGTGTATAAGAGACAGCCTCCAGCCCTACTGACTGA-3′ | 24 |
| | FANCFg2_OT1_R | 5′-GTCTCGTGGGCTCGGAGATGTGTATAAGAGACAGCCCACTCTCTCCTGTTCTGG-3′ | 25 |
| | HBB_ON_F | 5′-TCGTCGGCAGCGTCAGATGTGTATAAGAGACAGGCAACCTCAAACAGACACCA-3′ | 26 |
| | HBB_ON_R | 5′-GTCTCGTGGGCTCGGAGATGTGTATAAGAGACAGGTCTCCACATGCCCAGTTTC-3′ | 27 |
| | HB B_OT 1_F | 5′-TCGTCGGCAGCGTCAGATGTGTATAAGAGACAGAGGGGAAGATCCCAGAGAAC-3′ | 28 |
| | HB B_OT 1_R | 5′-GTCTCGTGGGCTCGGAGATGTGTATAAGAGACAGTTTCCAGGCTATGCTTCCAT-3′ | 29 |
| | HBD_OT_F | 5′-TCGTCGGCAGCGTCAGATGTGTATAAGAGACAGGTTTCCATTTGCCTCCTTGA-3′ | 30 |
| | HBD_OT_R | 5′-GTCTCGTGGGCTCGGAGATGTGTATAAGAGACAGGCAACCTCAAACAGACACCA-3′ | 31 |
| | VEGFAg1_ON_F | 5′-TCGTCGGCAGCGTCAGATGTGTATAAGAGACAGGGCTCTCTGTACATGAAGCAACT-3′ | 32 |
| | VEGFAg1_ON_R | 5′-GTCTCGTGGGCTCGGAGATGTGTATAAGAGACAGCCTAGTGACTGCCGTCTGC-3′ | 33 |
| | VEGFAg1_OT1_F | 5′-TCGTCGGCAGCGTCAGATGTGTATAAGAGACAGACCTGGCCATCATCCTTCTA-3′ | 34 |
| | VEGFAg1_OT1_R | 5′-GTCTCGTGGGCTCGGAGATGTGTATAAGAGACAGCAGCAGACCCACTGAGTCAA-3′ | 35 |
| | VEGFAg1_OT4_F | 5′-TCGTCGGCAGCGTCAGATGTGTATAAGAGACAGCCTGCAGGTGTCTCCTTTTC-3′ | 36 |
| | VEGFAg1_OT4_R | 5′-GTCTCGTGGGCTCGGAGATGTGTATAAGAGACAGGCACCTGCAATGTCAGAGG-3′ | 37 |
| | VEGFAg1_OT6_F | 5′-TCGTCGGCAGCGTCAGATGTGTATAAGAGACAGCTCAGCACCTGCACTTCTTG-3′ | 38 |
| | VEGFAg1_OT6_R | 5′-GTCTCGTGGGCTCGGAGATGTGTATAAGAGACAGCAGATGTGGCCCTGAGAGAG-3′ | 39 |
| | VEGFAg1_OT11_F | 5′-TCGTCGGCAGCGTCAGATGTGTATAAGAGACAGGAGTTGTCCTGCAGCTGTACC-3′ | 40 |
| | VEGFAg1_OT11_R | 5′-GTCTCGTGGGCTCGGAGATGTGTATAAGAGACAGCAAGGCATCTCTGCCTTCAT-3′ | 41 |
| | VEGFAg2_ON_F | 5′-TCGTCGGCAGCGTCAGATGTGTATAAGAGACAGCCCCAGCTACCACCTCCT-3′ | 42 |
| | VEGFAg2_ON_R | 5′-GTCTCGTGGGCTCGGAGATGTGTATAAGAGACAGGAACAGCCCAGAAGTTGGAC-3′ | 43 |
| | VEGFAg2_OT1_F | 5′-TCGTCGGCAGCGTCAGATGTGTATAAGAGACAGGCAGTACTCCCTGCTGTCCT-3′ | 44 |
| | VEGFAg2_OT1_R | 5′-GTCTCGTGGGCTCGGAGATGTGTATAAGAGACAGGGTTTCCCAATTTCATCTTCA-3′ | 45 |
| | VEGFAg2_OT2_F | 5′-TCGTCGGCAGCGTCAGATGTGTATAAGAGACAGGCAGCCTATTGTCTCCTGGT-3′ | 46 |
| | VEGFAg2_OT2_R | 5′-GTCTCGTGGGCTCGGAGATGTGTATAAGAGACAGCTTGCCTGTAAGGCCACAGT-3′ | 47 |
| | VEGFAg2_OT17_F | 5′-TCGTCGGCAGCGTCAGATGTGTATAAGAGACAGCTCCCATGAGGGGTTTGAGT-3′ | 48 |
| | VEGFAg2_OT17_R | 5′-GTCTCGTGGGCTCGGAGATGTGTATAAGAGACAGTTGCACAAGAACCTGCTGTC-3′ | 49 |
| | VEGFAg2_OT19_F | 5′-TCGTCGGCAGCGTCAGATGTGTATAAGAGACAGCATTTGTCCAGGAACCCCTA-3′ | 50 |
| | VEGFAg2_OT19_R | 5′-GTCTCGTGGGCTCGGAGATGTGTATAAGAGACAGGCTTTGGGCTTTTAGCCTCT-3′ | 51 |
| | VEGFAg3_ON_F | 5′-TCGTCGGCAGCGTCAGATGTGTATAAGAGACAGGCAGCGTCTTCGAGAGTGA-3′ | 52 |
| | VEGFAg3_ON_R | 5′-GTCTCGTGGGCTCGGAGATGTGTATAAGAGACAGGAACAGCCCAGAAGTTGGAC-3′ | 53 |
| | VEGFAg3_OT1_F | 5′-TCGTCGGCAGCGTCAGATGTGTATAAGAGACAGAGGGACCCCTCTGACAGACT-3′ | 54 |
| | VEGFAg3_OT1_R | 5′-GTCTCGTGGGCTCGGAGATGTGTATAAGAGACAGCAGCCCTCAGACTTCACATT-3′ | 55 |
| | VEGFAg3_OT2_F | 5′-TCGTCGGCAGCGTCAGATGTGTATAAGAGACAGGAGGGGAAGGGGTGAAGG-3′ | 56 |
| | VEGFAg3_OT2_R | 5′-GTCTCGTGGGCTCGGAGATGTGTATAAGAGACAGGCAGTGAGGAGGTGGTTCTT-3′ | 57 |
| | VEGFAg3_OT4_F | 5′-TCGTCGGCAGCGTCAGATGTGTATAAGAGACAGGGCCCATTTCTCCTTTGA-3′ | 58 |
| | VEGFAg3_OT4_R | 5′-GTCTCGTGGGCTCGGAGATGTGTATAAGAGACAGTTAGGAGAGCTGGCTTGGAA-3′ | 59 |
| | VEGFAg3_OT18_F | 5′-TCGTCGGCAGCGTCAGATGTGTATAAGAGACAGGGGAATCTAATGTATGGCATGG-3′ | 60 |
| | VEGFAg3_OT18_R | 5′-GTCTCGTGGGCTCGGAGATGTGTATAAGAGACAGCGTATTCAGGGTGTGCAATG-3′ | 61 |
| | ZSCAN2g1_ON_F | 5′-TCGTCGGCAGCGTCAGATGTGTATAAGAGACAGGCTCCCAGCTCGTAGTGC-3′ | 62 |
| | ZSCAN2g1_ON_R | 5′-GTCTCGTGGGCTCGGAGATGTGTATAAGAGACAGGTTCCAGCTAAAGCCTTTCC-3′ | 63 |
| | ZSCAN2g1_OT1_F | 5′-TCGTCGGCAGCGTCAGATGTGTATAAGAGACAGCCCACATGTACCACATTTGT-3′ | 64 |
| | ZSCAN2g1_OT1_R | 5′-GTCTCGTGGGCTCGGAGATGTGTATAAGAGACAGCCGTATCAGTGTGATGCATGT-3′ | 65 |
| | ZSCAN2g1_OT2_F | 5′-TCGTCGGCAGCGTCAGATGTGTATAAGAGACAGTGTGGCACAAAGTGGAAGAG-3′ | 66 |
| | ZSCAN2g1_OT2_R | 5′-GTCTCGTGGGCTCGGAGATGTGTATAAGAGACAGCAGAGCCTTTATTGCCCATC-3′ | 67 |
| 2. Indexing | | | |
| PCR | | | |
| primers | Primer | Primer Sequence | |
| | Index_F1 | 5′-AATGATACGGCGACCACCGAGATCTACACGCTAACTATCGTCGGCAGCGTC-3′ | 68 |
| | Index_F2 | 5′-AATGATACGGCGACCACCGAGATCTACACCACGGGGCTCGTCGGCAGCGTC-3′ | 69 |
| | Index_F3 | 5′-AATGATACGGCGACCACCGAGATCTACACAATACCAGTCGTCGGCAGCGTC-3′ | 70 |
| | Index_F4 | 5′-AATGATACGGCGACCACCGAGATCTACACAGTCTCGCTCGTCGGCAGCGTC-3′ | 71 |
| | Index_F5 | 5′-AATGATACGGCGACCACCGAGATCTACACTCGTCAGCTCGTCGGCAGCGTC-3′ | 72 |
| | Index_F6 | 5′-AATGATACGGCGACCACCGAGATCTACACGAGCTTAATCGTCGGCAGCGTC-3′ | 73 |
| | Index_F7 | 5′-AATGATACGGCGACCACCGAGATCTACACGCCATTGTTCGTCGGCAGCGTC-3′ | 74 |
| | Index_F8 | 5′-AATGATACGGCGACCACCGAGATCTACACATGCCAGATCGTCGGCAGCGTC-3′ | 75 |
| | Index_F9 | 5′-AATGATACGGCGACCACCGAGATCTACACCTGTGCTTTCGTCGGCAGCGTC-3′ | 76 |
| | Index_F10 | 5′-AATGATACGGCGACCACCGAGATCTACACGGAGTCAATCGTCGGCAGCGTC-3′ | 77 |
| | Index_F11 | 5′-AATGATACGGCGACCACCGAGATCTACACAGAACAACTCGTCGGCAGCGTC-3′ | 78 |
| | Index_F12 | 5′-AATGATACGGCGACCACCGAGATCTACACCACACCATTCGTCGGCAGCGTC-3′ | 79 |
| | Index_F13 | 5′-AATGATACGGCGACCACCGAGATCTACACAGCGATTTTCGTCGGCAGCGTC-3′ | 80 |
| | Index_F14 | 5′-AATGATACGGCGACCACCGAGATCTACACGACGCGGCTCGTCGGCAGCGTC-3′ | 81 |
| | Index_F15 | 5′-AATGATACGGCGACCACCGAGATCTACACTGGCCTGTTCGTCGGCAGCGTC-3′ | 82 |
| | Index_F16 | 5′-AATGATACGGCGACCACCGAGATCTACACGAGCGACATCGTCGGCAGCGTC-3′ | 83 |
| | Index_F17 | 5′-AATGATACGGCGACCACCGAGATCTACACAAAGCTTTTCGTCGGCAGCGTC-3′ | 84 |
| | Index_F18 | 5′-AATGATACGGCGACCACCGAGATCTACACAATGGGAATCGTCGGCAGCGTC-3′ | 85 |
| | Index_F19 | 5′-AATGATACGGCGACCACCGAGATCTACACTCCCGTAATCGTCGGCAGCGTC-3′ | 86 |
| | Index_F20 | 5′-AATGATACGGCGACCACCGAGATCTACACTCTTCAAATCGTCGGCAGCGTC-3′ | 87 |
| | Index_F21 | 5′-AATGATACGGCGACCACCGAGATCTACACATTCTCAATCGTCGGCAGCGTC-3′ | 88 |
| | Index_F22 | 5′-AATGATACGGCGACCACCGAGATCTACACCCTGCTTTTCGTCGGCAGCGTC-3′ | 89 |
| | Index_F23 | 5′-AATGATACGGCGACCACCGAGATCTACACACTAAGCGTCGTCGGCAGCGTC-3′ | 90 |
| | Index_F24 | 5′-AATGATACGGCGACCACCGAGATCTACACCTGGGTCCTCGTCGGCAGCGTC-3′ | 91 |
| | Index_F25 | 5′-AATGATACGGCGACCACCGAGATCTACACTACTCCAGTCGTCGGCAGCGTC-3′ | 92 |
| | Index_R1 | 5′-CAAGCAGAAGACGGCATACGAGATTACGAAGTCGTCTCGTGGGCTCGG-3′ | 93 |
| | Index_R2 | 5′-CAAGCAGAAGACGGCATACGAGATGACGAGATTGTCTCGTGGGCTCGG-3′ | 94 |
| | Index_R3 | 5′-CAAGCAGAAGACGGCATACGAGATACCGTAAGAGTCTCGTGGGCTCGG-3′ | 95 |
| | Index_R4 | 5′-CAAGCAGAAGACGGCATACGAGATTAGTGGCAAGTCTCGTGGGCTCGG-3′ | 96 |
| | Index_R5 | 5′-CAAGCAGAAGACGGCATACGAGATCATTAACGCGTCTCGTGGGCTCGG-3′ | 97 |
| | Index_R6 | 5′-CAAGCAGAAGACGGCATACGAGATTCGTTGAAGGTCTCGTGGGCTCGG-3′ | 98 |
| | Index_R7 | 5′-CAAGCAGAAGACGGCATACGAGATTAGTACGCTGTCTCGTGGGCTCGG-3′ | 99 |
| | Index_R8 | 5′-CAAGCAGAAGACGGCATACGAGATCTCAGATCAGTCTCGTGGGCTCGG-3′ | 100 |
| | Index_R9 | 5′-CAAGCAGAAGACGGCATACGAGATTTCACCGTAGTCTCGTGGGCTCGG-3′ | 101 |
| | Index_R10 | 5′-CAAGCAGAAGACGGCATACGAGATGTCATGCATGTCTCGTGGGCTCGG-3′ | 102 |
| | Index_R11 | 5′-CAAGCAGAAGACGGCATACGAGATAGGACAGTTGTCTCGTGGGCTCGG-3′ | 103 |
| | Index_R12 | 5′-CAAGCAGAAGACGGCATACGAGATATGGTGTCTGTCTCGTGGGCTCGG-3′ | 104 |
| | Index_R13 | 5′-CAAGCAGAAGACGGCATACGAGATGGATGTTCTGTCTCGTGGGCTCGG-3′ | 105 |
| | Index_R14 | 5′-CAAGCAGAAGACGGCATACGAGATCTTATCCAGGTCTCGTGGGCTCGG-3′ | 106 |
| | Index_R15 | 5′-CAAGCAGAAGACGGCATACGAGATGTAAGTCACGTCTCGTGGGCTCGG-3′ | 107 |
| | Index_R16 | 5′-CAAGCAGAAGACGGCATACGAGATTTCAGTGAGGTCTCGTGGGCTCGG-3′ | 108 |
| | Index_R17 | 5′-CAAGCAGAAGACGGCATACGAGATCTCGTAATGGTCTCGTGGGCTCGG-3′ | 109 |
| | Index_R18 | 5′-CAAGCAGAAGACGGCATACGAGATCATGTCTCAGTCTCGTGGGCTCGG-3′ | 110 |
| | Index_R19 | 5′-CAAGCAGAAGACGGCATACGAGATAATCGTGGAGTCTCGTGGGCTCGG-3′ | 111 |
| | Index_R20 | 5′-CAAGCAGAAGACGGCATACGAGATGTATCAGTCGTCTCGTGGGCTCGG-3′ | 112 |
| | Index_R21 | 5′-CAAGCAGAAGACGGCATACGAGATAGCAGATGTGTCTCGTGGGCTCGG-3′ | 113 |
| | Index_R22 | 5′-CAAGCAGAAGACGGCATACGAGATTCCTAACGTGTCTCGTGGGCTCGG-3′ | 114 |
| | Index_R23 | 5′-CAAGCAGAAGACGGCATACGAGATAACAGTCCAGTCTCGTGGGCTCGG-3′ | 115 |
| | Index_R24 | 5′-CAAGCAGAAGACGGCATACGAGATCCTTGAGAAGTCTCGTGGGCTCGG-3′ | 116 |
| | Index_R25 | 5′-CAAGCAGAAGACGGCATACGAGATTTAAGCCTGGTCTCGTGGGCTCGG-3′ | 117 |
| | Index_R26 | 5′-CAAGCAGAAGACGGCATACGAGATTTAGACCACGTCTCGTGGGCTCGG-3′ | 118 |
| | Index_R27 | 5′-CAAGCAGAAGACGGCATACGAGATTGTCTAGTGGTCTCGTGGGCTCGG-3′ | 119 |
| | Index_R28 | 5′-CAAGCAGAAGACGGCATACGAGATTAGATCGAGGTCTCGTGGGCTCGG-3′ | 120 |
| | Index_R29 | 5′-CAAGCAGAAGACGGCATACGAGATTGAATGCCAGTCTCGTGGGCTCGG-3′ | 121 |
| | Index_R30 | 5′-CAAGCAGAAGACGGCATACGAGATGTGCAATGTGTCTCGTGGGCTCGG-3′ | 122 |
| | Index_R31 | 5′-CAAGCAGAAGACGGCATACGAGATAGTGGCATAGTCTCGTGGGCTCGG-3′ | 123 |
| | Index_R32 | 5′-CAAGCAGAAGACGGCATACGAGATATGATCGGTGTCTCGTGGGCTCGG-3′ | 124 |
| | Index_R33 | 5′-CAAGCAGAAGACGGCATACGAGATAGTCTACCTGTCTCGTGGGCTCGG-3′ | 125 |
| | Index_R34 | 5′-CAAGCAGAAGACGGCATACGAGATGATCAACTGGTCTCGTGGGCTCGG-3′ | 126 |
| | Index_R35 | 5′-CAAGCAGAAGACGGCATACGAGATATCGGTAGTGTCTCGTGGGCTCGG-3′ | 127 |
| | Index_R36 | 5′-CAAGCAGAAGACGGCATACGAGATCGTATGATGGTCTCGTGGGCTCGG-3′ | 128 |
| | Index_R37 | 5′-CAAGCAGAAGACGGCATACGAGATTTACTGACGGTCTCGTGGGCTCGG-3′ | 129 |
| | Index_R38 | 5′-CAAGCAGAAGACGGCATACGAGATCTGTCGTAAGTCTCGTGGGCTCGG-3′ | 130 |
| | Index_R39 | 5′-CAAGCAGAAGACGGCATACGAGATTCAACTGGTGTCTCGTGGGCTCGG-3′ | 131 |
| | Index_R40 | 5′-CAAGCAGAAGACGGCATACGAGATATCGATCTCGTCTCGTGGGCTCGG-3′ | 132 |
| | Index_R41 | 5′-CAAGCAGAAGACGGCATACGAGATGCAACTATGGTCTCGTGGGCTCGG-3′ | 133 |
| | Index_R42 | 5′-CAAGCAGAAGACGGCATACGAGATGATGACTTCGTCTCGTGGGCTCGG-3′ | 134 |
| | Index_R43 | 5′-CAAGCAGAAGACGGCATACGAGATGACGTTACAGTCTCGTGGGCTCGG-3′ | 135 |
| | Index_R44 | 5′-CAAGCAGAAGACGGCATACGAGATCATCTGCTAGTCTCGTGGGCTCGG-3′ | 136 |
| | Index_R45 | 5′-CAAGCAGAAGACGGCATACGAGATATTAGTCGGGTCTCGTGGGCTCGG-3′ | 137 |
| | Index_R46 | 5′-CAAGCAGAAGACGGCATACGAGATTAGCGTACTGTCTCGTGGGCTCGG-3′ | 138 |
| | Index_R47 | 5′-CAAGCAGAAGACGGCATACGAGATCCAAGCAATGTCTCGTGGGCTCGG-3′ | 139 |
| | Index_R48 | 5′-CAAGCAGAAGACGGCATACGAGATCCGTAATTGGTCTCGTGGGCTCGG-3′ | 140 |
| 3. in vitro | | | |
| sgRNA | | | |
| templates | Template | Sequence (cut site underlined) | |
| | FANCF sgRNA2 | GGTTCTCCAGCAGGCGCAGAGAGAGCAGGACGTCACAGTGACCGAGGGCCTGGAAGTT | 141 |
| | target site | CGCTAATCCCGGAACTGGACCCCGCCCAAAGCCGCCCTCTTGCCTCCACTGGTTGTGCAG | |
| | substrate | CCGCCGCTCCAGAGCCGTGCGAATGGGGCCATGCCGACCAAAGCGCCGATGGATGTGGC | |
| | | GCAGGTAGCGCGCCCACTGCAAGGCCCGGCGCACGGTGGCGGGGTCCCAGGTGCTGAC | |
| | | GTAGGTAGTGCTTGAGACCGCCAGAAGCTCGGAAAAGCGATCCAGGTGCTGCAGAAGG | |
| | | GATTCCATGAGGTGCGCGAAGGCCCTACTTCCGCTTTCACCTTGGAGACGGCGACTCTCT | |
| | | GCGTACTGATTGGAACATCCGCGAAATGATACGCCTCTCTGCAATGCTATTGGTCGAAA | |
| | | TGCATGTCAATCTCCCAGCGTCTTTATCCGTGTTCCTTGACTCTGGGCAACCCTG | |
| | | TCTCCCACTCTCTCCTGTTCTGGCTCCCTTGTTTTTTCTCCCTCCTCTCTCTTCCACC | |
| | | GAGTTACCAGCCTCTGTCTCACCTCATCCACTATGCTGCAGAAGGGATTCCAAGGGGAA | |
| | FANCF sgRNA2 | TACGAAGTCAGTCATATGAAACCCAGGCACCTCTGTCAGTCAGTAGGGCTGGAGGTGGA | 142 |
| | off-target | GACAGAAATGGGGCCCCAGATGGGATCTCTGAGGCAGCCCTTTGAGATGAGTCCCACAA | |
| | substrate | GATCAAGAACATCCCTCCCACCCCATTCATTCCAGGCCCGGGATGAACTATCACGATCCT | |
| | | GAAACAGTTCAAATCTCAGCACCTCACGGG | |
| | FANCF sgRNA2 | ggatcctaatacgactcactataGCTGCAGAAGGGATTCCATGgttttagagctag | 143 |
| | template* | aaatagcaagttaaaataaggctagtccgttatcaacttgaaaaagtggcaccgagt | |
| | | cggtgctttttt | |
| | FANCF dRNA1 | ggatcctaatacgactcactataGAAGGGATTCCAAGgttttagagctagaaatagcaa | 144 |
| | template* | gttaaaataaggctagtccgttatcaacttgaaaaagtggcaccgagtcggtgctttttt | |
| | oCR1711 | GGTTCTCCAGCAGGCGCAG | 145 |
| | oCR1712 | GTTGCCCAGAGTCAAGGAACACG | 146 |
| | oCR1713 | CCTGTCTCCCACTCTCTCCTG | 147 |
| | oCR1714 | CCCGTGAGGTGCTGAGATTTG | 148 |

*lower case: sequence for the tracrRNA/sgRNA backbone; upper case: sequence targeting sequence of the guide RNA or dead-RNA

TABLE 5
dRNA sequences

| | | | | Targeting | | | |
|---|---|---|---|---|---|---|---|
| | | | | Sequence | | | |
| | | | | (lower | | | |
| | | | | case g | | | Targeting |
| | | | | indicates | | | sequence |
| | | | | 5′ | | | length |
| | | | | mismatch | SEQ | Targeting | (minus |
| | | | | to OT | ID | sequence | mismatch 5′ |
| Gene | sgRNA | OT | dRNA | site) | NO: | length | nucleotide) |
| CCR5 | R-30 | CCR2 | dRNA1 | GCTGGTGTTCATCTT | 149 | 15 | 15 |
| CCR5 | R-30 | CCR2 | dRNA2 | gCACCAGCGAGTAGAG | 150 | 16 | 15 |
| CCR5 | R-30 | CCR2 | dRNA3 | gCAGCGAGTAGAGCGG | 151 | 16 | 15 |
| CCR5 | R-30 | CCR2 | dRNA4 | GCGGAGGCAGGAGTT | 152 | 15 | 15 |
| CCR5 | R-30 | CCR2 | dRNA5 | GACGTGAAGCAAATTG | 153 | 16 | 16 |
| CCR5 | R-30 | CCR2 | dRNA5s | gCGTGAAGCAAATTG | 154 | 15 | 14 |
| CCR5 | R-30 | CCR2 | dRNA6 | gCTCCGCTCTACTCGC | 155 | 16 | 15 |
| HBB | G10 | OT1 | dRNA4 | gCCTTACTGCCCTGTG | 156 | 16 | 15 |
| HBB | G10 | OT1 | dRNA5 | GCCCTTACTGCCCTGT | 157 | 16 | 16 |
| HBB | G10 | OT1 | dRNA6 | GCCCTTACTGCCCTG | 158 | 15 | 15 |
| HBB | G10 | OT1 | dRNA7 | GCCCCACAGGGCAGTA | 159 | 16 | 16 |
| HBB | R-01/R-03/R-04 | HBD | dRNA1 | GAACGTGGATGCAGT | 160 | 15 | 15 |
| HBB | R-01/R-03/R-04 | HBD | dRNA2 | GTGGATGCAGTTGG | 161 | 14 | 14 |
| HBB | R-01/R-03/R-04 | HBD | dRNA4 | GCTGTCAATGCCCTG | 162 | 15 | 15 |
| HBB | R-01/R-03/R-04 | HBD | dRNA5 | gCCCACAGGGCAGTAA | 163 | 16 | 15 |
| HBB | R-01/R-03/R-04 | HBD | dRNA6 | GCCGTTACTGCCCTG | 164 | 15 | 15 |
| HBB | R-01/R-03/R-04 | HBD | dRNA7 | gTCACTTTGCCCCACA | 165 | 16 | 15 |
| FANCF | sgRNA2 | OT1 | dRNA1 | GAAGGGATTCCAAG | 166 | 14 | 14 |
| FANCF | sgRNA2 | OT1 | dRNA2 | GACTTCGTATTCCCCT | 167 | 16 | 16 |
| FANCF | sgRNA2 | OT1 | dRNA3 | GCAGCATAGTGGATG | 168 | 15 | 15 |
| FANCF | sgRNA2 | OT1 | dRNA5 | GCAGAAGGGATTCCA | 169 | 15 | 15 |
| FANCF | sgRNA2 | OT1 | dRNA4 | GCAGAAGGGATTCCAA | 170 | 16 | 16 |
| VEGFA | sgRNA1 | OT1 | dRNA2 | GGATTTGTGGGATGGA | 171 | 16 | 16 |
| VEGFA | sgRNA1 | OT1 | dRNA7 | GGAGGGAGTTTGCTCC | 172 | 16 | 16 |
| VEGFA | sgRNA1 | OT1 | dRNA7s | GAGGGAGTTTGCTCC | 173 | 15 | 15 |
| VEGFA | sgRNA1 | OT1 | dRNA9 | GGGAGTTTGCTCCTG | 174 | 15 | 15 |
| VEGFA | sgRNA1 | OT4 | dRNA1 | GGGTGGAGTTTGCTCC | 175 | 16 | 16 |
| VEGFA | sgRNA1 | OT4 | dRNAls | GGTGGAGTTTGCTCC | 176 | 15 | 15 |
| VEGFA | sgRNA1 | OT4 | dRNA2 | gTTATGATAGGGAGGG | 177 | 16 | 15 |
| VEGFA | sgRNA1 | OT4 | dRNA3 | GCCCATTATGATAGGG | 178 | 16 | 16 |
| VEGFA | sgRNA1 | OT4 | dRNA4 | GTGGAGTTTGCTCCT | 179 | 15 | 15 |
| VEGFA | sgRNA1 | OT4 | dRNA5 | GGAGTTTGCTCCTG | 180 | 14 | 14 |
| VEGFA | sgRNA1 | OT4 | dRNA6 | GTTTGCTCCTGGGGA | 181 | 15 | 15 |
| VEGFA | sgRNA1 | OT4 | dRNA8 | GGCCCTTCCATCCCC | 182 | 15 | 15 |
| VEGFA | sgRNA1 | OT6 | dRNA1 | GAGGGAGTTTGCTCC | 183 | 15 | 15 |
| VEGFA | sgRNA1 | OT6 | dRNA2 | gTCCCATCACGGGGGA | 184 | 16 | 15 |
| VEGFA | sgRNA1 | OT6 | dRNA4 | GAGGCTCCCATCACGG | 185 | 16 | 16 |
| VEGFA | sgRNA1 | OT6 | dRNA4s | GGCTCCCATCACGG | 186 | 14 | 14 |
| VEGFA | sgRNA1 | OT6 | dRNA5 | GGGAGTTTGCTCCT | 187 | 14 | 14 |
| VEGFA | sgRNA1 | OT6 | dRNA6 | GGGAGTTTGCTCCTG | 188 | 15 | 15 |
| VEGFA | sgRNA1 | OT6 | dRNA8 | GATCACAGGTTCCCC | 189 | 15 | 15 |
| VEGFA | sgRNA1 | OT11 | dRNA1 | GGGGAAGTTTGCTCC | 190 | 15 | 15 |
| VEGFA | sgRNA1 | OT11 | dRNA2 | GCTCCTGGCATTCAGT | 191 | 16 | 16 |
| VEGFA | sgRNA1 | OT11 | dRNA3 | GCTCCTGGCATTCAG | 192 | 15 | 15 |
| VEGFA | sgRNA1 | OT11 | dRNA4 | GTCACAACTCGGGGAG | 193 | 16 | 16 |
| VEGFA | sgRNA1 | OT11 | dRNA5 | GTCACAACTCGGGGA | 194 | 15 | 15 |
| VEGFA | sgRNA1 | OT11 | dRNA6 | GTCACAACTCGGGG | 195 | 14 | 14 |
| VEGFA | sgRNA1 | OT11 | dRNA7 | GCTGTCACAACTCG | 196 | 14 | 14 |
| VEGFA | sgRNA1 | OT11 | dRNA8 | gTACCCACTGAATGCC | 197 | 16 | 15 |
| VEGFA | sgRNA2 | OT1 | dRNA1 | gCCCCCACCCCGCCCC | 198 | 16 | 15 |
| VEGFA | sgRNA2 | OT1 | dRNA2 | GGTGGGGGGGGTCTTT | 199 | 16 | 16 |
| VEGFA | sgRNA2 | OT1 | dRNA2s | GTGGGGGGGGTCTTT | 200 | 15 | 15 |
| VEGFA | sgRNA2 | OT1 | dRNA3 | GGCTGCTGTTGCAG | 201 | 14 | 14 |
| VEGFA | sgRNA2 | OT1 | dRNA4 | GGGGGCGGGGTGGGGG | 202 | 16 | 16 |
| VEGFA | sgRNA2 | OT2 | dRNA2 | gCCAAGGCGCTCCTAG | 203 | 16 | 15 |
| VEGFA | sgRNA2 | OT2 | dRNA3 | gTCTGGCCAAGTTTTG | 204 | 16 | 15 |
| VEGFA | sgRNA2 | OT2 | dRNA4 | GGTGGAGGGGCCCCT | 205 | 15 | 15 |
| VEGFA | sgRNA2 | OT2 | dRNA6 | GCCAGAGGCGGGGTGG | 206 | 16 | 16 |
| VEGFA | sgRNA2 | OT2 | dRNA7 | GGCCAGAGGCGGGG | 207 | 14 | 14 |
| VEGFA | sgRNA2 | OT2 | dRNA8 | gACTTGGCCAGAGGCG | 208 | 16 | 15 |
| VEGFA | sgRNA2 | OT17 | dRNA1 | gCCCCCACCCCGCCTC | 209 | 16 | 15 |
| VEGFA | sgRNA2 | OT17 | dRNA2 | GTTGGACGTCCTGAGG | 210 | 16 | 16 |
| VEGFA | sgRNA2 | OT17 | dRNA8 | GTCCAACAGGGTTG | 211 | 14 | 14 |
| VEGFA | sgRNA2 | OT19 | dRNA1 | gCCCCCACCCCGCCTC | 212 | 16 | 15 |
| VEGFA | sgRNA2 | OT19 | dRNA2 | GGTTTATTCTTTCCTG | 213 | 16 | 16 |
| VEGFA | sgRNA2 | OT19 | dRNA3 | gTGAGGCGGGGTGGGG | 214 | 16 | 15 |
| VEGFA | sgRNA2 | OT19 | dRNA3s | GAGGCGGGGTGGGG | 215 | 14 | 14 |
| VEGFA | sgRNA2 | OT19 | dRNA4 | gCTGAGGCGGGGTGGG | 216 | 16 | 15 |
| VEGFA | sgRNA2 | OT19 | dRNA5 | gTTCCTGAGGCGGGGT | 217 | 16 | 15 |
| VEGFA | sgRNA2 | OT19 | dRNA6 | GAATAAACCTCATACC | 218 | 16 | 16 |
| VEGFA | sgRNA3 | OT2 | dRNA1 | GTGAGTGTGTGTGTG | 219 | 15 | 15 |
| VEGFA | sgRNA3 | OT2 | dRNA2 | GAGTGAGTGTGTGTGT | 220 | 16 | 16 |
| VEGFA | sgRNA3 | OT2 | dRNA5 | GTGTGTGGGGGGGACT | 221 | 16 | 16 |
| VEGFA | sgRNA3 | OT4 | dRNA1 | GTGAGTGTATGCGTG | 222 | 15 | 15 |
| VEGFA | sgRNA3 | OT4 | dRNA2 | GCGTGTGGCTTTAGC | 223 | 15 | 15 |
| VEGFA | sgRNA3 | OT4 | dRNA3 | GCGTGTGGCTTTAG | 224 | 14 | 14 |
| VEGFA | sgRNA3 | OT4 | dRNA4 | GGCTTTAGCGGGAAGC | 225 | 16 | 16 |
| VEGFA | sgRNA3 | OT4 | dRNA4s | GCTTTAGCGGGAAGC | 226 | 15 | 15 |
| VEGFA | sgRNA3 | OT18 | dRNA2 | gCCACCTTTTATGTGT | 227 | 16 | 15 |
| VEGFA | sgRNA3 | OT18 | dRNA3 | gACCACCTTTTATGTG | 228 | 16 | 15 |
| VEGFA | sgRNA3 | OT18 | dRNA4 | gTCACCCACACATAAA | 229 | 16 | 15 |
| VEGFA | sgRNA3 | OT18 | dRNA5 | gCCCACACATAAAAGG | 230 | 16 | 15 |
| ZSCAN2 | sgRNA1 | OT1 | dRNA1 | GGCAAGGGCTTCAGCC | 231 | 16 | 16 |
| ZSCAN2 | sgRNA1 | OT1 | dRNA1s | GCAAGGGCTTCAGCC | 232 | 15 | 15 |
| ZSCAN2 | sgRNA1 | OT1 | dRNA2 | GGCCTCAAATCTTC | 233 | 14 | 14 |
| ZSCAN2 | sgRNA1 | OT1 | dRNA3 | GTGAGGAGTGTGGCAA | 234 | 16 | 16 |
| ZSCAN2 | sgRNA1 | OT1 | dRNA4 | GAAGATTTGAGGCC | 235 | 14 | 14 |
| ZSCAN2 | sgRNA1 | OT2 | dRNA1 | GGGAAGAGCTTCAGCA | 236 | 16 | 16 |
| ZSCAN2 | sgRNA1 | OT2 | dRNA1s | GGAAGAGCTTCAGCA | 237 | 15 | 15 |
| ZSCAN2 | sgRNA1 | OT2 | dRNA2 | GAAGCTCTTCCCTCAC | 238 | 16 | 16 |
| ZSCAN2 | sgRNA1 | OT2 | dRNA3 | GCTCTTCCCTCACA | 239 | 14 | 14 |
| ZSCAN2 | sgRNA1 | OT2 | dRNA4 | GAGCTGTTCCCTGTGA | 240 | 16 | 16 |
| dReCS | | | | | | | |
| dRNAs | | | | | | | |

| | | | | | | |
|---|---|---|---|---|---|---|
| BFP | sgRNA1/sgRNA2 | dRNA1 | GCACGCCATAGGTCA | 241 | 15 | 15 |
| BFP | sgRNA1/sgRNA2 | dRNA2 | GCACGCCATAGGTC | 242 | 14 | 14 |
| BFP | sgRNA1/sgRNA2 | dRNA3 | GCCATAGGTCAGGG | 243 | 14 | 14 |

TABLE 6
sgRNA sequences

| | | | SEQ | | |
|---|---|---|---|---|---|
| | | Target | ID | | |
| GENE | sgRNA | sequence | NO: | PAM | Reference(s) |
| CCR5 | R30 | GTAGAGCGGAG | 244 | GGG | Cradick, T. J., et al., |
| | | GCAGGAGGC | | | Nucleic Acids Res. |
| | | | | | 41.9584-9592(2013). |
| FANCF | sgRNA2 | GCTGCAGAAGG | 245 | AGG | Kleinstiver, B. P., et al., |
| | | GATTCCATG | | | Nature, 529(7587), |
| | | | | | 490-495 (2016); |
| | | | | | Chen, J. S., |
| | | | | | et al., |
| | | | | | Nature 550, 407-410 (2017). |
| HBB | GIO | gCTTGCCCCAC | 246 | CGG | DeWitt, M. A., et al., |
| | | AGGGCAGTAA | | | Sci. Transl. Med. 8, |
| | | | | | 360ra134-360ra 134 (2016). |
| HBB | ROl | GTGAACGTGGA | 247 | tGG | Cradick, T. J., et al., |
| | | TGAAGTTGG | | | Nucleic Acids Res. |
| | | | | | 41.9584-9592(2013). |
| HBB | R03 | gACGTTCACCT | 248 | gGG | Cradick, T. J., et al., |
| | | TGCCCCACA | | | Nucleic Acids Res. |
| | | | | | 41,9584-9592(2013). |
| HBB | R04 | gCACGTTCACC | 249 | aGG | Cradick, T. J., et al., |
| | | TTGCCCCAC | | | Nucleic Acids Res. |
| | | | | | 41,9584-9592(2013). |
| VEGFA | sgRNA 1 | GGGTGGGGGGA | 250 | TGG | Fu. Y., et al., |
| | | GTTTGCTCC | | | Nat. Biotechnol. 31, 822- |
| | | | | | 826(2013). |
| VEGFA | sgRNA2 | GACCCCCTCCA | 251 | CGG | Fu. Y., et al., |
| | | CCCCGCCTC | | | Nat. Biotechnol. 31. |
| | | | | | 822-826 (2013); |
| | | | | | Kleinstiver, B. P., et al., |
| | | | | | Nature. 529(7587), |
| | | | | | 490-495 (2016); |
| | | | | | Chen, J. S., et al., |
| | | | | | Nature 550, 407-10 |
| | | | | | (2017). |
| VEGFA | sgRNA3 | GGTGAGTGAG | 252 | TGG | Fu, Y., et al., |
| | | TGTGTGCGTG | | | Nat. Biotechnol. 31, |
| | | | | | 822-826 (2013); |
| | | | | | Kleinstiver, B. P., et al., |
| | | | | | Nature. 529(7587), |
| | | | | | 490-495 (2016); |
| | | | | | Slaymaker, I. M., et al., |
| | | | | | Science 351, |
| | | | | | 84-88 (2016); |
| | | | | | Chen. J. S., et al., |
| | | | | | Nature 550, |
| | | | | | 407-410(2017). |
| VEGFA | tru- | GAGTGAGTGT | 253 | TGG | Fu, Y., et al., |
| | sgRNA3 | GTGCGTG | | | Nat. Biotechnol. 32(3), |
| | | | | | 279-284 (2014). |
| ZSCAN2 | sgRNA 1 | GTGCGGCAAGA | 254 | GGG | Kleinstiver, B. P., et al., |
| | | GCTTCAGCC | | | Nature. |
| | | | | | 529(7587), 490-495 (2016). |
| Non- | | | | | |
| genomic | | Target | | | |
| GENE | sgRNA | sequence | | PAM | Reference(s) |
| BFP | sgRNA 1 | GCTGAAGCAC | 255 | GGG | Richardson, C. D.. et al., |
| | | TGCACGCCAT | | | Nat. Biotechnol. 34, |
| | | | | | 339-344 (2016). |

TABLE 7
On Target Loci. “*” indicates
hg38 coordinates

| | | | SEQ | | | | |
|---|---|---|---|---|---|---|---|
| | | Target | ID | | | | |
| Gene | sgRNA | Sequence | NO: | PAM | Chr | Start* | End* |
| CCR5 | R30 | GTAGAGC | 256 | GGG | chr | 46372991 | 46373013 |
| | | GGAGGCA | | | 3 | | |
| | | GGAGGC | | | | | |
| FANCF | sgRNA2 | GCTGCAG | 257 | AGG | chr | 22625792 | 22625814 |
| | | AAGGGAT | | | 11 | | |
| | | TCCATG | | | | | |
| HBB | G10 | gCTTGCCC | 258 | CGG | chr | 5226968 | 5226990 |
| | | CACAGGGC | | | 11 | | |
| | | AGTAA | | | | | |
| HBB | RO1 | GTGAACGT | 259 | tGG | chr | 5226945 | 5226967 |
| | | GGATGAAG | | | 11 | | |
| | | TTGG | | | | | |
| HBB | R03 | gACGTTCA | 260 | gGG | chr | 5226960 | 5226981 |
| | | CCTTGCCC | | | 11 | | |
| | | CACA | | | | | |
| HBB | R04 | gCACGTTC | 261 | aGG | chr | 5226959 | 5226980 |
| | | ACCTTGCC | | | 11 | | |
| | | ACCC | | | | | |
| VEGFA | sgRNA1 | GGGTGGGG | 262 | TGG | chr | 43769554 | 43769576 |
| | | GGAGTTTG | | | 6 | | |
| | | CTCC | | | | | |
| VEGFA | sgRNA2 | GACCCCCT | 263 | CGG | chr | 43770819 | 43770841 |
| | | CCACCCCG | | | 6 | | |
| | | CCTC | | | | | |
| VEGFA | sgRNA3 | GGTGAGTG | 264 | TGG | chr | 43769717 | 43769739 |
| | | AGTGTGTG | | | 6 | | |
| | | CGTG | | | | | |
| ZSCAN2 | sgRNA1 | GTGCGGCA | 265 | GGG | chr | 84621797 | 84621819 |
| | | AGAGCTTC | | | 15 | | |
| | | AGCC | | | | | |

TABLE 8
Off Target Loci.

| sgRNA | OT | Off-target | Chr | Start* | End* |
|---|---|---|---|---|---|
| CCR5 | R-30 | CCR2 | 3 | 46357652 | 46357674 |
| HBB | G10 | OT1 | 9 | 101833584 | 101833606 |
| HBB | R-01 | HBD | 11 | 5234357 | 5234379 |
| HBB | R-03 | HBD | 11 | 5234371 | 5234393 |
| HBB | R-04 | HBD | 11 | 5234370 | 5234392 |
| FANCF | sgRNA2 | OT1 | 22 | 36556948 | 36556970 |
| VEGFA | sgRNA1 | OT1 | 15 | 65345193 | 65345215 |
| VEGFA | sgRNA1 | OT4 | 12 | 131205637 | 131205659 |
| VEGFA | sgRNA1 | OT6 | 12 | 1878894 | 1878916 |
| VEGFA | sgRNA1 | OT11 | 1 | 98882089 | 98882111 |
| VEGFA | sgRNA2 | OT1 | 15 | 32993900 | 32993922 |
| VEGFA | sgRNA2 | OT2 | 11 | 31795929 | 31795951 |
| VEGFA | sgRNA2 | OT17 | 9 | 100837361 | 100837383 |
| VEGFA | sgRNA2 | OT19 | 2 | 241275175 | 241275197 |
| VEGFA | sgRNA3 | OT2 | 14 | 65102435 | 65102457 |
| VEGFA | sgRNA3 | OT4 | 22 | 37266777 | 37266799 |
| VEGFA | sgRNA3 | OT18 | 5 | 116098962 | 116098984 |
| ZSCAN2 | sgRNA1 | OT1 | 19 | 43914070 | 43914092 |
| ZSCAN2 | sgRNA1 | OT2 | 6 | 71610662 | 71610684 |

*indicates hg38 coordinates

TABLE 9
HDR Donors

| | Asso- | | SEQ | |
|---|---|---|---|---|
| | ciated | | ID | |
| HDR Donor | guide(s) | Sequence | NO: | Length |
| Symmetric | BFP | GAAGTCGTGCTGCT | | |
| BFP donor | sgRNA1/ | TCATGTGGTCGGGG | 266 | 100 |
| (sym) | sgRNA2 | TAGCGGCTGAAGCA | | |
| | | CTGCACGCCATAGG | | |
| | | TCAGGGTGGTCACG | | |
| | | AGGGTGGGCCAGGG | | |
| | | CACGGGCAGCTTGC | | |
| | | CG | | |
| Asymmetric | BFP | GCCACCTACGGCAA | | |
| BFP donor | sgRNA1/ | GCTGACCCTGAAGT | | |
| (asym) | sgRNA2 | TCATCTGCACCACC | 267 | 127 |
| | | GGCAAGCTGCCCGT | | |
| | | GCCCTGGCCCACCC | | |
| | | TCGTGACCACCCTG | | |
| | | ACCTATGGCGTGCA | | |
| | | GTGCTTCAGCCGCT | | |
| | | ACCCCGACCACATG | | |
| | | A | | |

While illustrative embodiments have been illustrated and described, it will be appreciated that various changes can be made therein without departing from the spirit and scope of the invention.

The embodiments of the invention in which an exclusive property or privilege is claimed are defined as follows:

1. A method of inhibiting off-target cleavage of a DNA molecule by a first guide RNA-endonuclease complex, wherein the first guide RNA-endonuclease complex comprises a first guide RNA comprising a nucleotide target recognition sequence complementary to a first target sequence, the method comprising:
contacting the DNA molecule with a second guide RNA-endonuclease complex, wherein the second guide RNA-endonuclease complex comprises a second guide RNA comprising a nucleotide target recognition sequence with 16 or fewer nucleotides and is complementary to a second target sequence in the DNA molecule, wherein the second target sequence is different from the first target sequence but the second target sequence is capable of cleavage at a measurable rate by the first guide RNA-endonuclease complex.

2. The method of claim 1, further comprising contacting the DNA molecule with the first guide RNA-endonuclease complex, and wherein second guide RNA-endonuclease complex is contacted to the DNA molecule prior to or simultaneously with the first guide RNA-endonuclease complex.

3. The method of claim 2, wherein the first guide RNA-endonuclease complex and the second guide RNA-endonuclease complex are contacted to the DNA molecule at a ratio of about 20:1 to about 1:20.

4. The method of claim 1, wherein the second target sequence differs from the first target sequence by 0-10 nucleotide mismatches.

5. The method of claim 1, wherein the first guide RNA-endonuclease complex comprises a first endonuclease and the second guide RNA-endonuclease complex comprises a second endonuclease, wherein the first endonuclease and the second endonuclease are clustered regularly interspersed short palindromic repeats (CRISPR)/CRISPR-associated (Cas) system proteins.

6. The method of claim 5, wherein the first endonuclease and the second endonuclease are independently selected from Cas12a, Cas9, eSpCas9, SpCas9-HF1, HypaCas9, xCas9, and SpCas9-NG.

7. The method of claim 1, wherein the nucleotide target recognition sequence of the second guide RNA-endonuclease complex comprises between 10 and 16 nucleotides inclusive that are complementary to the second target sequence.

8. The method of claim 1, wherein the method is multiplexed with one or more additional guide RNA-endonuclease complexes, wherein each of the one or more additional complexes comprises a different nucleotide target recognition sequence with 16 or fewer nucleotides and is complementary to one or more additional target sites in the DNA molecule or a plurality of DNA molecules in a same reaction environment, wherein the one or more additional target sequences are different from each other and from the first target sequence but the additional target sequences are capable of cleavage at measurable rates by the first guide RNA-endonuclease complex.

9. The method of claim 1, wherein the DNA molecule is in a cell, and wherein contacting the DNA molecule with the second guide RNA-endonuclease complex comprises contacting the cell with one or more exogenous nucleic acid molecules comprising a first sequence encoding the second guide RNA and a second sequence encoding the second endonuclease, wherein upon expression of the first sequence and the second sequence the second guide RNA and the second endonuclease form the second guide RNA-endonuclease complex in the cell.

10. The method of claim 1, wherein the DNA molecule is in a cell, and wherein contacting the DNA molecule with the second guide RNA-endonuclease complex comprises contacting the cell with a pre-assembled second guide RNA-endonuclease complex.

11. The method of claim 2, wherein the DNA molecule is in a cell, and wherein contacting the DNA molecule with the first guide RNA-endonuclease complex comprises contacting the cell with one or more exogenous nucleic acid molecules comprising a first sequence encoding the first guide RNA and a second sequence encoding a first endonuclease, wherein upon expression of the first sequence and the second sequence the first guide RNA and the first endonuclease form the first guide RNA-endonuclease complex in the cell.

12. The method of claim 2, wherein the DNA molecule is in a cell, and wherein contacting the DNA molecule with the first guide RNA-endonuclease complex comprises contacting the cell with a pre-assembled first guide RNA-endonuclease complex.

13. A method of inhibiting cleavage of a DNA molecule at a target site that has been previously modified from containing a first sequence to containing a second sequence by targeted cleavage by a first guide RNA-endonuclease complex and subsequent homology-directed repair (HDR), wherein the first guide RNA-endonuclease complex comprises a first guide RNA comprising a nucleotide target recognition sequence complementary to the first sequence, the method comprising:
contacting the DNA molecule with a second guide RNA-endonuclease complex, wherein the second guide RNA-endonuclease complex comprises a second guide RNA comprising a nucleotide target recognition sequence with 16 or fewer nucleotides and is complementary to at least a portion of the second sequence in the DNA molecule, wherein the second sequence is different from the first sequence but the second sequence is capable of cleavage at a measurable rate by the first guide RNA-endonuclease complex.

14. The method of claim 13, further comprising inducing targeted cleavage of the DNA molecule containing the first sequence by contacting the DNA molecule with the first guide RNA-endonuclease complex, thereby producing a cleaved DNA molecule, and contacting the cleaved DNA molecule with a repair polynucleotide that is substantially homologous to the target site but comprises the second sequence.

15. The method of claim 13, wherein the second sequence differs from the first sequence by 0-10 nucleotide mismatches.

16. The method of claim 13, wherein the first guide RNA-endonuclease complex comprises a first endonuclease and the second guide RNA-endonuclease complex comprises a second endonuclease, wherein the first endonuclease and the second endonuclease are clustered regularly interspersed short palindromic repeats (CRISPR)/CRISPR-associated (Cas) system proteins.

17. The method of claim 16, wherein the wherein the first endonuclease and second endonuclease are independently selected from Cas12a, Cas9, eSpCas9, SpCas9-HF1, HypaCas9, and xCas9, SpCas9-NG.

18. The method of claim 13, wherein the DNA molecule is in a cell, and wherein contacting the DNA molecule with the second guide RNA-endonuclease complex comprises contacting the cell with one or more exogenous DNA molecules comprising a first sequence encoding the second guide RNA and a second sequence encoding the second endonuclease, wherein upon expression of the first sequence and the second sequence the second guide RNA and the second endonuclease form the second guide RNA-endonuclease complex in the cell.

19. The method of claim 13, wherein the DNA molecule is in a cell, and wherein contacting the DNA molecule with the second guide RNA-endonuclease complex comprises contacting the cell with a pre-assembled second guide RNA-endonuclease complex.

20. A composition comprising a first guide RNA-endonuclease complex and a second guide RNA-endonuclease complex, wherein the guide RNA of the first guide RNA-endonuclease complex comprises a nucleotide target recognition sequence complementary to a first target sequence in a DNA molecule, wherein the guide RNA of the second guide RNA-endonuclease complex comprises a nucleotide target recognition sequence with 16 or fewer nucleotides and is complementary to a second target site in the DNA molecule or a distinct DNA molecule, wherein the second target sequence is different from the first target sequence but the second target sequence is capable of cleavage at a measurable rate by the first guide RNA-endonuclease complex.


SEQUENCE LISTING (267 sequences)

SEQ ID NO: 1 - DNA, 50 bp, Artificial sequence
  Synthetic
tcgtgaccaccctgacccatggcgtgcagtgcttcagccgctaccccgac

SEQ ID NO: 2 - DNA, 50 bp, Artificial sequence
  Synthetic
tcgtgaccaccctgacctatggcgtgcagtgcttcagccgctaccccgac

SEQ ID NO: 3 - DNA, 50 bp, Artificial sequence
  Synthetic
agcactggtgggactggataccgcacgtcacgaagtcggcgatggggctg

SEQ ID NO: 4 - DNA, 23 bp, Artificial sequence
  Synthetic
  misc_feature (3)..(3): n is a, c, g, or t
ggntaccgcacgtcacgaagtcg

SEQ ID NO: 5 - DNA, 18 bp, Artificial sequence
  Synthetic
  misc_feature (3)..(3): n is a, c, g, or t
ggnactggataccgcacg

SEQ ID NO: 6 - DNA, 17 bp, Artificial sequence
  Synthetic
  misc_feature (3)..(3): n is a, c, g, or t
ggnctggataccgcacg

SEQ ID NO: 7 - DNA, 17 bp, Artificial sequence
  Synthetic
  misc_feature (3)..(3): n is a, c, g, or t
ggngggactggataccg

SEQ ID NO: 8 - DNA, 19 bp, Artificial sequence
  Synthetic
gcagaagggattccaaggg

SEQ ID NO: 9 - DNA, 17 bp, Artificial sequence
  Synthetic
gaagggattccaagggg

SEQ ID NO: 10 - DNA, 23 bp, Artificial sequence
  Synthetic
gctgcagaagggattccatgagg

SEQ ID NO: 11 - DNA, 50 bp, Artificial sequence
  Synthetic
aaaagcgatccaggtgctgcagaagggattccatgaggtgcgcgaaggcc

SEQ ID NO: 12 - DNA, 50 bp, Artificial sequence
  Synthetic
cacctcatccactatgctgcagaagggattccaaggggaatacgaagtca

SEQ ID NO: 13 - DNA, 50 bp, Artificial sequence
  Synthetic
gtggagtaggtgatacgacgtcttccctaaggttccccttatgcttcagt

SEQ ID NO: 14 - DNA, 19 bp, Artificial sequence
  Synthetic
ggttccccttatgcttcag

SEQ ID NO: 15 - DNA, 18 bp, Artificial sequence
  Synthetic
ggagtaggtgatacgacg

SEQ ID NO: 16 - DNA, 53 bp, Artificial sequence
  Synthetic
tcgtcggcagcgtcagatgtgtataagagacagtgaccctgaagttcatctgc

SEQ ID NO: 17 - DNA, 54 bp, Artificial sequence
  Synthetic
gtctcgtgggctcggagatgtgtataagagacagggtcttgtagttgccgtcgt

SEQ ID NO: 18 - DNA, 53 bp, Artificial sequence
  Synthetic
tcgtcggcagcgtcagatgtgtataagagacaggctcttcagccttttgcagt

SEQ ID NO: 19 - DNA, 54 bp, Artificial sequence
  Synthetic
gtctcgtgggctcggagatgtgtataagagacagcagggtggaacaagatggat

SEQ ID NO: 20 - DNA, 54 bp, Artificial sequence
  Synthetic
tcgtcggcagcgtcagatgtgtataagagacagaagcacttcagctttttgcag

SEQ ID NO: 21 - DNA, 54 bp, Artificial sequence
  Synthetic
gtctcgtgggctcggagatgtgtataagagacagatgattacggtgctccctgt

SEQ ID NO: 22 - DNA, 53 bp, Artificial sequence
  Synthetic
tcgtcggcagcgtcagatgtgtataagagacagcccaggtgctgacgtaggta

SEQ ID NO: 23 - DNA, 54 bp, Artificial sequence
  Synthetic
gtctcgtgggctcggagatgtgtataagagacagagcattgcagagaggcgtat

SEQ ID NO: 24 - DNA, 53 bp, Artificial sequence
  Synthetic
tcgtcggcagcgtcagatgtgtataagagacagcctccagccctactgactga

SEQ ID NO: 25 - DNA, 54 bp, Artificial sequence
  Synthetic
gtctcgtgggctcggagatgtgtataagagacagcccactctctcctgttctgg

SEQ ID NO: 26 - DNA, 53 bp, Artificial sequence
  Synthetic
tcgtcggcagcgtcagatgtgtataagagacaggcaacctcaaacagacacca

SEQ ID NO: 27 - DNA, 54 bp, Artificial sequence
  Synthetic
gtctcgtgggctcggagatgtgtataagagacaggtctccacatgcccagtttc

SEQ ID NO: 28 - DNA, 53 bp, Artificial sequence
  Synthetic
tcgtcggcagcgtcagatgtgtataagagacagaggggaagatcccagagaac

SEQ ID NO: 29 - DNA, 54 bp, Artificial sequence
  Synthetic
gtctcgtgggctcggagatgtgtataagagacagtttccaggctatgcttccat

SEQ ID NO: 30 - DNA, 53 bp, Artificial sequence
  Synthetic
tcgtcggcagcgtcagatgtgtataagagacaggtttccatttgcctccttga

SEQ ID NO: 31 - DNA, 54 bp, Artificial sequence
  Synthetic
gtctcgtgggctcggagatgtgtataagagacaggcaacctcaaacagacacca

SEQ ID NO: 32 - DNA, 56 bp, Artificial sequence
  Synthetic
tcgtcggcagcgtcagatgtgtataagagacagggctctctgtacatgaagcaact

SEQ ID NO: 33 - DNA, 53 bp, Artificial sequence
  Synthetic
gtctcgtgggctcggagatgtgtataagagacagcctagtgactgccgtctgc

SEQ ID NO: 34 - DNA, 53 bp, Artificial sequence
  Synthetic
tcgtcggcagcgtcagatgtgtataagagacagacctggccatcatccttcta

SEQ ID NO: 35 - DNA, 54 bp, Artificial sequence
  Synthetic
gtctcgtgggctcggagatgtgtataagagacagcagcagacccactgagtcaa

SEQ ID NO: 36 - DNA, 53 bp, Artificial sequence
  Synthetic
tcgtcggcagcgtcagatgtgtataagagacagcctgcaggtgtctccttttc

SEQ ID NO: 37 - DNA, 53 bp, Artificial sequence
  Synthetic
gtctcgtgggctcggagatgtgtataagagacaggcacctgcaatgtcagagg

SEQ ID NO: 38 - DNA, 53 bp, Artificial sequence
  Synthetic
tcgtcggcagcgtcagatgtgtataagagacagctcagcacctgcacttcttg

SEQ ID NO: 39 - DNA, 54 bp, Artificial sequence
  Synthetic
gtctcgtgggctcggagatgtgtataagagacagcagatgtggccctgagagag

SEQ ID NO: 40 - DNA, 54 bp, Artificial sequence
  Synthetic
tcgtcggcagcgtcagatgtgtataagagacaggagttgtcctgcagctgtacc

SEQ ID NO: 41 - DNA, 54 bp, Artificial sequence
  Synthetic
gtctcgtgggctcggagatgtgtataagagacagcaaggcatctctgccttcat

SEQ ID NO: 42 - DNA, 51 bp, Artificial sequence
  Synthetic
tcgtcggcagcgtcagatgtgtataagagacagccccagctaccacctcct

SEQ ID NO: 43 - DNA, 54 bp, Artificial sequence
  Synthetic
gtctcgtgggctcggagatgtgtataagagacaggaacagcccagaagttggac

SEQ ID NO: 44 - DNA, 53 bp, Artificial sequence
  Synthetic
tcgtcggcagcgtcagatgtgtataagagacaggcagtactccctgctgtcct

SEQ ID NO: 45 - DNA, 55 bp, Artificial sequence
  Synthetic
gtctcgtgggctcggagatgtgtataagagacagggtttcccaatttcatcttca

SEQ ID NO: 46 - DNA, 53 bp, Artificial sequence
  Synthetic
tcgtcggcagcgtcagatgtgtataagagacaggcagcctattgtctcctggt

SEQ ID NO: 47 - DNA, 54 bp, Artificial sequence
  Synthetic
gtctcgtgggctcggagatgtgtataagagacagcttgcctgtaaggccacagt

SEQ ID NO: 48 - DNA, 53 bp, Artificial sequence
  Synthetic
tcgtcggcagcgtcagatgtgtataagagacagctcccatgaggggtttgagt

SEQ ID NO: 49 - DNA, 54 bp, Artificial sequence
  Synthetic
gtctcgtgggctcggagatgtgtataagagacagttgcacaagaacctgctgtc

SEQ ID NO: 50 - DNA, 53 bp, Artificial sequence
  Synthetic
tcgtcggcagcgtcagatgtgtataagagacagcatttgtccaggaaccccta

SEQ ID NO: 51 - DNA, 54 bp, Artificial sequence
  Synthetic
gtctcgtgggctcggagatgtgtataagagacaggctttgggcttttagcctct

SEQ ID NO: 52 - DNA, 52 bp, Artificial sequence
  Synthetic
tcgtcggcagcgtcagatgtgtataagagacaggcagcgtcttcgagagtga

SEQ ID NO: 53 - DNA, 54 bp, Artificial sequence; identical to SEQ ID NO: 43
  Synthetic

SEQ ID NO: 54 - DNA, 53 bp, Artificial sequence
  Synthetic
tcgtcggcagcgtcagatgtgtataagagacagagggacccctctgacagact

SEQ ID NO: 55 - DNA, 54 bp, Artificial sequence
  Synthetic
gtctcgtgggctcggagatgtgtataagagacagcagccctcagacttcacatt

SEQ ID NO: 56 - DNA, 51 bp, Artificial sequence
  Synthetic
tcgtcggcagcgtcagatgtgtataagagacaggaggggaaggggtgaagg

SEQ ID NO: 57 - DNA, 54 bp, Artificial sequence
  Synthetic
gtctcgtgggctcggagatgtgtataagagacaggcagtgaggaggtggttctt

SEQ ID NO: 58 - DNA, 51 bp, Artificial sequence
  Synthetic
tcgtcggcagcgtcagatgtgtataagagacagggcccatttctcctttga

SEQ ID NO: 59 - DNA, 54 bp, Artificial sequence
  Synthetic
gtctcgtgggctcggagatgtgtataagagacagttaggagagctggcttggaa

SEQ ID NO: 60 - DNA, 55 bp, Artificial sequence
  Synthetic
tcgtcggcagcgtcagatgtgtataagagacaggggaatctaatgtatggcatgg

SEQ ID NO: 61 - DNA, 54 bp, Artificial sequence
  Synthetic
gtctcgtgggctcggagatgtgtataagagacagcgtattcagggtgtgcaatg

SEQ ID NO: 62 - DNA, 51 bp, Artificial sequence
  Synthetic
tcgtcggcagcgtcagatgtgtataagagacaggctcccagctcgtagtgc

SEQ ID NO: 63 - DNA, 54 bp, Artificial sequence
  Synthetic
gtctcgtgggctcggagatgtgtataagagacaggttccagctaaagcctttcc

SEQ ID NO: 64 - DNA, 53 bp, Artificial sequence
  Synthetic
tcgtcggcagcgtcagatgtgtataagagacagcccacatgtaccacatttgt

SEQ ID NO: 65 - DNA, 55 bp, Artificial sequence
  Synthetic
gtctcgtgggctcggagatgtgtataagagacagccgtatcagtgtgatgcatgt

SEQ ID NO: 66 - DNA, 53 bp, Artificial sequence
  Synthetic
tcgtcggcagcgtcagatgtgtataagagacagtgtggcacaaagtggaagag

SEQ ID NO: 67 - DNA, 54 bp, Artificial sequence
  Synthetic
gtctcgtgggctcggagatgtgtataagagacagcagagcctttattgcccatc

SEQ ID NO: 68 - DNA, 51 bp, Artificial sequence
  Synthetic
aatgatacggcgaccaccgagatctacacgctaactatcgtcggcagcgtc

SEQ ID NO: 69 - DNA, 51 bp, Artificial sequence
  Synthetic
aatgatacggcgaccaccgagatctacaccacggggctcgtcggcagcgtc

SEQ ID NO: 70 - DNA, 51 bp, Artificial sequence
  Synthetic
aatgatacggcgaccaccgagatctacacaataccagtcgtcggcagcgtc

SEQ ID NO: 71 - DNA, 51 bp, Artificial sequence
  Synthetic
aatgatacggcgaccaccgagatctacacagtctcgctcgtcggcagcgtc

SEQ ID NO: 72 - DNA, 51 bp, Artificial sequence
  Synthetic
aatgatacggcgaccaccgagatctacactcgtcagctcgtcggcagcgtc

SEQ ID NO: 73 - DNA, 51 bp, Artificial sequence
  Synthetic
aatgatacggcgaccaccgagatctacacgagcttaatcgtcggcagcgtc

SEQ ID NO: 74 - DNA, 51 bp, Artificial sequence
  Synthetic
aatgatacggcgaccaccgagatctacacgccattgttcgtcggcagcgtc

SEQ ID NO: 75 - DNA, 51 bp, Artificial sequence
  Synthetic
aatgatacggcgaccaccgagatctacacatgccagatcgtcggcagcgtc

SEQ ID NO: 76 - DNA, 51 bp, Artificial sequence
  Synthetic
aatgatacggcgaccaccgagatctacacctgtgctttcgtcggcagcgtc

SEQ ID NO: 77 - DNA, 51 bp, Artificial sequence
  Synthetic
aatgatacggcgaccaccgagatctacacggagtcaatcgtcggcagcgtc

SEQ ID NO: 78 - DNA, 51 bp, Artificial sequence
  Synthetic
aatgatacggcgaccaccgagatctacacagaacaactcgtcggcagcgtc

SEQ ID NO: 79 - DNA, 51 bp, Artificial sequence
  Synthetic
aatgatacggcgaccaccgagatctacaccacaccattcgtcggcagcgtc

SEQ ID NO: 80 - DNA, 51 bp, Artificial sequence
  Synthetic
aatgatacggcgaccaccgagatctacacagcgattttcgtcggcagcgtc

SEQ ID NO: 81 - DNA, 51 bp, Artificial sequence
  Synthetic
aatgatacggcgaccaccgagatctacacgacgcggctcgtcggcagcgtc

SEQ ID NO: 82 - DNA, 51 bp, Artificial sequence
  Synthetic
aatgatacggcgaccaccgagatctacactggcctgttcgtcggcagcgtc

SEQ ID NO: 83 - DNA, 51 bp, Artificial sequence
  Synthetic
aatgatacggcgaccaccgagatctacacgagcgacatcgtcggcagcgtc

SEQ ID NO: 84 - DNA, 51 bp, Artificial sequence
  Synthetic
aatgatacggcgaccaccgagatctacacaaagcttttcgtcggcagcgtc

SEQ ID NO: 85 - DNA, 51 bp, Artificial sequence
  Synthetic
aatgatacggcgaccaccgagatctacacaatgggaatcgtcggcagcgtc

SEQ ID NO: 86 - DNA, 51 bp, Artificial sequence
  Synthetic
aatgatacggcgaccaccgagatctacactcccgtaatcgtcggcagcgtc

SEQ ID NO: 87 - DNA, 51 bp, Artificial sequence
  Synthetic
aatgatacggcgaccaccgagatctacactcttcaaatcgtcggcagcgtc

SEQ ID NO: 88 - DNA, 51 bp, Artificial sequence
  Synthetic
aatgatacggcgaccaccgagatctacacattctcaatcgtcggcagcgtc

SEQ ID NO: 89 - DNA, 51 bp, Artificial sequence
  Synthetic
aatgatacggcgaccaccgagatctacaccctgcttttcgtcggcagcgtc

SEQ ID NO: 90 - DNA, 51 bp, Artificial sequence
  Synthetic
aatgatacggcgaccaccgagatctacacactaagcgtcgtcggcagcgtc

SEQ ID NO: 91 - DNA, 51 bp, Artificial sequence
  Synthetic
aatgatacggcgaccaccgagatctacacctgggtcctcgtcggcagcgtc

SEQ ID NO: 92 - DNA, 51 bp, Artificial sequence
  Synthetic
aatgatacggcgaccaccgagatctacactactccagtcgtcggcagcgtc

SEQ ID NO: 93 - DNA, 48 bp, Artificial sequence
  Synthetic
caagcagaagacggcatacgagattacgaagtcgtctcgtgggctcgg

SEQ ID NO: 94 - DNA, 48 bp, Artificial sequence
  Synthetic
caagcagaagacggcatacgagatgacgagattgtctcgtgggctcgg

SEQ ID NO: 95 - DNA, 48 bp, Artificial sequence
  Synthetic
caagcagaagacggcatacgagataccgtaagagtctcgtgggctcgg

SEQ ID NO: 96 - DNA, 48 bp, Artificial sequence
  Synthetic
caagcagaagacggcatacgagattagtggcaagtctcgtgggctcgg

SEQ ID NO: 97 - DNA, 48 bp, Artificial sequence
  Synthetic
caagcagaagacggcatacgagatcattaacgcgtctcgtgggctcgg

SEQ ID NO: 98 - DNA, 48 bp, Artificial sequence
  Synthetic
caagcagaagacggcatacgagattcgttgaaggtctcgtgggctcgg

SEQ ID NO: 99 - DNA, 48 bp, Artificial sequence
  Synthetic
caagcagaagacggcatacgagattagtacgctgtctcgtgggctcgg

SEQ ID NO: 100 - DNA, 48 bp, Artificial sequence
  Synthetic
caagcagaagacggcatacgagatctcagatcagtctcgtgggctcgg

SEQ ID NO: 101 - DNA, 48 bp, Artificial sequence
  Synthetic
caagcagaagacggcatacgagatttcaccgtagtctcgtgggctcgg

SEQ ID NO: 102 - DNA, 48 bp, Artificial sequence
  Synthetic
caagcagaagacggcatacgagatgtcatgcatgtctcgtgggctcgg

SEQ ID NO: 103 - DNA, 48 bp, Artificial sequence
  Synthetic
caagcagaagacggcatacgagataggacagttgtctcgtgggctcgg

SEQ ID NO: 104 - DNA, 48 bp, Artificial sequence
  Synthetic
caagcagaagacggcatacgagatatggtgtctgtctcgtgggctcgg

SEQ ID NO: 105 - DNA, 48 bp, Artificial sequence
  Synthetic
caagcagaagacggcatacgagatggatgttctgtctcgtgggctcgg

SEQ ID NO: 106 - DNA, 48 bp, Artificial sequence
  Synthetic
caagcagaagacggcatacgagatcttatccaggtctcgtgggctcgg

SEQ ID NO: 107 - DNA, 48 bp, Artificial sequence
  Synthetic
caagcagaagacggcatacgagatgtaagtcacgtctcgtgggctcgg

SEQ ID NO: 108 - DNA, 48 bp, Artificial sequence
  Synthetic
caagcagaagacggcatacgagatttcagtgaggtctcgtgggctcgg

SEQ ID NO: 109 - DNA, 48 bp, Artificial sequence
  Synthetic
caagcagaagacggcatacgagatctcgtaatggtctcgtgggctcgg

SEQ ID NO: 110 - DNA, 48 bp, Artificial sequence
  Synthetic
caagcagaagacggcatacgagatcatgtctcagtctcgtgggctcgg

SEQ ID NO: 111 - DNA, 48 bp, Artificial sequence
  Synthetic
caagcagaagacggcatacgagataatcgtggagtctcgtgggctcgg

SEQ ID NO: 112 - DNA, 48 bp, Artificial sequence
  Synthetic
caagcagaagacggcatacgagatgtatcagtcgtctcgtgggctcgg

SEQ ID NO: 113 - DNA, 48 bp, Artificial sequence
  Synthetic
caagcagaagacggcatacgagatagcagatgtgtctcgtgggctcgg

SEQ ID NO: 114 - DNA, 48 bp, Artificial sequence
  Synthetic
caagcagaagacggcatacgagattcctaacgtgtctcgtgggctcgg

SEQ ID NO: 115 - DNA, 48 bp, Artificial sequence
  Synthetic
caagcagaagacggcatacgagataacagtccagtctcgtgggctcgg

SEQ ID NO: 116 - DNA, 48 bp, Artificial sequence
  Synthetic
caagcagaagacggcatacgagatccttgagaagtctcgtgggctcgg

SEQ ID NO: 117 - DNA, 48 bp, Artificial sequence
  Synthetic
caagcagaagacggcatacgagatttaagcctggtctcgtgggctcgg

SEQ ID NO: 118 - DNA, 48 bp, Artificial sequence
  Synthetic
caagcagaagacggcatacgagatttagaccacgtctcgtgggctcgg

SEQ ID NO: 119 - DNA, 48 bp, Artificial sequence
  Synthetic
caagcagaagacggcatacgagattgtctagtggtctcgtgggctcgg

SEQ ID NO: 120 - DNA, 48 bp, Artificial sequence
  Synthetic
caagcagaagacggcatacgagattagatcgaggtctcgtgggctcgg

SEQ ID NO: 121 - DNA, 48 bp, Artificial sequence
  Synthetic
caagcagaagacggcatacgagattgaatgccagtctcgtgggctcgg

SEQ ID NO: 122 - DNA, 48 bp, Artificial sequence
  Synthetic
caagcagaagacggcatacgagatgtgcaatgtgtctcgtgggctcgg

SEQ ID NO: 123 - DNA, 48 bp, Artificial sequence
  Synthetic
caagcagaagacggcatacgagatagtggcatagtctcgtgggctcgg

SEQ ID NO: 124 - DNA, 48 bp, Artificial sequence
  Synthetic
caagcagaagacggcatacgagatatgatcggtgtctcgtgggctcgg

SEQ ID NO: 125 - DNA, 48 bp, Artificial sequence
  Synthetic
caagcagaagacggcatacgagatagtctacctgtctcgtgggctcgg

SEQ ID NO: 126 - DNA, 48 bp, Artificial sequence
  Synthetic
caagcagaagacggcatacgagatgatcaactggtctcgtgggctcgg

SEQ ID NO: 127 - DNA, 48 bp, Artificial sequence
  Synthetic
caagcagaagacggcatacgagatatcggtagtgtctcgtgggctcgg

SEQ ID NO: 128 - DNA, 48 bp, Artificial sequence
  Synthetic
caagcagaagacggcatacgagatcgtatgatggtctcgtgggctcgg

SEQ ID NO: 129 - DNA, 48 bp, Artificial sequence
  Synthetic
caagcagaagacggcatacgagatttactgacggtctcgtgggctcgg

SEQ ID NO: 130 - DNA, 48 bp, Artificial sequence
  Synthetic
caagcagaagacggcatacgagatctgtcgtaagtctcgtgggctcgg

SEQ ID NO: 131 - DNA, 48 bp, Artificial sequence
  Synthetic
caagcagaagacggcatacgagattcaactggtgtctcgtgggctcgg

SEQ ID NO: 132 - DNA, 48 bp, Artificial sequence
  Synthetic
caagcagaagacggcatacgagatatcgatctcgtctcgtgggctcgg

SEQ ID NO: 133 - DNA, 48 bp, Artificial sequence
  Synthetic
caagcagaagacggcatacgagatgcaactatggtctcgtgggctcgg

SEQ ID NO: 134 - DNA, 48 bp, Artificial sequence
  Synthetic
caagcagaagacggcatacgagatgatgacttcgtctcgtgggctcgg

SEQ ID NO: 135 - DNA, 48 bp, Artificial sequence
  Synthetic
caagcagaagacggcatacgagatgacgttacagtctcgtgggctcgg

SEQ ID NO: 136 - DNA, 48 bp, Artificial sequence
  Synthetic
caagcagaagacggcatacgagatcatctgctagtctcgtgggctcgg

SEQ ID NO: 137 - DNA, 48 bp, Artificial sequence
  Synthetic
caagcagaagacggcatacgagatattagtcgggtctcgtgggctcgg

SEQ ID NO: 138 - DNA, 48 bp, Artificial sequence
  Synthetic
caagcagaagacggcatacgagattagcgtactgtctcgtgggctcgg

SEQ ID NO: 139 - DNA, 48 bp, Artificial sequence
  Synthetic
caagcagaagacggcatacgagatccaagcaatgtctcgtgggctcgg

SEQ ID NO: 140 - DNA, 48 bp, Artificial sequence
  Synthetic
caagcagaagacggcatacgagatccgtaattggtctcgtgggctcgg

SEQ ID NO: 141 - DNA, 463 bp, Homo sapiens
ggttctccagcaggcgcagagagagcaggacgtcacagtgaccgagggcctggaagttcgctaatcccggaactggaccccgcccaaagccgccctcttgcctccactggttgtgcagccgccgctccagagccgtgcgaatggggccatgccgaccaaagcgccgatggatgtggcgcaggtagcgcgcccactgcaaggcccggcgcacggtggcggggtcccaggtgctgacgtaggtagtgcttgagaccgccagaagctcggaaaagcgatccaggtgctgcagaagggattccatgaggtgcgcgaaggccctacttccgctttcaccttggagacggcgactctctgcgtactgattggaacatccgcgaaatgatacgcctctctgcaatgctattggtcgaaatgcatgtcaatctcccagcgtctttatccgtgttccttgactctgggcaac

SEQ ID NO: 142 - DNA, 329 bp, Homo sapiens
cctgtctcccactctctcctgttctggctcccttgttttttctccctcctctctcttccaccgagttaccagcctctgtctcacctcatccactatgctgcagaagggattccaaggggaatacgaagtcagtcatatgaaacccaggcacctctgtcagtcagtagggctggaggtggagacagaaatggggccccagatgggatctctgaggcagccctttgagatgagtcccacaagatcaagaacatccctcccaccccattcattccaggcccgggatgaactatcacgatcctgaaacagttcaaatctcagcacctcacggg

SEQ ID NO: 143 - DNA, 125 bp, Artificial sequence
  Synthetic
ggatcctaatacgactcactatagctgcagaagggattccatggttttagagctagaaatagcaagttaaaataaggctagtccgttatcaacttgaaaaagtggcaccgagtcggtgctttttt

SEQ ID NO: 144 - DNA, 119 bp, Artificial sequence
  Synthetic
ggatcctaatacgactcactatagaagggattccaaggttttagagctagaaatagcaagttaaaataaggctagtccgttatcaacttgaaaaagtggcaccgagtcggtgctttttt

SEQ ID NO: 145 - DNA, 19 bp, Artificial sequence
  Synthetic
ggttctccagcaggcgcag

SEQ ID NO: 146 - DNA, 23 bp, Artificial sequence
  Synthetic
gttgcccagagtcaaggaacacg

SEQ ID NO: 147 - DNA, 21 bp, Artificial sequence
  Synthetic
cctgtctcccactctctcctg

SEQ ID NO: 148 - DNA, 21 bp, Artificial sequence
  Synthetic
cccgtgaggtgctgagatttg

SEQ ID NO: 149 - DNA, 15 bp, Artificial sequence
  Synthetic
gctggtgttcatctt

SEQ ID NO: 150 - DNA, 16 bp, Artificial sequence
  Synthetic
gcaccagcgagtagag

SEQ ID NO: 151 - DNA, 16 bp, Artificial sequence
  Synthetic
gcagcgagtagagcgg

SEQ ID NO: 152 - DNA, 15 bp, Artificial sequence
  Synthetic
gcggaggcaggagtt

SEQ ID NO: 153 - DNA, 16 bp, Artificial sequence
  Synthetic
gacgtgaagcaaattg

SEQ ID NO: 154 - DNA, 15 bp, Artificial sequence
  Synthetic
gcgtgaagcaaattg

SEQ ID NO: 155 - DNA, 16 bp, Artificial sequence
  Synthetic
gctccgctctactcgc

SEQ ID NO: 156 - DNA, 16 bp, Artificial sequence
  Synthetic
gccttactgccctgtg

SEQ ID NO: 157 - DNA, 16 bp, Artificial sequence
  Synthetic
gcccttactgccctgt

SEQ ID NO: 158 - DNA, 15 bp, Artificial sequence
  Synthetic
gcccttactgccctg

SEQ ID NO: 159 - DNA, 16 bp, Artificial sequence
  Synthetic
gccccacagggcagta

SEQ ID NO: 160 - DNA, 15 bp, Artificial sequence
  Synthetic
gaacgtggatgcagt

SEQ ID NO: 161 - DNA, 14 bp, Artificial sequence
  Synthetic
gtggatgcagttgg

SEQ ID NO: 162 - DNA, 15 bp, Artificial sequence
  Synthetic
gctgtcaatgccctg

SEQ ID NO: 163 - DNA, 16 bp, Artificial sequence
  Synthetic
gcccacagggcagtaa

SEQ ID NO: 164 - DNA, 15 bp, Artificial sequence
  Synthetic
gccgttactgccctg

SEQ ID NO: 165 - DNA, 16 bp, Artificial sequence
  Synthetic
gtcactttgccccaca

SEQ ID NO: 166 - DNA, 14 bp, Artificial sequence
  Synthetic
gaagggattccaag

SEQ ID NO: 167 - DNA, 16 bp, Artificial sequence
  Synthetic
gacttcgtattcccct

SEQ ID NO: 168 - DNA, 15 bp, Artificial sequence
  Synthetic
gcagcatagtggatg

SEQ ID NO: 169 - DNA, 15 bp, Artificial sequence
  Synthetic
gcagaagggattcca

SEQ ID NO: 170 - DNA, 16 bp, Artificial sequence
  Synthetic
gcagaagggattccaa

SEQ ID NO: 171 - DNA, 16 bp, Artificial sequence
  Synthetic
ggatttgtgggatgga

SEQ ID NO: 172 - DNA, 16 bp, Artificial sequence
  Synthetic
ggagggagtttgctcc

SEQ ID NO: 173 - DNA, 15 bp, Artificial sequence
  Synthetic
gagggagtttgctcc

SEQ ID NO: 174 - DNA, 15 bp, Artificial sequence
  Synthetic
gggagtttgctcctg

SEQ ID NO: 175 - DNA, 16 bp, Artificial sequence
  Synthetic
gggtggagtttgctcc

SEQ ID NO: 176 - DNA, 15 bp, Artificial sequence
  Synthetic
ggtggagtttgctcc

SEQ ID NO: 177 - DNA, 16 bp, Artificial sequence
  Synthetic
gttatgatagggaggg

SEQ ID NO: 178 - DNA, 16 bp, Artificial sequence
  Synthetic
gcccattatgataggg

SEQ ID NO: 179 - DNA, 15 bp, Artificial sequence
  Synthetic
gtggagtttgctcct

SEQ ID NO: 180 - DNA, 14 bp, Artificial sequence
  Synthetic
ggagtttgctcctg

SEQ ID NO: 181 - DNA, 15 bp, Artificial sequence
  Synthetic
gtttgctcctgggga

SEQ ID NO: 182 - DNA, 15 bp, Artificial sequence
  Synthetic
ggcccttccatcccc

SEQ ID NO: 183 - DNA, 15 bp, Artificial sequence
  Synthetic
gagggagtttgctcc

SEQ ID NO: 184 - DNA, 16 bp, Artificial sequence
  Synthetic
gtcccatcacggggga

SEQ ID NO: 185 - DNA, 16 bp, Artificial sequence
  Synthetic
gaggctcccatcacgg

SEQ ID NO: 186 - DNA, 14 bp, Artificial sequence
  Synthetic
ggctcccatcacgg

SEQ ID NO: 187 - DNA, 14 bp, Artificial sequence
  Synthetic
gggagtttgctcct

SEQ ID NO: 188 - DNA, 15 bp, Artificial sequence
  Synthetic
gggagtttgctcctg

SEQ ID NO: 189 - DNA, 15 bp, Artificial sequence
  Synthetic
gatcacaggttcccc

SEQ ID NO: 190 - DNA, 15 bp, Artificial sequence
  Synthetic
ggggaagtttgctcc

SEQ ID NO: 191 - DNA, 16 bp, Artificial sequence
  Synthetic
gctcctggcattcagt

SEQ ID NO: 192 - DNA, 15 bp, Artificial sequence
  Synthetic
gctcctggcattcag

SEQ ID NO: 193 - DNA, 16 bp, Artificial sequence
  Synthetic
gtcacaactcggggag

SEQ ID NO: 194 - DNA, 15 bp, Artificial sequence
  Synthetic
gtcacaactcgggga

SEQ ID NO: 195 - DNA, 14 bp, Artificial sequence
  Synthetic
gtcacaactcgggg

SEQ ID NO: 196 - DNA, 14 bp, Artificial sequence
  Synthetic
gctgtcacaactcg

SEQ ID NO: 197 - DNA, 16 bp, Artificial sequence
  Synthetic
gtacccactgaatgcc

SEQ ID NO: 198 - DNA, 16 bp, Artificial sequence
  Synthetic
gcccccaccccgcccc

SEQ ID NO: 199 - DNA, 16 bp, Artificial sequence
  Synthetic
ggtggggggggtcttt

SEQ ID NO: 200 - DNA, 15 bp, Artificial sequence
  Synthetic
gtggggggggtcttt

SEQ ID NO: 201 - DNA, 14 bp, Artificial sequence
  Synthetic
ggctgctgttgcag

SEQ ID NO: 202 - DNA, 16 bp, Artificial sequence
  Synthetic
gggggcggggtggggg

SEQ ID NO: 203 - DNA, 16 bp, Artificial sequence
  Synthetic
gccaaggcgctcctag

SEQ ID NO: 204 - DNA, 16 bp, Artificial sequence
  Synthetic
gtctggccaagttttg

SEQ ID NO: 205 - DNA, 15 bp, Artificial sequence
  Synthetic
ggtggaggggcccct

SEQ ID NO: 206 - DNA, 16 bp, Artificial sequence
  Synthetic
gccagaggcggggtgg

SEQ ID NO: 207 - DNA, 14 bp, Artificial sequence
  Synthetic
ggccagaggcgggg

SEQ ID NO: 208 - DNA, 16 bp, Artificial sequence
  Synthetic
gacttggccagaggcg

SEQ ID NO: 209 - DNA, 16 bp, Artificial sequence
  Synthetic
gcccccaccccgcctc

SEQ ID NO: 210 - DNA, 16 bp, Artificial sequence
  Synthetic
gttggacgtcctgagg

SEQ ID NO: 211 - DNA, 14 bp, Artificial sequence
  Synthetic
gtccaacagggttg

SEQ ID NO: 212 - DNA, 16 bp, Artificial sequence
  Synthetic
gcccccaccccgcctc

SEQ ID NO: 213 - DNA, 16 bp, Artificial sequence
  Synthetic
ggtttattctttcctg

SEQ ID NO: 214 - DNA, 16 bp, Artificial sequence
  Synthetic
gtgaggcggggtgggg

SEQ ID NO: 215 - DNA, 14 bp, Artificial sequence
  Synthetic
gaggcggggtgggg

SEQ ID NO: 216 - DNA, 16 bp, Artificial sequence
  Synthetic
gctgaggcggggtggg

SEQ ID NO: 217 - DNA, 16 bp, Artificial sequence
  Synthetic
gttcctgaggcggggt

SEQ ID NO: 218 - DNA, 16 bp, Artificial sequence
  Synthetic
gaataaacctcatacc

SEQ ID NO: 219 - DNA, 15 bp, Artificial sequence
  Synthetic
gtgagtgtgtgtgtg

SEQ ID NO: 220 - DNA, 16 bp, Artificial sequence
  Synthetic
gagtgagtgtgtgtgt

SEQ ID NO: 221 - DNA, 16 bp, Artificial sequence
  Synthetic
gtgtgtgggggggact

SEQ ID NO: 222 - DNA, 15 bp, Artificial sequence
  Synthetic
gtgagtgtatgcgtg

SEQ ID NO: 223 - DNA, 15 bp, Artificial sequence
  Synthetic
gcgtgtggctttagc

SEQ ID NO: 224 - DNA, 14 bp, Artificial sequence
  Synthetic
gcgtgtggctttag

SEQ ID NO: 225 - DNA, 16 bp, Artificial sequence
  Synthetic
ggctttagcgggaagc

SEQ ID NO: 226 - DNA, 15 bp, Artificial sequence
  Synthetic
gctttagcgggaagc

SEQ ID NO: 227 - DNA, 16 bp, Artificial sequence
  Synthetic
gccaccttttatgtgt

SEQ ID NO: 228 - DNA, 16 bp, Artificial sequence
  Synthetic
gaccaccttttatgtg

SEQ ID NO: 229 - DNA, 16 bp, Artificial sequence
  Synthetic
gtcacccacacataaa

SEQ ID NO: 230 - DNA, 16 bp, Artificial sequence
  Synthetic
gcccacacataaaagg

SEQ ID NO: 231 - DNA, 16 bp, Artificial sequence
  Synthetic
ggcaagggcttcagcc

SEQ ID NO: 232 - DNA, 15 bp, Artificial sequence
  Synthetic
gcaagggcttcagcc

SEQ ID NO: 233 - DNA, 14 bp, Artificial sequence
  Synthetic
ggcctcaaatcttc

SEQ ID NO: 234 - DNA, 16 bp, Artificial sequence
  Synthetic
gtgaggagtgtggcaa

SEQ ID NO: 235 - DNA, 14 bp, Artificial sequence
  Synthetic
gaagatttgaggcc

SEQ ID NO: 236 - DNA, 16 bp, Artificial sequence
  Synthetic
gggaagagcttcagca

SEQ ID NO: 237 - DNA, 15 bp, Artificial sequence
  Synthetic
ggaagagcttcagca

SEQ ID NO: 238 - DNA, 16 bp, Artificial sequence
  Synthetic
gaagctcttccctcac

SEQ ID NO: 239 - DNA, 14 bp, Artificial sequence
  Synthetic
gctcttccctcaca

SEQ ID NO: 240 - DNA, 16 bp, Artificial sequence
  Synthetic
gagctgttccctgtga

SEQ ID NO: 241 - DNA, 15 bp, Artificial sequence
  Synthetic
gcacgccataggtca

SEQ ID NO: 242 - DNA, 14 bp, Artificial sequence
  Synthetic
gcacgccataggtc

SEQ ID NO: 243 - DNA, 14 bp, Artificial sequence
  Synthetic
gccataggtcaggg

SEQ ID NO: 244 - DNA, 20 bp, Homo sapiens
gtagagcggaggcaggaggc

SEQ ID NO: 245 - DNA, 20 bp, Homo sapiens
gctgcagaagggattccatg

SEQ ID NO: 246 - DNA, 21 bp, Homo sapiens
gcttgccccacagggcagtaa

SEQ ID NO: 247 - DNA, 20 bp, Homo sapiens
gtgaacgtggatgaagttgg

SEQ ID NO: 248 - DNA, 20 bp, Homo sapiens
gacgttcaccttgccccaca

SEQ ID NO: 249 - DNA, 20 bp, Homo sapiens
gcacgttcaccttgccccac

SEQ ID NO: 250 - DNA, 20 bp, Homo sapiens
gggtggggggagtttgctcc

SEQ ID NO: 251 - DNA, 20 bp, Homo sapiens
gaccccctccaccccgcctc

SEQ ID NO: 252 - DNA, 20 bp, Homo sapiens
ggtgagtgagtgtgtgcgtg

SEQ ID NO: 253 - DNA, 17 bp, Homo sapiens
gagtgagtgtgtgcgtg

SEQ ID NO: 254 - DNA, 20 bp, Homo sapiens
gtgcggcaagagcttcagcc

SEQ ID NO: 255 - DNA, 20 bp, Artificial sequence
  Synthetic
gctgaagcactgcacgccat

SEQ ID NO: 256 - DNA, 20 bp, Homo sapiens
gtagagcggaggcaggaggc

SEQ ID NO: 257 - DNA, 20 bp, Homo sapiens
gctgcagaagggattccatg

SEQ ID NO: 258 - DNA, 21 bp, Homo sapiens
gcttgccccacagggcagtaa

SEQ ID NO: 259 - DNA, 20 bp, Homo sapiens
gtgaacgtggatgaagttgg

SEQ ID NO: 260 - DNA, 20 bp, Homo sapiens
gacgttcaccttgccccaca

SEQ ID NO: 261 - DNA, 20 bp, Homo sapiens
gcacgttcaccttgccccac

SEQ ID NO: 262 - DNA, 20 bp, Homo sapiens
gggtggggggagtttgctcc

SEQ ID NO: 263 - DNA, 20 bp, Homo sapiens
gaccccctccaccccgcctc

SEQ ID NO: 264 - DNA, 20 bp, Homo sapiens
ggtgagtgagtgtgtgcgtg

SEQ ID NO: 265 - DNA, 20 bp, Homo sapiens
gtgcggcaagagcttcagcc

SEQ ID NO: 266 - DNA, 100 bp, Artificial sequence
  Synthetic
gaagtcgtgctgcttcatgtggtcggggtagcggctgaagcactgcacgccataggtcagggtggtcacgagggtgggccagggcacgggcagcttgccg

SEQ ID NO: 267 - DNA, 127 bp, Artificial sequence
  Synthetic
gccacctacggcaagctgaccctgaagttcatctgcaccaccggcaagctgcccgtgccctggcccaccctcgtgaccaccctgacctatggcgtgcagtgcttcagccgctaccccgaccacatga